(19) United States
(12) Patent Application Publication
(10) Pub. No.: US 2026/0098092 A1
(43) Pub. Date: Apr. 9, 2026

(54) MONOCLONAL ANTIBODIES AGAINST HUMAN PROGRAMMED CELL DEATH PROTEIN 1 (PD-1)

(71) Applicant: Chulalongkorn University, Bangkok (TH)

(72) Inventors: Trairak Pisitkun, Bangkok (TH); Chatikorn Boonkrai, Bangkok (TH); Tossapon Wongtangprasert, Bangkok (TH); Tanapati Phakham, Bangkok (TH); Thittaya Audomsun; Chadaporn Attakitbancha, Bangkok (TH); Pijitra Saelao, Bangkok (TH)

(21) Appl. No.: 19/114,741

(22) PCT Filed: Oct. 4, 2023
(86) PCT No.: PCT/US2023/075954

§ 371 Date: Mar. 24, 2025

Related U.S. Application Data
(60) Provisional application No. 63/378,472, filed on Oct. 5, 2022.

(51) Int. Cl. C07K 16/28; A61K 39/00; A61P 35/00

(52) U.S. Cl. CPC C07K 16/2818; A61P 35/00; A61K 2039/505; C07K 2317/24; C07K 2317/52; C07K 2317/565; C07K 2317/76; C07K 2317/92

(57) ABSTRACT

The present disclosure relates to antibodies and anti-gen-binding fragments that bind specifically to the human programmed cell death protein 1 (PD-1) and methods for treating cancer or other diseases with said antibodies and antigen-binding fragments.

### CROSS-REFERENCE TO RELATED APPLICATIONS

The present application claims the benefit of U.S. Provisional Patent Application No. 63/378,472, filed Oct. 5, 2022, which is incorporated herein by reference in its entirety.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “FMCU-002_001 WO_SeqList”. The XML file is 311,500 bytes, created on Oct. 4, 2023, and is being submitted electronically via USPTO Patent Center.

### TECHNICAL FIELD

The present disclosure relates to antibodies and antigen-binding fragments that bind specifically to the human programmed cell death protein 1 (PD-1) and methods for treating cancer or other diseases with said antibodies and antigen-binding fragments.

### BACKGROUND

Cancer has become a leading cause of death globally in the past decades, and its incidence still increases every year. Recently, cancer immunotherapy has become a promising treatment strategy by harnessing a patient's immune system to battle cancer (Marin-Acevedo et al., J Hematol Oncol. 2018; 11(1):8; Waldman et al., Nat Rev Immunol. 2020; 20(11):651-68).

Immune checkpoint inhibitors (ICIs) are drugs targeting immune checkpoint molecules, currently the most successful cancer immunotherapy agents (Pardoll et al., Nat Rev Cancer. 2012; 12(4):252-64; Hargadon et al., Int Immunopharmacol. 2018:62:29-39).

Among approved ICIs are monoclonal antibodies against programmed cell death protein 1 (PD-1). PD-1 is an immunosuppressive co-stimulatory signal receptor that is expressed primarily on activated T cells and B cells. It controls inappropriate and extreme immune responses such as autoimmune and excessive infectious immune responses and suppresses antigen receptor activation by PD-ligand 1 (PD-L1) (Chamoto et al., Int J Oncol. 25:790-800 (2020)).

In spite of their initial success, a substantial number of patients remains unresponsive to PD-1 targeted therapy, and side effects associated with the autoimmune response limit the clinical potential of these drugs. Thus, there remains a need for new inhibitors of PD-1 and/or PD-L1.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### SUMMARY

In one aspect, provided herein is an anti-PD-1 antibody or antigen-binding fragment thereof comprising
(a) (i) a heavy chain variable region (VH) comprising a complementarity-determining region (CDR)1 comprising the sequence set forth in SEQ ID NO: 81, a CDR2 comprising the sequence set forth in SEQ ID NO: 82, and a CDR3 comprising the sequence set forth in SEQ ID NO: 83; and (ii) comprising a light chain variable region (VL) comprising a CDR1 comprising the sequence set forth in SEQ ID NO: 85, a CDR2 comprising the sequence set forth in SEQ ID NO: 86, and a CDR3 comprising the sequence set forth in SEQ ID NO: 87;
(b) (i) a VH comprising a CDR1 comprising the sequence set forth in SEQ ID NO: 89, a CDR2 comprising the sequence set forth in SEQ ID NO: 90, and a CDR3 comprising the sequence set forth in SEQ ID NO: 91; and (ii) comprising a VL comprising a CDR1 comprising the sequence set forth in SEQ ID NO: 93, a CDR2 comprising the sequence set forth in SEQ ID NO: 94, and a CDR3 comprising the sequence set forth in SEQ ID NO: 95;
(c) (i) a VH comprising a CDR1 comprising the sequence set forth in SEQ ID NO: 97, a CDR2 comprising the sequence set forth in SEQ ID NO: 98, and a CDR3 comprising the sequence set forth in SEQ ID NO: 99; and (ii) comprising a VL comprising a CDR1 comprising the sequence set forth in SEQ ID NO: 101, a CDR2 comprising the sequence set forth in SEQ ID NO: 102, and a CDR3 comprising the sequence set forth in SEQ ID NO: 103;
(d) (i) a VH comprising a CDR1 comprising the sequence set forth in SEQ ID NO: 105, a CDR2 comprising the sequence set forth in SEQ ID NO: 106, and a CDR3 comprising the sequence set forth in SEQ ID NO: 107; and (ii) comprising a VL comprising a CDR1 comprising the sequence set forth in SEQ ID NO: 109, a CDR2 comprising the sequence set forth in SEQ ID NO: 110, and a CDR3 comprising the sequence set forth in SEQ ID NO: 111;
(e) (i) a VH comprising a CDR1 comprising the sequence set forth in SEQ ID NO: 113, a CDR2 comprising the sequence set forth in SEQ ID NO: 114, and a CDR3 comprising the sequence set forth in SEQ ID NO: 115; and (ii) comprising a VL comprising a CDR1 comprising the sequence set forth in SEQ ID NO: 117, a CDR2 comprising the sequence set forth in SEQ ID NO: 118, and a CDR3 comprising the sequence set forth in SEQ ID NO: 119;
(f) (i) a VH comprising a CDR1 comprising the sequence set forth in SEQ ID NO: 121, a CDR2 comprising the sequence set forth in SEQ ID NO: 122, and a CDR3 comprising the sequence set forth in SEQ ID NO: 123; and (ii) comprising a VL comprising a CDR1 comprising the sequence set forth in SEQ ID NO: 125, a CDR2 comprising the sequence set forth in SEQ ID NO: 126, and a CDR3 comprising the sequence set forth in SEQ ID NO: 127; or
(g) (i) a VH comprising a CDR1 comprising the sequence set forth in SEQ ID NO: 129, a CDR2 comprising the sequence set forth in SEQ ID NO: 130, and a CDR3 comprising the sequence set forth in SEQ ID NO: 131; and (ii) comprising a VL comprising a CDR1 comprising the sequence set forth in SEQ ID NO: 133, a CDR2 comprising the sequence set forth in SEQ ID NO: 134, and a CDR3 comprising the sequence set forth in SEQ ID NO: 135.

In some embodiments, the anti-PD-1 antibody or antigen binding fragment thereof comprises a VH comprising a sequence that is at least 80%, at least 85%, at least 90%, at least 95%, at least 96%, at least 97%, at least 98%, or at least 99% identical to the sequence set forth in any one of SEQ ID NOs: 80, 88, 96, 104, 112, 120, 128, 136, 137, 138, 139, 140, 144, 145, 146, 147, 148, 149, 153, 154, 155, and 156. In some embodiments, the anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in any one of SEQ ID NOs: 84, 92, 100, 108, 116, 124, 132, 141, 142, 143, 150, 151, 152, 157, and 158. In some embodiments, the anti-PD-1 antibody or antigen-binding fragment thereof comprises a VH comprising the sequence set forth in any one of SEQ ID NOs: 80, 88, 96, 104, 112, 120, 128, 136, 137, 138, 139, 140, 144, 145, 146, 147, 148, 149, 153, 154, 155, and 156. In some embodiments, the anti-PD-1 antibody or antigen-binding fragment thereof comprises a VL comprising the sequence set forth in any one of SEQ ID NOs: 84, 92, 100, 108, 116, 124, 132, 141, 142, 143, 150, 151, 152, 157, and 158.

In some embodiments, the anti-PD-1 antibody or antigen-binding fragment thereof comprises
(a) a VH comprising the sequence set forth in SEQ ID NO: 136 and a VL comprising the sequence set forth in SEQ ID NO: 143;
(b) a VH comprising the sequence set forth in SEQ ID NO: 137 and a VL comprising the sequence set forth in SEQ ID NO: 143;
(c) a VH comprising the sequence set forth in SEQ ID NO: 138 and a VL comprising the sequence set forth in SEQ ID NO: 143;
(d) a VH comprising the sequence set forth in SEQ ID NO: 139 and a VL comprising the sequence set forth in SEQ ID NO: 143;
(e) a VH comprising the sequence set forth in SEQ ID NO: 145 and a VL comprising the sequence set forth in SEQ ID NO: 150;
(f) a VH comprising the sequence set forth in SEQ ID NO: 145 and a VL comprising the sequence set forth in SEQ ID NO: 151;
(g) a VH comprising the sequence set forth in SEQ ID NO: 145 and a VL comprising the sequence set forth in SEQ ID NO: 152;
(h) a VH comprising the sequence set forth in SEQ ID NO: 146 and a VL comprising the sequence set forth in SEQ ID NO: 150;
(g) a VH comprising the sequence set forth in SEQ ID NO: 146 and a VL comprising the sequence set forth in SEQ ID NO: 151;
(h) a VH comprising the sequence set forth in SEQ ID NO: 146 and a VL comprising the sequence set forth in SEQ ID NO: 152.
(i) a VH comprising the sequence set forth in SEQ ID NO: 153 and a VL comprising the sequence set forth in SEQ ID NO: 157;
(j) a VH comprising the sequence set forth in SEQ ID NO: 154 and a VL comprising the sequence set forth in SEQ ID NO: 158.
(k) a VH comprising the sequence set forth in SEQ ID NO: 155 and a VL comprising the sequence set forth in SEQ ID NO: 158; or
(l) a VH comprising the sequence set forth in SEQ ID NO: 156 and a VL comprising the sequence set forth in SEQ ID NO: 158.

In some embodiments, the antibody is a monoclonal antibody. In some embodiments, the antibody or antigen-binding fragment thereof is a humanized antibody or antigen-binding fragment thereof. In some embodiments, the antibody or antigen-binding fragment thereof is an IgG1 antibody or antigen-binding fragment thereof. In some embodiments, the antigen-binding fragment is a Fab fragments, Fab′ fragments, F(ab′)2 fragments, scFv, dsFv, ds-scFv, or a nanobody. In some embodiments, the antibody has a L234AL235A mutation in Fc region.

In some embodiments, the antibody or antigen-binding fragment thereof binds to PD-1 with a K_{D} of less than 0.5 nM, less than 0.3 nM, or less than 0.1 nM, less than 0.05 nM, less than 0.02 nM. In some embodiments, the antibody or antigen-binding fragment inhibits the interaction between PD-1 and PD-L1 with an IC₅₀ of less than about 500 ng/mL, less than about 400 ng/mL, or less than about 300 ng/mL.

In some embodiments, the antibody or antigen-binding fragment activates T cells. In some embodiments, the antibody or antigen-binding fragment induces IL-2 secretion in T cells. In some embodiments, the antibody or antigen-binding fragment thereof induces IFN-γ secretion in T cells.

In another aspect, provided herein is a pharmaceutical composition comprising an anti-PD-1 antibody or antigen-binding fragment thereof provided herein and a pharmaceutically acceptable carrier.

In another aspect, provided herein is a method of treating cancer in a subject, comprising administering to the subject an anti-PD-1 antibody or antigen-binding fragment thereof or a pharmaceutical composition provided herein. In some embodiments, the cancer is colon cancer, breast cancer, lung cancer, ovarian cancer, prostate cancer, cervical cancer, brain cancer, skin cancer, liver cancer, pancreatic cancer or stomach cancer. In some embodiments, the cancer is a leukemia or a lymphoma.

In another aspect, provided herein is a method of treating an infectious disease in a subject, comprising administering to the subject an anti-PD-1 antibody or antigen-binding fragment thereof or a pharmaceutical composition provided herein. In some embodiments, the infectious disease is tuberculosis, malaria, an HIV infection, or a HBV infection.

In another aspect, provided herein is a method of treating primary immunodeficiency in a subject, comprising administering to the subject an anti-PD-1 antibody or antigen-binding fragment thereof or a pharmaceutical composition provided herein.

In another aspect, provided herein is an anti-PD-1 antibody or antigen-binding fragment thereof for the treatment of cancer. In some embodiments, the cancer is colon cancer, breast cancer, lung cancer, ovarian cancer, prostate cancer, cervical cancer, brain cancer, skin cancer, liver cancer, pancreatic cancer or stomach cancer. In some embodiments, the cancer is a leukemia or a lymphoma.

In another aspect, provided herein is an anti-PD-1 antibody or antigen-binding fragment thereof or a pharmaceutical composition comprising an ani-PD-1 antibody or antigen-binding fragment thereof for the treatment of an infectious disease. In some embodiments, the infectious disease is tuberculosis, malaria, an HIV infection, or a HBV infection.

In another aspect, provided herein is an anti-PD-1 antibody or antigen-binding fragment thereof or a pharmaceutical composition comprising an anti-PD-1 antibody or antigen-binding fragment thereof for the treatment of primary immunodeficiency.

In another aspect, provided herein is the use of an anti-PD-1 antibody or antigen-binding fragment thereof provided herein in the manufacture of a medicament for the treatment of cancer. In some embodiments, the cancer is colon cancer, breast cancer, lung cancer, ovarian cancer, prostate cancer, cervical cancer, brain cancer, skin cancer, liver cancer, pancreatic cancer or stomach cancer. In some embodiments, the cancer is a leukemia or a lymphoma.

In another aspect, provided herein is the use of an anti-PD-1 antibody or antigen-binding fragment thereof provided herein in the manufacture of a medicament for the treatment of an infectious disease. In some embodiments, the infectious disease is tuberculosis, malaria, an HIV infection, or a HBV infection.

In another aspect, provided herein is the use of an anti-PD-1 antibody or antigen-binding fragment thereof provided herein in the manufacture of a medicament for the treatment of primary immunodeficiency.

In another aspect, provided herein is a method of inhibiting the interaction between PD-1 and PD-L1 in a cell, comprising contacting the cell with an antibody or antigen-binding fragment thereof of provided herein. In another aspect, provided herein is a method of inhibiting the interaction between PD-1 and PD-L2 in a cell, comprising contacting the cell with an antibody or antigen-binding fragment thereof provided herein. In another aspect, provided herein is a method of stimulating IL-2 secretion in a cell, comprising contacting the cell with an antibody or antigen-binding fragment thereof provided herein. In another aspect, provided herein is a method of stimulating IFN-γ secretion in a cell, comprising contacting the cell with an antibody or antigen-binding fragment thereof provided herein. In some embodiments, the cell is a human cell. In some embodiments, the cell is contacted in vitro. In some embodiments, the cell is contacted in vivo.

### BRIEF DESCRIPTION OF THE DRAWINGS

FIGS. 1A and 1B show the (A) PD-1 binding profiles and (B) PD-1/PD-L1 neutralization profiles of mouse anti-PD-1 antibodies.

FIGS. 2A and 2B show the (A) PD-1 binding profiles and (B) PD-1/PD-L1 neutralization profiles of chimeric anti-PD-1 antibodies.

FIGS. 3A and 3B show the PD-1/PD-L1 neutralizing profile of humanized anti-PD-1 antibodies of clone CUSB0125 and clone CUSB0136 evaluated by PD-1/PD-L1 blockade bioassay.

FIGS. 4A-4D show IL-2 and IFN-γ secretion from human T cells evaluated by MLR assay. FIGS. 4A and 4B are a result of clone CUSB0125. FIGS. 4C and 4D are a result of clone CUSB0136.

FIGS. 5A and 5B show in vivo anti-tumor efficacy of humanized anti-PD-1 antibodies at dose 10 mg/kg.

FIGS. 6A and 6B show in vivo anti-tumor efficacy of zCUSB0136.8-LALA anti-PD-1 antibody at dose 5 and 10 mg/kg.

FIG. 7 shows sequence alignments of some of the anti-PD-1 antibodies described herein.

FIGS. 8A and 8B show sequence logo analysis of the VH and VL of some of the anti-PD-1 antibodies described herein. Each logo consists of stacks of symbols, one stack for each position in the sequence. The overall height of the stack indicates the sequence conservation at that position, while the height of symbols within the stack indicates the relative frequency of each amino or nucleic acid at that position

FIGS. 9A and 9B show in vivo anti-tumor efficacy of affinity-matured zCUSB0136.8-2 anti-PD-1 antibody at dose 2.5 and 10 mg/kg, respectively.

FIG. 10 shows the PD-1/PD-L2 neutralizing profile of affinity-matured zCUSB0136.8-2 anti-PD-1 antibody evaluated by flow cytometry.

### DETAILED DESCRIPTION

### Definitions

It is to be understood that, unless otherwise stated, any description of a method of treatment or prevention includes use of the peptides to provide such treatment or prevention as is described herein. It is to be further understood, unless otherwise stated, any description of a method of treatment or prevention includes use of the peptides to prepare a medicament to treat or prevent such condition. The treatment or prevention includes treatment or prevention of human or non-human animals including rodents and other disease models.

It is to be understood that, unless otherwise stated, any description of a method of treatment includes use of the peptides to provide such treatment as is described herein. It is to be further understood, unless otherwise stated, any description of a method of treatment includes use of the peptides to prepare a medicament to treat such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In some embodiments, the mammal is a human. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a peptide of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a peptide of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al, Molecular Cloning, A Laboratory Manual (3^{rd} edition), Cold Spring Harbor Press, Cold Spring Harbor. New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

### Antibodies

Provided herein are antibodies and antigen-binding fragments thereof that bind to PD-1. also provided herein are pharmaceutical compositions comprising the anti-PD-1 antibodies or antigen binding fragments thereof provided herein and methods of using the same.

In one aspect, provided herein are antibodies and antigen-binding fragments thereof that bind to PD-1, also referred to as “anti-PD-1 antibodies.” The term “antibody” may refer to an immunoglobulin tetramer structure comprising two wo identical pairs of polypeptide chains, each pair comprising one light chain and one heavy chain, or to other antibody structures, such as single-domain antibodies or multispecific antibodies. Thus, the term “antibody” may also refer to a single-chain antibody, a bispecific antibody, a trispecific antibody, and other multispecific antibodies such as diabodies, triabodies and tetrabodies. Each light chain has a variable domain at one end (VL) and a constant domain at its other end. Each heavy chain has a variable domain (VH) at the N-terminus, and has three constant domains (CH) for α and γ chains and four CH domains for μ and ε isotypes.

The term “antigen-binding fragment” includes, but is not limited to, Fab fragments, Fab′ fragments, F(ab′)2 fragments, Fd, Fv, scFv, dsFv, ds-scFv, dimers, minibodies, diabodies, and multimers thereof, domain antibodies, dual-specific antibodies, minibodies, scap (sterol regulatory binding protein cleavage activating protein), chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies.

The variable region of an antibody or antigen-binding fragment thereof comprises the antigen-binding site, which is responsible for antigen binding specificity and affinity. In particular, six hypervariable regions, three heavy chain complementarity-determining regions (CDRs) and three light chain CDRs, form the antigen binding site. The more conserved portions of variable domains are called the framework regions (FR). Thus, the light and heavy chain variable domains comprise from N- to C-terminus the domains FR1, CDR1, FR2, CDR2, FR-3, CDR3 and FR4.

In some embodiments, an anti-PD-1 antibody or antigen-binding fragment thereof comprises one or more CDR (e.g., three VH CDRs and three VL CDRSs) each comprising a CDR sequence set forth in

Table 1. In some embodiments, an anti-PD-1 antibody comprises one or more CDR (e.g., three VH CDRs and three VL CDRs) each consisting of a CDR sequence set forth in Table 1. It will be apparent to a person of skill in the art that the variable and light chains of different antibodies may be freely combined. Thus, for example, an anti-PD-1 antibody may comprise the VH of the antibody with the description “CUSB0103” in Table 1 and the VL of the antibody with the description “CUSB123” in Table 1.

Several methods of defining the CDRs of an antibody are known in the at, including, for example, Kabat, Chothia, AbM, Contact, and IMGT. The CDRs in

Table 1 are defined using the Kabat definition. However, a person of skill will be able to determine the CDRs of the CH and VL sequences set forth in
Table 1 using another definition known in the art.

TABLE 1
Amino acid sequences of anti-PD-1 antibodies

| SEQ ID | | |
|---|---|---|
| NO | Description | Sequence |
| 80 | CUSB0103- | DVKLVESGGGLVKPGGSLKLSCAASGFTFSSYTMSWVRQTPE |
| | VH | KRLEWVAAISGGGSYTYYADSVKGRFTISRDNAKNTLSLQMS |
| | | SLKSEDTAMYFCTSPYDFAMDYWGQGTSVTVSS |
| 81 | CUSB0103- | SYTMS |
| | VH-CDR1 | |
| 82 | CUSB0103- | AISGGGSYTYYADSVKG |
| | VH-CDR2 | |
| 83 | CUSB0103- | PYDFAMDY |
| | VH-CDR3 | |
| 84 | CUSB0103- | DIQMTQTTSSLSASLGDRVTISCRASQDITNYLNWYQQKPDGS |
| | VL | VKLLIYYTSRLHSGVPSRFRGSGSGTDYSLTISNLEQEDIATYFC |
| | | QQGNTLPWTFGGGTKLEIK |
| 85 | CUSB0103- | RASQDITNYLN |
| | VL-CDR1 | |
| 86 | CUSB0103- | YTSRLHS |
| | VL-CDR2 | |
| 87 | CUSB0103- | QQGNTLPWT |
| | VL-CDR3 | |
| 88 | CUSB0123- | QVQLQQSGPELVRPGESVKISCKGSGYTFTDYVMHWVKQSHA |
| | VH | KSLEWIGVITIYNDITNYNQKFKGKATMTIDKSSSTAYMELAR |
| | | LTSEDSAIYYCAREGYDSYWYFDVWGAGTTVTVSS |
| 89 | CUSB0123- | DYVMH |
| | VH-CDR1 | |
| 90 | CUSB0123- | VITIYNDITNYNQKFKG |
| | VH-CDR2 | |
| 91 | CUSB0123- | EGYDSYWYFDV |
| | VH-CDR3 | |
| 92 | CUSB0123- | DVLMTQTPLSLPVSLGDQASISCRSSQTIVHSNGNTYLEWYLQ |
| | VL | KPGQSPKLLIYKVSNRFSGVPDRFSGSGSGTDFTLKISRVEAED |
| | | LGVYYCFQGSHLPYTFGGGTKLEIK |
| 93 | CUSB0123- | RSSQTIVHSNGNTYLE |
| | VL-CDR1 | |
| 94 | CUSB0123- | KVSNRFS |
| | VL-CDR2 | |
| 95 | CUSB0123- | FQGSHLPYT |
| | VL-CDR3 | |
| 96 | CUSB0124- | QIQLVQSGPELKKPGETVKISCKASGYIFTNYGVTWVKQTPGK |
| | VH | GLKWMTWINTNTGEPTSAEEFKGRFDFSSETSATTAYLQINNL |
| | | KNEDTATYFCVRIRENAMDYWGQGTSVTVSS |
| 97 | CUSB0124- | NYGVT |
| | VH-CDR1 | |
| 98 | CUSB0124- | WINTNTGEPTSAEEFKG |
| | VH-CDR2 | |
| 99 | CUSB0124- | IRENAMDY |
| | VH-CDR3 | |
| 100 | CUSB0124- | DIQMTQSPASQSASLGESVTITCLASQTIGTWLAWYQQKPGKS |
| | VL | PQLLIYAATRLAVGVPSRFSGSGSGTKFSFKISRLQAEDFVSYY |
| | | CQQLYSTPFTFGSGTKLEIK |
| 101 | CUSB0124- | LASQTIGTWLA |
| | VL-CDR1 | |
| 102 | CUSB0124- | AATRLAV |
| | VL-CDR2 | |
| 103 | CUSB0124- | QQLYSTPFT |
| | VL-CDR3 | |
| 104 | CUSB0125- | DVQLRESGPGLVKPSQSLSLTCTVTGYSITSDYAWNWIRQFPG |
| | VH | NKLEWMGYIYYSGTTSYNPSLKSRISITRDTSKNQFFLQLNSVT |
| | | TEDTATYYCARNYGSAFYYFDYWGQGTTLTVSS |
| 105 | CUSB0125- | SDYAWN |
| | VH-CDR1 | |
| 106 | CUSB0125- | YIYYSGTTSYNPSLKS |
| | VH-CDR2 | |
| 107 | CUSB0125- | NYGSAFYYFDY |
| | VH-CDR3 | |
| 108 | CUSB0125- | DILMTQSPSSMSVSLGDTVTITCHASQGISSNIGWLQQKPGKSF |
| | VL | KGLIYRGTNLEDGVPSRFSGSGSGADYSLTISSLESEDFADYYC |
| | | VQYAQFPPTFGGGTKLEIK |
| 109 | CUSB0125- | HASQGISSNIG |
| | VL-CDR1 | |
| 110 | CUSB0125- | RGTNLED |
| | VL-CDR2 | |
| 111 | CUSB0125- | VQYAQFPPT |
| | VL-CDR3 | |
| 112 | CUSB0126- | VKLVESGGDLVKPGGSLKLSCAASGFPFSSYLMSWVRQTPEK |
| | VH | RLEWVAAISGGGRDTYYPDNVKGRFTISRDNARNILYLQMSSL |
| | | RSEDTAVYYCATRGSYAMDYWGQGTSVTVSS |
| 113 | CUSB0126- | SYLMS |
| | VH-CDR1 | |
| 114 | CUSB0126- | AISGGGRDTYYPDNVKG |
| | VH-CDR2 | |
| 115 | CUSB0126- | RGSYAMDY |
| | VH-CDR3 | |
| 116 | CUSB0126- | DIQMTQSPASQSASLGESVTITCLASQTIGTWLAWYQQKPGKS |
| | VL | PQILIYAATNLADGVPSRFSGSGSGTKFSFKISSLQAEDFVSYYC |
| | | QQLYSIPWTFGGGTKLEIK |
| 117 | CUSB0126- | LASQTIGTWLA |
| | VL-CDR1 | |
| 118 | CUSB0126- | AATNLAD |
| | VL-CDR2 | |
| 119 | CUSB0126- | QQLYSIPWT |
| | VL-CDR3 | |
| 120 | CUSB0129- | VKLVESGGGLVKPGGSLKLSCAASGFPFSSYLMSWVRQTPEK |
| | VH | RLEWVAAISGGGRDTYYPDSVKGRFTISRDNARNILYLQMSSL |
| | | RSEDTALYYCARRGSYAMDYWGQGTSVTVSS |
| 121 | CUSB0129- | SYLMS |
| | VH-CDR1 | |
| 122 | CUSB0129- | AISGGGRDTYYPDSVKG |
| | VH-CDR2 | |
| 123 | CUSB0129- | RGSYAMDY |
| | VH-CDR3 | |
| 124 | CUSB0129- | DIQMTQSPASQSASLGESVTITCLASQTIGTWLAWYQQKPGKS |
| | VL | PQILIYDATNLADGVPSRFSGSGSGTKFSFKISSLQAEDFVSYYC |
| | | QQLYSIPWTFGGGTKLEIK |
| 125 | CUSB0129- | LASQTIGTWLA |
| | VL-CDR1 | |
| 126 | CUSB0129- | DATNLAD |
| | VL-CDR2 | |
| 127 | CUSB0129- | QQLYSIPWT |
| | VL-CDR3 | |
| 128 | CUSB0136- | QVQLKESGPGLVAPSQSLSITCTVSGFSLTTYDIIWIRQSPGKGL |
| | VH | EWLGVIWTGGDTNYNSAFMSRLSISKDNSKSQIFLKMNSLQTD |
| | | DTAIYYCVRDWAYWGQGTLVTVST |
| 129 | CUSB0136- | TYDII |
| | VH-CDR1 | |
| 130 | CUSB0136- | VIWTGGDTNYNSAFMS |
| | VH-CDR2 | |
| 131 | CUSB0136- | DWAY |
| | VH-CDR3 | |
| 132 | CUSB0136- | NIVLTQSPASLAVSLGQRATISCRASESVDNYGNSFIQWYQQKP |
| | VL | GQPPKLLIYFASNLQSGVPARFSGSGSRTDFTLTIDPVEADDAA |
| | | TYYCHQNNEDPFTFGSGTKLEIK |
| 133 | CUSB0136- | RASESVDNYGNSFIQ |
| | VL-CDR1 | |
| 134 | CUSB0136- | FASNLQS |
| | VL-CDR2 | |
| 135 | CUSB0136- | HQNNEDPFT |
| | VL-CDR3 | |
| 136 | CUSB0125- | QVQLQESGPGLVKPSQTLSLTCTVSGYSITSDYAWNWIRQPPG |
| | BMVH.1 | KGLEWIGYIYYSGTTSYNPSLKSRVTISVDTSKNQFSLKLSSVT |
| | | AADTAVYYCARNYGSAFYYFDYWGQGTTLTVSS |
| 159 | CUSB0125- | SDYAWN |
| | BMVH.1- | |
| | CDR1 | |
| 160 | CUSB0125- | YIYYSGTTSYNPSLKS |
| | BMVH.1- | |
| | CDR2 | |
| 161 | CUSB0125- | NYGSAFYYFDY |
| | BMVH.1- | |
| | CDR3 | |
| 137 | CUSB0125- | QVQLQESGPGLVKPSQTLSLTCTVSGYSITSDYAWNWIRQPPG |
| | BMVH.2 | KGLEWMGYIYYSGTTSYNPSLKSRITISRDTSKNQFSLKLSSVT |
| | | AADTATYYCARNYGSAFYYFDYWGQGTTLTVSS |
| 162 | CUSB0125- | SDYAWN |
| | BMVH.2- | |
| | CDR1 | |
| 163 | CUSB0125- | YIYYSGTTSYNPSLKS |
| | BMVH.2- | |
| | CDR2 | |
| 164 | CUSB0125- | NYGSAFYYFDY |
| | BMVH.2- | |
| | CDR3 | |
| 138 | CUSB0125- | QVQLQESGPGLVKPSQTLSLTCTVSGYSITSDYAWNWIRQPPG |
| | BMVH.3 | KGLEWMGYIYYSGTTSYNPSLKSRISITRDTSKNQFSLKLSSVT |
| | | AADTAVYYCARNYGSAFYYFDYWGQGTTLTVSS |
| 165 | CUSB0125- | SDYAWN |
| | BMVH.3- | |
| | CDR1 | |
| 166 | CUSB0125- | YIYYSGTTSYNPSLKS |
| | BMVH.3- | |
| | CDR2 | |
| 167 | CUSB0125- | NYGSAFYYFDY |
| | BMVH.3- | |
| | CDR3 | |
| 139 | CUSB0125- | QVQLQESGPGLVKPSQTLSLTCTVSGYSITSDYAWNWIRQFPG |
| | BMVH.4 | KKLEWMGYIYYSGTTSYNPSLKSRISITRDTSKNQFSLKLSSVT |
| | | AADTAVYYCARNYGSAFYYFDYWGQGTTLTVSS |
| 168 | CUSB0125- | SDYAWN |
| | BMVH.4- | |
| | CDR1 | |
| 169 | CUSB0125- | YIYYSGTTSYNPSLKS |
| | BMVH.4- | |
| | CDR2 | |
| 170 | CUSB0125- | NYGSAFYYFDY |
| | BMVH.4- | |
| | CDR3 | |
| 140 | CUSB0125- | QVQLQESGPGLVKPSQTLSLTCTVSGYSITSDYAWNWIRQFPG |
| | BMVH.5 | KKLEWMGYIYYSGTTSYNPSLKSRISITRDTSKNQFSLQLSSVT |
| | | AADTATYYCARNYGSAFYYFDYWGQGTTLTVSS |
| 171 | CUSB0125- | SDYAWN |
| | BMVH.5- | |
| | CDR1 | |
| 172 | CUSB0125- | YIYYSGTTSYNPSLKS |
| | BMVH.5- | |
| | CDR2 | |
| 173 | CUSB0125- | NYGSAFYYFDY |
| | BMVH.5- | |
| | CDR3 | |
| 141 | CUSB0125- | DIQMTQSPSSVSASVGDRVTITCHASQGISSNIGWYQQKPGKAP |
| | BMVL.1 | KLLIYRGTNLEDGVPSRFSGSGSGTDFTLTISSLQPEDFATYYC |
| | | VQYAQFPPTFGGGTKLEIK |
| 174 | CUSB0125- | HASQGISSNIG |
| | BMVL.1- | |
| | CDR1 | |
| 175 | CUSB0125- | RGTNLED |
| | BMVL.1- | |
| | CDR2 | |
| 176 | CUSB0125- | VQYAQFPPT |
| | BMVL.1- | |
| | CDR3 | |
| 142 | CUSB0125- | DIQMTQSPSSVSASVGDRVTITCHASQGISSNIGWLQQKPGKAP |
| | BMVL.2 | KLLIYRGTNLEDGVPSRFSGSGSGTDYTLTISSLQPEDFATYYC |
| | | VQYAQFPPTFGGGTKLEIK |
| 177 | CUSB0125- | HASQGISSNIG |
| | BMVL.2- | |
| | CDR1 | |
| 178 | CUSB0125- | RGTNLED |
| | BMVL.2- | |
| | CDR2 | |
| 179 | CUSB0125- | VQYAQFPPT |
| | BMVL.2- | |
| | CDR3 | |
| 143 | CUSB0125- | DIQMTQSPSSVSASVGDRVTITCHASQGISSNIGWLQQKPGKAF |
| | BMVL.3 | KGLIYRGTNLEDGVPSRFSGSGSGTDYTLTISSLQPEDFATYYC |
| | | VQYAQFPPTFGGGTKLEIK |
| 180 | CUSB0125- | HASQGISSNIG |
| | BMVL.3- | |
| | CDRI | |
| 181 | CUSB0125- | RGTNLED |
| | BMVL.3- | |
| | CDR2 | |
| 182 | CUSB0125- | VQYAQFPPT |
| | BMVL.3- | |
| | CDR3 | |
| 144 | CUSB0136- | QVQLQESGPGLVKPSETLSLTCTVSGFSLTTYDIIWIRQPPGKGL |
| | BMVH.1 | EWIGVIWTGGDINYNSAFMSRVTISVDTSKNQFSLKLSSVTAA |
| | | DTAVYYCARDWAYWGQGTLVTVSS |
| 183 | CUSB0136- | TYDII |
| | BMVH.1- | |
| | CDR1 | |
| 184 | CUSB0136- | VIWTGGDTNYNSAFMS |
| | BMVH.1- | |
| | CDR2 | |
| 185 | CUSB0136- | DWAY |
| | BMVH.1- | |
| | CDR3 | |
| 145 | CUSB0136- | QVQLQESGPGLVKPSETLSITCTVSGFSLTTYDIIWIRQSPGKGL |
| | BMVH.2 | EWLGVIWTGGDTNYNSAFMSRLSISKDTSKNQISLKMSSLTAA |
| | | DTAVYYCARDWAYWGQGTLVTVSS |
| 186 | CUSB0136- | TYDII |
| | BMVH.2- | |
| | CDR1 | |
| 187 | CUSB0136- | VIWTGGDTNYNSAFMS |
| | BMVH.2- | |
| | CDR2 | |
| 188 | CUSB0136- | DWAY |
| | BMVH.2- | |
| | CDR3 | |
| 146 | CUSB0136- | QVQLQESGPGLVKPSETLSLTCTVSGFSLTTYDIIWIRQSPGKGL |
| | BMVH.3 | EWLGVIWTGGDTNYNSAFMSRLTISKDTSKNQISLKMSSLTAA |
| | | DTAVYYCARDWAYWGQGTLVTVSS |
| 189 | CUSB0136- | TYDII |
| | BMVH.3- | |
| | CDR1 | |
| 190 | CUSB0136- | VIWTGGDTNYNSAFMS |
| | BMVH.3- | |
| | CDR2 | |
| 191 | CUSB0136- | DWAY |
| | BMVH.3- | |
| | CDR3 | |
| 147 | CUSB0136- | QVQLQESGPGLVKPSETLSLTCTVSGFSLTTYDIIWIRQSPGKGL |
| | BMVH.4 | EWLGVIWTGGDTNYNSAFMSRLSISKDTSKNQFSLKMSSLTAA |
| | | DTAVYYCARDWAYWGQGTLVTVSS |
| 192 | CUSB0136- | TYDII |
| | BMVH.4- | |
| | CDRI | |
| 193 | CUSB0136- | VIWTGGDTNYNSAFMS |
| | BMVH.4- | |
| | CDR2 | |
| 194 | CUSB0136- | DWAY |
| | BMVH.4- | |
| | CDR3 | |
| 148 | CUSB0136- | QVQLQESGPGLVKPSETLSLTCTVSGFSLTTYDIIWIRQSPGKGL |
| | BMVH.5 | EWLGVIWTGGDTNYNSAFMSRLTISKDTSKNQFSLKMSSLTA |
| | | ADTAVYYCARDWAYWGQGTLVTVSS |
| 195 | CUSB0136- | TYDII |
| | BMVH.5- | |
| | CDR1 | |
| 196 | CUSB0136- | VIWTGGDTNYNSAFMS |
| | BMVH.5- | |
| | CDR2 | |
| 197 | CUSB0136- | DWAY |
| | BMVH.5- | |
| | CDR3 | |
| 149 | CUSB0136- | QVQLQESGPGLVKPSETLSLTCTVSGFSLTTYDIIWIRQSPGKGL |
| | BMVH.6 | EWLGVIWTGGDTNYNSAFMSRLTISKDTSKNQFSLKMSSVTA |
| | | ADTAVYYCARDWAYWGQGTLVTVSS |
| 198 | CUSB0136- | TYDII |
| | BMVH.6- | |
| | CDR1 | |
| 199 | CUSB0136- | VIWTGGDTNYNSAFMS |
| | BMVH.6- | |
| | CDR2 | |
| 200 | CUSB0136- | DWAY |
| | BMVH.6- | |
| | CDR3 | |
| 150 | CUSB0136- | DIVMTQSPDSLAVSLGERATINCRASESVDNYGNSFIQWYQQK |
| | BMVL.1 | PGQPPKLLIYFASNLQSGVPDRFSGSGSGTDFTLTISSLQAEDVA |
| | | VYYCHQNNEDPFTFGGGTKLEIK |
| 201 | CUSB0136- | RASESVDNYGNSFIQ |
| | BMVL.1- | |
| | CDR1 | |
| 202 | CUSB0136- | FASNLQS |
| | BMVL.1- | |
| | CDR2 | |
| 203 | CUSB0136- | HQNNEDPFT |
| | BMVL.1- | |
| | CDR3 | |
| 151 | CUSB0136- | DIVLTQSPDSLAVSLGERATISCRASESVDNYGNSFIQWYQQKP |
| | BMVL.2 | GQPPKLLIYFASNLQSGVPDRFSGSGSGTDFTLTISSVQAEDVA |
| | | TYYCHQNNEDPFTFGGGTKLEIK |
| 204 | CUSB0136- | RASESVDNYGNSFIQ |
| | BMVL.2- | |
| | CDR1 | |
| 205 | CUSB0136- | FASNLQS |
| | BMVL.2- | |
| | CDR2 | |
| 206 | CUSB0136- | HQNNEDPFT |
| | BMVL.2- | |
| | CDR3 | |
| 152 | CUSB0136- | DIVLTQSPDSLAVSLGERATISCRASESVDNYGNSFIQWYQQKP |
| | BMVL.3 | GQPPKLLIYFASNLQSGVPDRFSGSGSGTDFTLTISSLQAEDVA |
| | | TYYCHQNNEDPFTFGGGTKLEIK |
| 207 | CUSB0136- | RASESVDNYGNSFIQ |
| | BMVL.3- | |
| | CDR1 | |
| 208 | CUSB0136- | FASNLQS |
| | BMVL.3- | |
| | CDR2 | |
| 209 | CUSB0136- | HQNNEDPFT |
| | BMVL.3- | |
| | CDR3 | |
| 153 | CUSB0136- | QVQLQESGPGLVKPSETLSLTCTVSGFSLTTYDIIWIRQSPGKGL |
| | BMVH.3a | EWLGVIWTGGDTWYNSAFMSRLTISKDTSKNQISLKMSSLTA |
| | | ADTAVYYCARDWAYWGQGTLVTVSS |
| 210 | CUSB0136- | TYDII |
| | BMVH.3a- | |
| | CDR1 | |
| 211 | CUSB0136- | VIWTGGDTWYNSAFMS |
| | BMVH.3a- | |
| | CDR2 | |
| 212 | CUSB0136- | DWAY |
| | BMVH.3a- | |
| | CDR3 | |
| 154 | CUSB0136- | QVQLQESGPGLVKPSETLSLTCTVSGFSLTVYDIIWIRQSPGKG |
| | BMVH.3b | LEWLGVIWTGGDTTYNSAFMSRLTISKDTSKNQISLKMSSLTA |
| | | ADTAVYYCARDWIYWGQGTLVTVSS |
| 213 | CUSB0136- | VYDII |
| | BMVH.3b- | |
| | CDR1 | |
| 214 | CUSB0136- | VIWTGGDTTYNSAFMS |
| | BMVH.3b- | |
| | CDR2 | |
| 215 | CUSB0136- | DWIY |
| | BMVH.3b- | |
| | CDR3 | |
| 155 | CUSB0136- | QVQLQESGPGLVKPSETLSLTCTVSGFSLTVYDIIWIRQSPGKG |
| | BMVH.3c | LEWLGVIWTGGDTTYNSAFMSRLTISKDTSKNQISLKMSSLTA |
| | | ADTAVYYCARDWAYWGQGTLVTVSS |
| 216 | CUSB0136- | VYDII |
| | BMVH.3c- | |
| | CDR1 | |
| 217 | CUSB0136- | VIWTGGDTTYNSAFMS |
| | BMVH.3c- | |
| | CDR2 | |
| 218 | CUSB0136- | DWAY |
| | BMVH.3c- | |
| | CDR3 | |
| 156 | CUSB0136- | QVQLQESGPGLVKPSETLSLTCTVSGFSLTVYDIIWIRQSPGKG |
| | BMVH.3d | LEWLGVIWTGGDTTYNSAFMSRLTISKDTSKNQISLKMSSLTA |
| | | ADTAVYYCARDWMYWGQGTLVTVSS |
| 219 | CUSB0136- | VYDII |
| | BMVH.3d- | |
| | CDR1 | |
| 220 | CUSB0136- | VIWTGGDTTYNSAFMS |
| | BMVH.3d- | |
| | CDR2 | |
| 221 | CUSB0136- | DWMY |
| | BMVH.3d- | |
| | CDR3 | |
| 157 | CUSB0136- | DIVLTQSPDSLAVSLGERATISCRASESVDNWGNSFIQWYQQK |
| | BMVL.2a | PGQPPKLLIYFASYLQSGVPDRFSGSGSGTDFTLTISSVQAEDV |
| | | ATYYCHQNNEDPFTFGGGTKLEIK |
| 222 | CUSB0136- | RASESVDNWGNSFIQ |
| | BMVL.2a- | |
| | CDR1 | |
| 223 | CUSB0136- | FASYLQS |
| | BMVL.2a- | |
| | CDR2 | |
| 224 | CUSB0136- | HQNNEDPFT |
| | BMVL.2a- | |
| | CDR3 | |
| 158 | CUSB0136- | DIVLTQSPDSLAVSLGERATISCRASESVDNYGNSFIQWYQQKP |
| | BMVL.2b | GQPPKLLIYFASYLQSGVPDRFSGSGSGTDFTLTISSVQAEDVA |
| | | TYYCHQNNEDPFTFGGGTKLEIK |
| 225 | CUSB0136- | RASESVDNYGNSFIQ |
| | BMVL.2b- | |
| | CDR1 | |
| 226 | CUSB0136- | FASYLQS |
| | BMVL.26- | |
| | CDR2 | |
| 227 | CUSB0136- | HQNNEDPFT |
| | BMVL.2b- | |
| | CDR3 | |
| 228 | Consensus | X₁YDII, wherein X₁ is Tor V |
| | CUSB0136 | |
| | VH-CDRI | |
| 229 | Consensus | VIWTGGDTX₂YNSAFMS, wherein X₂ is N, W or T |
| | CUSB0136 | |
| | VH-CDR2 | |
| 230 | Consensus | DWX₃Y, wherein X₃ is A, I or M |
| | CUSB0136 | |
| | VH-CDR3 | |
| 231 | Consensus | RASESVDNX₄GNSFIQ, wherein X₄ is Y or W |
| | CUSB0136 | |
| | VL-CDR1 | |
| 232 | Consensus | FASX₅LQS, wherein X₅ is N, Y, Hor Q |
| | CUSB0136 | |
| | VL-CDR2 | |
| 233 | Consensus | X₆VQLX₇ESGPGLVKPSQX₈LSLTCTVX₉GYSITSDYAWNWIRQ |
| | CUSB0125 | X₁₀PGX₁₁X₁₂LEWX₁₃GYIYYSGTTSYNPSLKSRX₁₄X₁₅IX₁₆X₁₇DTS |
| | VH | KNQFX₁₈LX₁₉LX₂₀SVTX₂₁X₂₂DTAX₂₃YYCARNYGSAFYYFDYW |
| | | GQGTTLTVSS, wherein X₆ is D or Q; X₇ is R or Q; X₈ is S or T; X₉ |
| | | is T or S; X₁₀ is F or P; X₁₁ is N or K; X₁₂ is K or G; X₁₃ is M or I; X₁₄ |
| | | is I or V; X₁₅ is S or T; X₁₆ is T or S; X₁₇ is R or V; X₁₈ is F or S; X₁₉ |
| | | is Q or K; X₂₀ is N or S; X₂₁ is T or A; X₂₂ is E or A; and/or X₁₃ is T |
| | | or V |
| 234 | Consensus | DIX₂₄MTQSPSSX₂₅SX₂₆SX₂₇GDX₂₈VTITCHASQGISSNIGWX₂₉Q |
| | CUSB0125 | QKPGKX₃₀X₃₁KX₃₂LIYRGTNLEDGVPSRFSGSGSGX₃₃DX₃₄X₃₅L |
| | VL | TISSLX₃₆X₃₇EDFAX₃₈YYCVQYAQFPPTFGGGTKLEIK, wherein |
| | | X₂₄ is L or Q; X₂₅ is M or V; X₂₆ is V or A; X₂₇ is L or V; X₂₈ is T or |
| | | R; X₂₉ is L or Y; X₃₀ is S or A; X₃₁ is F or P; X₃₂ is G or L; X₃₃ is A or |
| | | T; X₃₄ is Y or F; X₃₅ is S or T; X₃₆ is E or Q; X₃₇ is S or P; and/or X₃₈ |
| | | is D or T |
| 235 | Consensus | QVQLX₃₉ESGPGLVX₄₀PSX₄₁X₄₂LSX₄₃TCTVSGFSLTX₄₄YDIIWIR |
| | CUSB0136 | QX₄₅PGKGLEWX₄₆GVIWTGGDTX₄₇YNSAFMSRX₄₈X₄₉ISX₅₀DX₅₁ |
| | VH | SKX₅₂QX₅₃X₅₄LKX₅₅X₅₆SX₅₇X₅₈X₅₉X₆₀DTAX₆₁YYCX₆₂RDWX₆₃Y |
| | | WGQGTLVTVSX₆₄, wherein X₃₉ is K or Q; X₄₀ is A or K; X₄₁ is Q |
| | | or E; X₄₂ is S or T; X₄₃ is I or L; X₄₄ is T or V; X₄₅ is S or P; X₄₆ is L |
| | | or I; X₄₇ is N, W or T; X₄₈ is L or V; X₄₉ is T or S; X₅₀ is K or V; X₅₁ |
| | | is N or T; X₅₂ is S or N; X₅₃ is I or F; X₅₄ is F or S; X₅₅ is M or L; X₅₆ |
| | | is N or S; X₅₇ is L or V; X₅₈ is Q or T; X₅₉ is T or A; X₆₀ is D or A; |
| | | X₆₁ is I or V; X₆₂ is A or V; X₆₃ is A, I or M; and/or X₆₄ is T or S |
| 236 | Consensus | X₆₅IVX₆₆TQSPX₆₇SLAVSLGX₆₈GRATIX₆₉CRASESVDNX₇₀GNSFIQ |
| | CUSB0136 | WYQQKPGQPPKLLIYFASX₇₁LQSGVPX₇₂RFSGSGSX₇₃TDFTLTI |
| | VL | X₇₄X₇₅X₇₆X₇₇AX₇₈DX₇₉AX₈₀YYCHQNNEDPFTFGX₈₁GTKLEIK, |
| | | wherein X₆₅ is N or D; X₆₆ is L or M; X₆₇ is A or D; X₆₈ is Q or E; X₆₉ |
| | | is S or N; X₇₀ is Y or W; X₇₁ is N or Y; X₇₂ is A or D; X₇₃ is R or G; |
| | | X₇₄ is D or S; X₇₅ is P or S; X₇₆ is V or L; X₇₇ is E or Q; X₇₈ is D or E; |
| | | X₇₉ is A or V; X₈₀ is T or V; and/or X81 is S or G |

In some embodiments, an anti-PD-1 antibody or antigen-binding fragment thereof comprises a VH comprising a CDR1 comprising the sequence set forth in SEQ ID NO: 81, a CDR2 comprising the sequence set forth in SEQ ID NO: 82, and/or a CDR3 comprising the sequence set forth in SEQ ID NO: 83.

In some embodiments, an anti-PD-1 antibody or antigen-binding fragment thereof comprises a VH comprising a CDR1 comprising the sequence set forth in SEQ ID NO: 89, a CDR2 comprising the sequence set forth in SEQ ID NO: 90, and/or a CDR3 comprising the sequence set forth in SEQ ID NO: 91.

In some embodiments, an anti-PD-1 antibody or antigen-binding fragment thereof comprises a VH comprising a CDR1 comprising the sequence set forth in SEQ ID NO: 97, a CDR2 comprising the sequence set forth in SEQ ID NO: 98, and/or a CDR3 comprising the sequence set forth in SEQ ID NO: 99.

In some embodiments, an anti-PD-1 antibody or antigen-binding fragment thereof comprises a VH comprising a CDR1 comprising the sequence set forth in SEQ ID NO: 105, a CDR2 comprising the sequence set forth in SEQ ID NO: 106, and/or a CDR3 comprising the sequence set forth in SEQ ID NO: 107.

In some embodiments, an anti-PD-1 antibody or antigen-binding fragment thereof comprises a VH comprising a CDR1 comprising the sequence set forth in SEQ ID NO: 113, a CDR2 comprising the sequence set forth in SEQ ID NO: 114, and/or a CDR3 comprising the sequence set forth in SEQ ID NO: 115.

In some embodiments, an anti-PD-1 antibody or antigen-binding fragment thereof comprises a VH comprising a CDR1 comprising the sequence set forth in SEQ ID NO: 121, a CDR2 comprising the sequence set forth in SEQ ID NO: 122, and/or a CDR3 comprising the sequence set forth in SEQ ID NO: 123.

In some embodiments, an anti-PD-1 antibody or antigen-binding fragment thereof comprises a VH comprising a CDR1 comprising the sequence set forth in SEQ ID NO: 129, a CDR2 comprising the sequence set forth in SEQ ID NO: 130, and/or a CDR3 comprising the sequence set forth in SEQ ID NO: 131.

In some embodiments, an anti-PD-1 antibody or antigen-binding fragment thereof comprises a VH comprising a CDR1 comprising the sequence X₁YDII (SEQ ID NO: 228), wherein X₁ is T or V, a CDR2 comprising the sequence VIWTGGDTX₂YNSAFMS (SEQ ID NO: 229), wherein X₂ is N, W or T, and/or a CDR3 comprising the sequence DWX₃Y (SEQ ID NO: 230), wherein X₃ is A, I or M.

In some embodiments, an anti-PD-1 antibody or antigen-binding fragment thereof comprises a VL comprising a CDR1 comprising the sequence set forth in SEQ ID NO: 85, a CDR2 comprising the sequence set forth in SEQ ID NO: 86, and/or a CDR3 comprising the sequence set forth in SEQ ID NO: 87.

In some embodiments, an anti-PD-1 antibody or antigen-binding fragment thereof comprises a VL comprising a CDR1 comprising the sequence set forth in SEQ ID NO: 93, a CDR2 comprising the sequence set forth in SEQ ID NO: 94, and/or a CDR3 comprising the sequence set forth in SEQ ID NO: 95.

In some embodiments, an anti-PD-1 antibody or antigen-binding fragment thereof comprises a VL comprising a CDR1 comprising the sequence set forth in SEQ ID NO: 101, a CDR2 comprising the sequence set forth in SEQ ID NO: 102, and/or a CDR3 comprising the sequence set forth in SEQ ID NO: 103.

In some embodiments, an anti-PD-1 antibody or antigen-binding fragment thereof comprises a VL comprising a CDR1 comprising the sequence set forth in SEQ ID NO: 109, a CDR2 comprising the sequence set forth in SEQ ID NO: 110, and/or a CDR3 comprising the sequence set forth in SEQ ID NO: 111.

In some embodiments, an anti-PD-1 antibody or antigen-binding fragment thereof comprises a VL comprising a CDR1 comprising the sequence set forth in SEQ ID NO: 117, a CDR2 comprising the sequence set forth in SEQ ID NO: 118, and/or a CDR3 comprising the sequence set forth in SEQ ID NO: 119.

In some embodiments, an anti-PD-1 antibody or antigen-binding fragment thereof comprises a VL comprising a CDR1 comprising the sequence set forth in SEQ ID NO: 125, a CDR2 comprising the sequence set forth in SEQ ID NO: 126, and/or a CDR3 comprising the sequence set forth in SEQ ID NO: 127.

In some embodiments, an anti-PD-1 antibody or antigen-binding fragment thereof comprises a VL comprising a CDR1 comprising the sequence set forth in SEQ ID NO: 133, a CDR2 comprising the sequence set forth in SEQ ID NO: 134, and/or a CDR3 comprising the sequence set forth in SEQ ID NO: 135.

In some embodiments, an anti-PD-1 antibody or antigen-binding fragment thereof comprises a VL comprising a CDR1 comprising the sequence RASESVDNX₄GNSFIQ (SEQ ID NO: 231), wherein X₄ is Y or W, a CDR2 comprising the sequence FASX₅LQS (SEQ ID NO: 232), wherein X₅ is N, Y, H or Q. and/or a CDR3 comprising the sequence HQNNEDPFT (SEQ ID NO: 227).

In some embodiments, an anti-PD-1 antibody or antigen-binding fragment thereof comprises (a) a VH comprising a CDR1 comprising the sequence set forth in SEQ ID NO: 81, a CDR2 comprising the sequence set forth in SEQ ID NO: 82, and a CDR3 comprising the sequence set forth in SEQ ID NO: 83; and (b) a VL comprising a CDR1 comprising the sequence set forth in SEQ ID NO: 85, a CDR2 comprising the sequence set forth in SEQ ID NO: 86, and a CDR3 comprising the sequence set forth in SEQ ID NO: 87.

In some embodiments, an anti-PD-1 antibody or antigen-binding fragment thereof comprising (a) a VH comprising a CDR1 comprising the sequence set forth in SEQ ID NO: 89, a CDR2 comprising the sequence set forth in SEQ ID NO: 90, and a CDR3 comprising the sequence set forth in SEQ ID NO: 91; and (b) a VL comprising a CDR1 comprising the sequence set forth in SEQ ID NO: 93, a CDR2 comprising the sequence set forth in SEQ ID NO: 94, and a CDR3 comprising the sequence set forth in SEQ ID NO: 95.

In some embodiments, an anti-PD-1 antibody or antigen-binding fragment thereof comprising (a) a VH comprising a CDR1 comprising the sequence set forth in SEQ ID NO: 97, a CDR2 comprising the sequence set forth in SEQ ID NO: 98, and a CDR3 comprising the sequence set forth in SEQ ID NO: 99; and (b) a VL comprising a CDR1 comprising the sequence set forth in SEQ ID NO: 101, a CDR2 comprising the sequence set forth in SEQ ID NO: 102, and a CDR3 comprising the sequence set forth in SEQ ID NO: 103.

In some embodiments, an anti-PD-1 antibody or antigen-binding fragment thereof comprising (a) a VH comprising a CDR1 comprising the sequence set forth in SEQ ID NO: 105, a CDR2 comprising the sequence set forth in SEQ ID NO: 106, and a CDR3 comprising the sequence set forth in SEQ ID NO: 107; and (b) a VL comprising a CDR1 comprising the sequence set forth in SEQ ID NO: 109, a CDR2 comprising the sequence set forth in SEQ ID NO: 110, and a CDR3 comprising the sequence set forth in SEQ ID NO: 111.

In some embodiments, an anti-PD-1 antibody or antigen-binding fragment thereof comprising (a) a VH comprising a CDR1 comprising the sequence set forth in SEQ ID NO: 113, a CDR2 comprising the sequence set forth in SEQ ID NO: 114, and a CDR3 comprising the sequence set forth in SEQ ID NO: 115; and (b) a VL comprising a CDR1 comprising the sequence set forth in SEQ ID NO: 117, a CDR2 comprising the sequence set forth in SEQ ID NO: 118, and a CDR3 comprising the sequence set forth in SEQ ID NO: 119.

In some embodiments, an anti-PD-1 antibody or antigen-binding fragment thereof comprising (a) a VH comprising a CDR1 comprising the sequence set forth in SEQ ID NO: 121, a CDR2 comprising the sequence set forth in SEQ ID NO: 122, and a CDR3 comprising the sequence set forth in SEQ ID NO: 123; and (b) a VL comprising a CDR1 comprising the sequence set forth in SEQ ID NO: 125, a CDR2 comprising the sequence set forth in SEQ ID NO: 126, and a CDR3 comprising the sequence set forth in SEQ ID NO: 127.

In some embodiments, an anti-PD-1 antibody or antigen-binding fragment thereof comprising (a) a VH comprising a CDR1 comprising the sequence set forth in SEQ ID NO: 129, a CDR2 comprising the sequence set forth in SEQ ID NO: 130, and a CDR3 comprising the sequence set forth in SEQ ID NO: 131; and (b) a VL comprising a CDR1 comprising the sequence set forth in SEQ ID NO: 133, a CDR2 comprising the sequence set forth in SEQ ID NO: 134, and a CDR3 comprising the sequence set forth in SEQ ID NO: 135.

In some embodiments, an anti-PD-1 antibody or antigen-binding fragment thereof comprises (a) a VH comprising a CDR1 comprising the sequence X₁YDII (SEQ ID NO: 228), wherein X₁ is T or V, a CDR2 comprising the sequence VIWTGGDTX₂YNSAFMS (SEQ ID NO: 229), wherein X₂ is N, W or T, and a CDR3 comprising the sequence DWX₃Y (SEQ ID NO: 230), wherein X₃ is A, I or M; and (b) a VL comprising a CDR1 comprising the sequence RASESVDNX₄GNSFIQ (SEQ ID NO: 231), wherein X₄ is Y or W, a CDR2 comprising the sequence FASX₅LQS (SEQ ID NO: 232), wherein X₅ is N, Y, H or Q, and a CDR3 comprising the sequence HQNNEDPFT (SEQ ID NO: 227).

In some embodiments, an anti-PD-1 antibody or antigen-binding fragment thereof comprises a VH CDR1, VH CDR2, VH CDR3, VL CDR1, VL CDR2 and/or VL CDR3 set forth in

Table 1 with one or more conservative amino acid substitutions.

A “conservative amino acid substitution” as used herein, is one in which one amino acid residue is replaced with another amino acid residue are substitutions that change an amino acid to a different amino acid with similar biochemical properties (e.g. charge, hydrophobicity and size). For example, lysine, arginine and histidine have similar properties in that they have a basic side-chain, and aspartic acid and glutamic acid have similar properties in that they have an acidic side chain. In addition, glycine, asparagine, glutamine, serine, threonine, tyrosine, cysteine and tryptophan have similar properties in that they have an uncharged polar side-chain, and alanine, valine, leucine, threonine, isoleucine, proline, phenylalanine and methionine have similar properties in that they have a non-polar side-chain. Also, tyrosine, phenylalanine, tryptophan and histidine have similar properties in that they have an aromatic side-chain. Thus, it will be obvious to those skilled in the art that, even when substitution of amino acid residues in groups showing similar properties as described above occurs; it will show no particular change in the properties.

In some aspects, an anti-PD-1 antibody provided herein comprises a VH and/or a VL set forth in

Table 1. In some embodiments, an anti-PD-1 antibody provided herein comprises a VH and/or a VL that is at least 80%, at least 85%, at least 90%, at least 95%, at least 96%, at least 97%, at least 98%, or at least 99% identical to a VH and/or VL, respectively, set forth in Table 1.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul. 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, an anti-PD-1 antibody provided herein comprises a VH sequence set forth in

Table 1 with one or more (e.g., 1, 2, 3, 4, 5, 6, 7, 8, 9 or 10) conservative amino acid substitutions. In some embodiments, an anti-PD-1 antibody provided herein comprises a VL sequence set forth in
Table 1 with one or more (e.g., 1, 2, 3, 4, 5, 6, 7, 8, 9 or 10) conservative amino acid substitutions.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 80. In some embodiments, an anti-PD-1 antibody or antigen-binding fragment thereof comprises a VH comprising the sequence set forth in SEQ ID NO: 80.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 88. In some embodiments, an anti-PD-1 antibody or antigen-binding fragment thereof comprises a VH comprising the sequence set forth in SEQ ID NO: 88.

In some embodiments, an anti-PD-1 antibody or antigen-binding fragment thereof comprises a VH comprising a sequence that is at least 80%, at least 85%, at least 900, at least 95%, at least 96%, at least 97%, at least 98%, or at least 99% identical to the sequence set forth in SEQ ID NO: 96. In some embodiments, an anti-PD-1 antibody or antigen-binding fragment thereof comprises a VH comprising the sequence set forth in SEQ ID NO: 96.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 104. In some embodiments, an anti-PD-1 antibody or antigen-binding fragment thereof comprises a VH comprising the sequence set forth in SEQ ID NO: 104.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 112. In some embodiments, an anti-PD-1 antibody or antigen-binding fragment thereof comprises a VH comprising the sequence set forth in SEQ ID NO: 112.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 120. In some embodiments, an anti-PD-1 antibody or antigen-binding fragment thereof comprises a VH comprising the sequence set forth in SEQ ID NO: 120.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 128. In some embodiments, an anti-PD-1 antibody or antigen-binding fragment thereof comprises a VH comprising the sequence set forth in SEQ ID NO: 128.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 136. In some embodiments, an anti-PD-1 antibody or antigen-binding fragment thereof comprises a VH comprising the sequence set forth in SEQ ID NO: 136.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 137. In some embodiments, an anti-PD-1 antibody or antigen-binding fragment thereof comprises a VH comprising the sequence set forth in SEQ ID NO: 137.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 138. In some embodiments, an anti-PD-1 antibody or antigen-binding fragment thereof comprises a VH comprising the sequence set forth in SEQ ID NO: 138.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 139. In some embodiments, an anti-PD-1 antibody or antigen-binding fragment thereof comprises a VH comprising the sequence set forth in SEQ ID NO: 139.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 140. In some embodiments, an anti-PD-1 antibody or antigen-binding fragment thereof comprises a VH comprising the sequence set forth in SEQ ID NO: 140.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 144. In some embodiments, an anti-PD-1 antibody or antigen-binding fragment thereof comprises a VH comprising the sequence set forth in SEQ ID NO: 144.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 145. In some embodiments, an anti-PD-1 antibody or antigen-binding fragment thereof comprises a VH comprising the sequence set forth in SEQ ID NO: 145.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 146. In some embodiments, an anti-PD-1 antibody or antigen-binding fragment thereof comprises a VH comprising the sequence set forth in SEQ ID NO: 146.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 147. In some embodiments, an anti-PD-1 antibody or antigen-binding fragment thereof comprises a VH comprising the sequence set forth in SEQ ID NO: 147.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 148. In some embodiments, an anti-PD-1 antibody or antigen-binding fragment thereof comprises a VH comprising the sequence set forth in SEQ ID NO: 148.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 149. In some embodiments, an anti-PD-1 antibody or antigen-binding fragment thereof comprises a VH comprising the sequence set forth in SEQ ID NO: 149.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 153. In some embodiments, an anti-PD-1 antibody or antigen-binding fragment thereof comprises a VH comprising the sequence set forth in SEQ ID NO: 153.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 154. In some embodiments, an anti-PD-1 antibody or antigen-binding fragment thereof comprises a VH comprising the sequence set forth in SEQ ID NO: 154.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 155. In some embodiments, an anti-PD-1 antibody or antigen-binding fragment thereof comprises a VH comprising the sequence set forth in SEQ ID NO: 155.

In some embodiments, an anti-PD-1 antibody or antigen-binding fragment thereof comprises a VH comprising a sequence that is at least 80%, at least 85%, at least 90%, at least 95%, at least 96%, at least 97%, at least 98%, or at least 99% identical to the sequence set forth in SEQ ID NO: 156. In some embodiments, an anti-PD-1 antibody or antigen-binding fragment thereof comprises a VH comprising the sequence set forth in SEQ ID NO: 156.

In some embodiments, an anti-PD-1 antibody or antigen-binding fragment thereof comprises a VH comprising the sequence X₆VQLX₇ESGPGLVKPSQX₈LSLTCTVX₉GYSITSDYAWNWIRQX₁₀PGX₁₁X₁₂LEWX₁₃G YIYYSGTISYNPSLKSRX₁₄X₁₅IX₁₆X₁₇DTSKNQFX₁₈LX₁₉LX₂₀SVTX₂₁X₂₂DTAX₂₃YYCA RNYGSAFYYFDYWGQGTTLTVSS, wherein X₆ is D or Q; X₇ is R or Q; X₈ is S or T; X₉ is T or S; X₁₀ is F or P; X₁₁ is N or K; X₁₂ is K or G; X₁₃ is M or I; X₁₄ is I or V; X₁₅ is S or T; X₁₆ is T or S; X₁₇ is R or V; X₁₈ is F or S; X₁₉ is Q or K; X₂₀ is N or S; X₂₁ is T or A; X₂₂ is E or A; and/or X₂₃ is T or V.

In some embodiments, an anti-PD-1 antibody or antigen-binding fragment thereof comprises a VH comprising the sequence QVQLX₃₉ESGPGLVX₄₀PSX₄₁X₄₂LSX₄₃TCTVSGFSLTX₄₄YDIIWIRQX₄₅PGKGLEWX₄₆G VIWTGGDTX₄₇YNSAFMSRX₄₈X₄₉SX₅₀DX₅₁SKX₅₂QX₅₃X₅₄LKX₅₅X₅₆SX₅₇X₅₈X₅₉X₆₀DT AX₆₁YYCX₆₂RDWX₆₃YWGQGTLVTVSX₆₄, wherein X₃₉ is K or Q; X₄₀ is A or K; X₄₁ is Q or E; X₄₂ is S or T; X₄₃ is I or L; X₄₄ is T or V; X₄₅ is S or P; X₄₆ is L or I; X₄₇ is N, W or T; X₄₉ is L or V; X₄₉ is T or S; X₅₀ is K or V; X₅₁ is N or T; X₅₂ is S or N; X₅₃ is I or F; X₅₄ is F or S; X₅₅ is M or L; X₅₆ is N or S; X₅₇ is L or V; X₅₈ is Q or T; X₅₉ is T or A; X₆₀ is D or A; X₆₁ is I or V; X₆₂ is A or V; X₆₃ is A, I or M; and/or X₆₄ is T or S.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 84. In some embodiments, an anti-PD-1 antibody or antigen-binding fragment thereof comprises a VL comprising the sequence set forth in SEQ ID NO: 84.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 92. In some embodiments, an anti-PD-1 antibody or antigen-binding fragment thereof comprises a VL comprising the sequence set forth in SEQ ID NO: 92.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99;% identical to the sequence set forth in SEQ ID NO: 100. In some embodiments, an anti-PD-1 antibody or antigen-binding fragment thereof comprises a VL comprising the sequence set forth in SEQ ID NO: 100.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 108. In some embodiments, an anti-PD-1 antibody or antigen-binding fragment thereof comprises a VL comprising the sequence set forth in SEQ ID NO: 108.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 116. In some embodiments, an anti-PD-1 antibody or antigen-binding fragment thereof comprises a VL comprising the sequence set forth in SEQ ID NO: 116.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 124. In some embodiments, an anti-PD-1 antibody or antigen-binding fragment thereof comprises a VL comprising the sequence set forth in SEQ ID NO: 124.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 132. In some embodiments, an anti-PD-1 antibody or antigen-binding fragment thereof comprises a VL comprising the sequence set forth in SEQ ID NO: 132.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 141. In some embodiments, an anti-PD-1 antibody or antigen-binding fragment thereof comprises a VL comprising the sequence set forth in SEQ ID NO: 141.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 142. In some embodiments, an anti-PD-1 antibody or antigen-binding fragment thereof comprises a VL comprising the sequence set forth in SEQ ID NO: 142.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 143. In some embodiments, an anti-PD-1 antibody or antigen-binding fragment thereof comprises a VL comprising the sequence set forth in SEQ ID NO: 143.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 150. In some embodiments, an anti-PD-1 antibody or antigen-binding fragment thereof comprises a VL comprising the sequence set forth in SEQ ID NO: 150.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 151. In some embodiments, an anti-PD-1 antibody or antigen-binding fragment thereof comprises a VL comprising the sequence set forth in SEQ ID NO: 151.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 152. In some embodiments, an anti-PD-1 antibody or antigen-binding fragment thereof comprises a VL comprising the sequence set forth in SEQ ID NO: 152.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 157. In some embodiments, an anti-PD-1 antibody or antigen-binding fragment thereof comprises a VL comprising the sequence set forth in SEQ ID NO: 157.

In some embodiments, an anti-PD-1 antibody or antigen-binding fragment thereof comprises a VL comprising a sequence that is at least 80%, at least 85%, at least 90%, at least 95%, at least 96%, at least 97%, at least 98%, or at least 99% identical to the sequence set forth in SEQ ID NO: 158. In some embodiments, an anti-PD-1 antibody or antigen-binding fragment thereof comprises a VL comprising the sequence set forth in SEQ ID NO: 158.

In some embodiments, an anti-PD-1 antibody or antigen-binding fragment thereof comprises a VL comprising the sequence DIX₂₄MTQSPSSX₂₅SX₂₆SX₂₇GDX₂₈VTITCHASQGISSNIGWX₂₉QQKPGKX₃₀X₃₁KX₃₂LI YRGTNLEDGVPSRFSGSGSGX₃₃DX₃₄X₃₅LTISSLX₃₆X₃₇EDFAX₃₈YYCVQYAQFPPTFG GGTKLEIK, wherein X₂₄ is L or Q; X₂₅ is M or V; X₂₆ is V or A; X₂₇ is L or V; X₂₈ is T or R; X₂₉ is L or Y; X₃₀ is S or A; X₃₁ is F or P; X₃₂ is G or L; X₃₃ is A or T; X₃₄ is Y or F; X₃₅ is S or T; X₃₆ is E or Q; X₃₇ is S or P; and/or X₃₈ is D or T.

In some embodiments, an anti-PD-1 antibody or antigen-binding fragment thereof comprises a VL comprising the sequence X₆₃IVX₆₆TQSPX₆₇SLAVSLGX₆₈RATIX₆₉CRASESVDNX₇₀GNSFIQWYQQKPGQPPKLLI YFASX₇₁LQSGVPX₇₂RFSGSGSX₇₃TDFTLTIX₇₄X₇₅X₇₆X₇₇AX₇₈DX₇₉AX₈₀YYCHQNNED PFTFGX₈₁GTKLEIK, wherein X₆₅ is N or D; X₆₆ is L or M; X₆₇ is A or D; X₆₈ is Q or E; X₆₉ is S or N; X₇₀ is Y or W; X₇₁ is N or Y; X₇₂ is A or D; X₇₃ is R or G; X₇₄ is D or S; X₇₅ is P or S; X₇₆ is V or L; X₇₇ is E or Q; X₇₈ is D or E; X₇₉ is A or V; X₈₀ is T or V; and/or X₈₁ is S or G.

It will be apparent to a person of skill in the art that the variable and light chains of different antibodies may be freely combined. Thus, for example, an anti-PD-1 antibody may comprise the VH of the antibody with the description “CUSB0103” in Table 1 and the VL of the antibody with the description “CUSB0123” in Table 1.

In some embodiments, an anti-PD-1 antibody or antigen-binding fragment thereof comprises a VH comprising the sequence set forth in SEQ ID NO: 80 and a VL comprising the sequence set forth in SEQ ID NO: 84.

In some embodiments, an anti-PD-1 antibody or antigen-binding fragment thereof comprises a VH comprising the sequence set forth in SEQ ID NO: 88 and a VL comprising the sequence set forth in SEQ ID NO: 92.

In some embodiments, an anti-PD-1 antibody or antigen-binding fragment thereof comprises a VH comprising the sequence set forth in SEQ ID NO: 96 and a VL comprising the sequence set forth in SEQ ID NO: 100.

In some embodiments, an anti-PD-1 antibody or antigen-binding fragment thereof comprises a VH comprising the sequence set forth in SEQ ID NO: 104 and a VL comprising the sequence set forth in SEQ ID NO: 108.

In some embodiments, an anti-PD-1 antibody or antigen-binding fragment thereof comprises a VH comprising the sequence set forth in SEQ ID NO: 112 and a VL comprising the sequence set forth in SEQ ID NO: 116.

In some embodiments, an anti-PD-1 antibody or antigen-binding fragment thereof comprises a VH comprising the sequence set forth in SEQ ID NO: 120 and a VL comprising the sequence set forth in SEQ ID NO: 124.

In some embodiments, an anti-PD-1 antibody or antigen-binding fragment thereof comprises a VH comprising the sequence set forth in SEQ ID NO: 128 and a VL comprising the sequence set forth in SEQ ID NO: 132.

In some embodiments, an anti-PD-1 antibody or antigen-binding fragment thereof comprises a VH comprising the sequence set forth in SEQ ID NO: 136 and a VL comprising the sequence set forth in SEQ ID NO: 143.

In some embodiments, an anti-PD-1 antibody or antigen-binding fragment thereof comprises a VH comprising the sequence set forth in SEQ ID NO: 137 and a VL comprising the sequence set forth in SEQ ID NO: 143.

In some embodiments, an anti-PD-1 antibody or antigen-binding fragment thereof comprises a VH comprising the sequence set forth in SEQ ID NO: 138 and a VL comprising the sequence set forth in SEQ ID NO: 143.

In some embodiments, an anti-PD-1 antibody or antigen-binding fragment thereof comprises a VH comprising the sequence set forth in SEQ ID NO: 139 and a VL comprising the sequence set forth in SEQ ID NO: 143.

In some embodiments, an anti-PD-1 antibody or antigen-binding fragment thereof comprises a VH comprising the sequence set forth in SEQ ID NO: 145 and a VL comprising the sequence set forth in SEQ ID NO: 150.

In some embodiments, an anti-PD-1 antibody or antigen-binding fragment thereof comprises a VH comprising the sequence set forth in SEQ ID NO: 145 and a VL comprising the sequence set forth in SEQ ID NO: 151.

In some embodiments, an anti-PD-1 antibody or antigen-binding fragment thereof comprises a VH comprising the sequence set forth in SEQ ID NO: 145 and a VL comprising the sequence set forth in SEQ ID NO: 152.

In some embodiments, an anti-PD-1 antibody or antigen-binding fragment thereof comprises a VH comprising the sequence set forth in SEQ ID NO: 146 and a VL comprising the sequence set forth in SEQ ID NO: 150.

In some embodiments, an anti-PD-1 antibody or antigen-binding fragment thereof comprises a VH comprising the sequence set forth in SEQ ID NO: 146 and a VL comprising the sequence set forth in SEQ ID NO: 151.

In some embodiments, an anti-PD-1 antibody or antigen-binding fragment thereof comprises a VH comprising the sequence set forth in SEQ ID NO: 146 and a VL comprising the sequence set forth in SEQ ID NO: 152.

In some embodiments, an anti-PD-1 antibody or antigen-binding fragment thereof comprises a VH comprising the sequence set forth in SEQ ID NO: 153 and a VL comprising the sequence set forth in SEQ ID NO: 157.

In some embodiments, an anti-PD-1 antibody or antigen-binding fragment thereof comprises a VH comprising the sequence set forth in SEQ ID NO: 154 and a VL comprising the sequence set forth in SEQ ID NO: 158.

In some embodiments, an anti-PD-1 antibody or antigen-binding fragment thereof comprises a VH comprising the sequence set forth in SEQ ID NO: 155 and a VL comprising the sequence set forth in SEQ ID NO: 158.

In some embodiments, an anti-PD-1 antibody or antigen-binding fragment thereof comprises a VH comprising the sequence set forth in SEQ ID NO: 156 and a VL comprising the sequence set forth in SEQ ID NO: 158.

In some embodiments, an anti-PD-1 antibody or antigen-binding fragment thereof comprises (a) a VH comprising the sequence X₆VQLX₇ESGPGLVKPSQX₈LSLTCTVX₉GYSITSDYAWNWIRQX₁₀PGX₁₁X₁₂LEWX₁₃G YIYYSGTITSYNPSLKSRX₁₄X₁₅X₁₆X₁₇DTSKNQFX₁₈LX₁₉LX₂₀SVTX₂₁X₂₂DTAX₂₃YYCA RNYGSAFYYFDYWGQGTTLTVSS, wherein X₆ is D or Q; X₇ is R or Q; X₈ is S or T; X₉ is T or S; X₁₀ is F or P; X₁₁ is N or K; X₁₂ is K or G; X₁₃ is M or I; X₁₄ is I or V; X₁₅ is Sor T; X₁₆ is T or S; X₁₇ is R or V; X₁₈ is F or S; X₁₉ is Q or K; X₂₀ is N or S; X₂₁ is T or A; X₂₂ is E or A; and X₂₃ is T or V; and (b) a VL comprising the sequence DIX₂₄MTQSPSSX₂₅SX₂₆SX₂₇GDX₂₈VTITCHASQGISSNIGWX₂₉QQKPGKX₃₀X₃₁KX₃₂LI YRGTNLEDGVPSRFSGSGSGX₃₃DX₃₄X₃₅LTISSLX₃₆X₃₇EDFAX₃₈YYCVQYAQFPPTFG GGTKLEIK, wherein X₂₄ is L or Q; X₂₅ is M or V; X₂₆ is V or A; X₂₇ is L or V; X₂₈ is T or R; X₂₉ is L or Y; X₃₀ is S or A; X₃₁ is F or P; X₃₂ is G or L; X₃₃ is A or T; X₃₄ is Y or F; X₃₅ is S or T; X₃₆ is E or Q; X₃₇ is S or P; and X₃₈ is D or T.

In some embodiments, an anti-PD-1 antibody or antigen-binding fragment thereof comprises (a) a VH comprising the sequence QVQLX₃₉ESGPGLVX₄₀PSX₄₁X₄₂LSX₄₃TCTVSGFSLTX₄₄YDIIWIRQX₄₅PGKGLEWX₄₆G VIWTGGDTX₄₇YNSAFMSRX₄₈X₄₉ISX₅₀DX₅₁SKX₅₂QX₅₃X₅₄LKX₅₅X₅₆SX₅₇X₅₈X₅₉X₆₀DT AX₆₁YYCX₆₂RDWX₆₃YWGQGTLVTVSX₆₄, wherein X₃₉ is K or Q; X₄₀ is A or K; X₄₁ is Q or E; X₄₂ is S or T; X₄₃ is I or L; X₄₄ is T or V; X₄₅ is S or P; X₄₆ is L or I; X₄₇ is N, W or T; X₄₈ is L or V; X₄₉ is T or S; X₅₀ is K or V; X₅₁ is N or T; X₅₂ is S or N; X₅₃ is I or F; X₅₄ is F or S; X₅₅ is M or L; X₅₆ is N or S; X₅₇ is L or V; X₅₈ is Q or T; X₅₉ is T or A; X₆₀ is D or A; X₆₁ is I or V; X₆₂ is A or V; X₆₃ is A, I or M; and X₆₄ is T or S; and (b) a VL comprising the sequence X₆₅IVX₆₆TQSPX₆₇SLAVSLGX₆₈RATIX₆₉CRASESVDNX₇₀GNSFIQWYQQKPGQPPKLLI YFASX₇₁LQSGVPX₇₂RFSGSGSX₇₃TDFTLTIX₇₄X₇₅X₇₆X₇₇AX₇₈DX₇₉AX₈₀YYCHQNNED PFTFGX₈₁GTKLEIK, wherein X₆₅ is N or D; X₆₆ is L or M; X₆₇ is A or D; X₆₈ is Q or E; X₆₉ is S or N; X₇₀ is Y or W; X₇₁ is N or Y; X₇₂ is A or D; X₇₃ is R or G; X₇₄ is D or S; X₇₅ is P or S; X₇₆ is V or L; X₇₇ is E or Q; X₇₈ is D or E; X₇₉ is A or V; X₈₀ is T or V; and X₈₁ is S or G.

An anti-PD-1 antibody or antigen-binding fragment thereof may be of any class of immunoglobulin. In some embodiments, an anti-PD-1 antibody or antigen-binding fragment thereof is an IgG antibody or antigen-binding fragment thereof. In some embodiments, an anti-PD-1 antibody or antigen-binding fragment thereof is an IgM antibody or antigen-binding fragment thereof. In some embodiments, an anti-PD-1 antibody or antigen-binding fragment thereof is an IgA antibody or antigen-binding fragment thereof. In some embodiments, an anti-PD-1 antibody or antigen-binding fragment thereof is an IgE antibody or antigen-binding fragment thereof. In some embodiments, an anti-PD-1 antibody or antigen-binding fragment thereof is an IgD antibody or antigen-binding fragment thereof.

An anti-PD-1 antibody or antigen-binding fragment thereof may be of any subclass of immunoglobulin. Thus, in some embodiments, an anti-PD-1 antibody or antigen-binding fragment thereof is an IgG1 antibody or antigen-binding fragment thereof, an IgG2 antibody or antigen-binding fragment thereof, an IgG3 antibody or antigen-binding fragment thereof, or an IgG4 antibody or antigen-binding fragment thereof. In some embodiments, the light chain of an anti-PD-1 antibody or antigen-binding fragment thereof is a kappa chain. In some embodiments, the light chain of an anti-PD-1 antibody or antigen-binding fragment thereof is a lambda chain.

In some embodiments, an anti-PD-1 antibody or antigen-binding fragment thereof is a monoclonal antibody or antigen-binding fragment thereof. The term “monoclonal antibody” (MAb or mAb), as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. Monoclonal antibodies contain an antigen binding site capable of immunoreacting with a particular epitope of the antigen characterized by a unique binding affinity for it.

In some embodiments, an anti-PD-1 antibody or antigen-binding fragment thereof is a humanized antibody or antigen-binding fragment thereof. Humanized antibodies may be generated by replacing the residues in the framework region of a non-human (e.g., murine) antibody with amino acid residues of the human framework regions.

In some embodiments, an anti-PD-1 antibody or antigen-binding fragment thereof is a human antibody or antigen-binding fragment thereof. Human antibodies can be prepared by administering an antigen to a transgenic animal that has been modified to produce such antibodies in response to antigenic challenge, but whose endogenous loci have been disabled. In some embodiments, an anti-PD-1 antibody or antigen-binding fragment thereof is a murine antibody or antigen-binding fragment thereof.

In some embodiments, an anti-PD-1 antibody or antigen-binding fragment thereof is a chimeric antibody or antigen-binding fragment thereof.

In some embodiments, an anti-PD-1 antibody provided herein binds to PD-1 with high affinity. Non-covalent interactions occur between an immunoglobulin molecule and an antigen for which the immunoglobulin is specific. The strength, or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (kₒₙ) and the “off rate constant” (k_{off}) can be determined by calculation of the concentrations and the actual rates of association and dissociation. (See Nature 361:186-87 (1993)). The ratio of k_{off}/kₒₙ enables the cancellation of all parameters not related to affinity, and is equal to the dissociation constant K_{D} (See, generally, Davies et al., (1990) Annual Rev Biochem 59:439-473). An antibody disclosed herein is said to specifically bind to PD-1 when the equilibrium binding constant (K_{D}) is ≤1 micromolar, ≤100 nM, ≤10 nM or ≤100 pM to about 1 pM, as measured by assays such as radioligand binding assays or similar assays known to those skilled in the art or described herein (e.g., in the Examples).

In some embodiments, an anti-PD-1 antibody provided herein binds to PD-1 with a K_{D} of less than 10 nM, less than 5 nM, less than 1 nM, less than 0.5 nM, less than 0.4 nM, less than 0.3 nM, less than 0.2 nM, less than 0.1 nM, less than 0.05 nM, less than 0.02 nM, or less than 0.01 nM. In some embodiments, an anti-PD-1 antibody provided herein binds to PD-1 with a K_{D} of 1-10 nM, 1-5 nM, 1-2 nM, 0.5-1 nM, 0.1-0.5 nM, 0.05-0.1 nM, 0.01-0.05 nM, or 0.005-0.01 nM. In some embodiments, an anti-PD-1 antibody or antigen-binding fragment thereof provided herein inhibits the interaction between PD-1 and PD-L1. The half-maximal inhibitory concentration (IC₅₀) for the interaction between PD-1 and PD-L1 may be determined using a suitable method known in the art or described herein, including, for example, commercially available kits. In some embodiments, an anti-PD-1 antibody or antigen-binding fragment thereof provided herein inhibits the interaction between PD-1 and PD-L1 with an IC₅₀ of less than about 500 ng/mL, less than about 400 ng/mL, or less than about 300 ng/mL.

In some embodiments, an anti-PD-1 antibody or antigen-binding fragment thereof induces activation of T cells. The activation of T cells can be determined using any suitable method described herein or known in the art, for example, a mixed lymphocyte reaction (MLR) assay. In such assays, antibodies are added to antigen presenting cells and effector cells, and the levels of cytokines such as interleukin 2 (IL-2) and/or interferons such as interferon gamma (IFN-γ) may be quantified using ELISA or commercially available kits. In some embodiments, an anti-PD-1 antibody or antigen-binding fragment thereof described herein increases the production of IL-2 by a population of T cells by about 10-25%, about 25-50%, about 50-75%, about 75-100%, about 2-3-fold, about 3-4-fold, about 4-5-fold, about 5-10-fold, about 10-15-fold, or about 15-20-fold. In some embodiments, an anti-PD-1 antibody or antigen-binding fragment thereof described herein increases the production of IFN-γ by a population of T cells by about 10-25%, about 25-50%, about 50-75%, about 75-100%, about 2-3-fold, about 3-4-fold, about 4-5-fold, about 5-10-fold, about 10-15-fold, or about 15-20-fold.

It can be desirable to modify an antibody disclosed herein with respect to effector function, so as to enhance, e.g., the effectiveness of the antibody in treating diseases and disorders associated. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). Alternatively, an antibody can be engineered that has dual Fc regions and can thereby have enhanced complement lysis and ADCC capabilities. Mutations affecting the effector function of antibodies are known in the art, see. e.g., Saunders et al., 2019; Front. Immunol. 10:1296 and Wang et al., 2018. Protein Cell 9(1): 63-73, each of which is incorporated herein by reference in its entirety for examples of Fc modifications that may be introduced in the antibodies described herein. Unless otherwise indicated, the Fc mutations in this section are described with reference to the Kabat numbering scheme for immunoglobulins.

Such antibodies may exhibit increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization to the brain, kidney, and/or liver is desired. In one exemplary embodiment, the altered antibodies of the invention exhibit reduced transport across the epithelium of kidney glomeruli from the vasculature. In another embodiment the altered antibodies of the invention exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering).

Illustrative mutations and combinations of mutations that may be introduced into the Fc of an anti-FcRn antibody provided herein include, without limitation, Lys326Trp/Glu333Ser, Ser267Glu/His268Phe/Ser324Thr, Lys326Trp/Glu333Ser, Lys326Ala/Glu333Ala. Lys326Met/Glu333Ser, Cys221Asp/Asp222Cys, Ser267Glu, His268Phe, Ser324Thr, Glu345Arg, S239D/1332E, S239D/1332E/A330L, Arg435His, Met252Tyr/Ser254Thr/Thr256Glu (“YTE”), Met428Leu/Asn434Ser, Thr252Leu/Thr253Ser/Thr254Phe, Leu235Glu, Leu234Ala/Leu235Ala (“LALA”). Ser228Pro/Leu235Glu, Leu234Ala/Leu235Ala/Pro329Gly, Pro331Ser/Leu234Glu/Leu235Phe, Asp265Ala, and Ala330Leu.

The present disclosure includes all antibodies and antibody fragments that bind to the same antigen or epitope as the antibodies or antibody fragments disclosed herein. A person skilled in the art will appreciate that binding assays such as a competition binding assay can be used to find other antibodies and antibody fragments with the same binding specificities as the antibodies and antibody fragments disclosed herein.

Those skilled in the art will recognize that it is possible to determine, without undue experimentation, if an antibody (e.g., monoclonal antibody) has the same specificity as an antibody or antibody fragment disclosed herein by ascertaining whether the former prevents the latter from binding to PD-1. If the monoclonal antibody being tested competes with the monoclonal antibody disclosed herein, as shown by a decrease in binding by the monoclonal antibody disclosed herein, then the two monoclonal antibodies bind to the same, or a closely related, epitope.

Methods for the screening of antibodies that possess the desired specificity include, but are not limited to, enzyme linked immunosorbent assay (ELISA) and other immunologically mediated techniques known within the art.

An illustrative method for determining whether a monoclonal antibody has the specificity of an antibody or antibody fragment disclosed herein is to pre-incubate the monoclonal antibody disclosed herein with a soluble PD-1, and then add the monoclonal antibody being tested to determine if the monoclonal antibody being tested is inhibited in its ability to bin PD-1. If the monoclonal antibody being tested is inhibited then, in all likelihood, it has the same, or functionally equivalent, epitopic specificity as the monoclonal antibody disclosed herein.

The disclosure also includes an immunoconjugate comprising (1) a binding protein disclosed herein, preferably an antibody or antibody fragment, that has been attached to (2) an effector molecule.

Heteroconjugate antibodies are also within the scope of the present invention. Heteroconjugate antibodies are composed of two covalently joined antibodies.

In another aspect, provided herein is a conjugate comprising and anti-PD-1 antibody or antigen-binding fragment thereof provided herein and an effector molecule. In one embodiment, the effector molecule is a label, which can generate a detectable signal, directly or indirect. Examples of labels include radioactive isotopes (i.e., a radioconjugate). In another embodiment, the effector molecule is a therapeutic agent. Therapeutic agents include, but are not limited to, antineoplastic agents. In yet another embodiment, the therapeutic agent is a toxin.

The term “cancer therapeutic agent” or “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

A toxin is an enzymatically active toxin of bacterial, fungal, plant, or animal origin, or fragments thereof). Toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

### Polynucleotides and Vectors

In another aspect, the present disclosure is directed to polynucleotide sequences that encode the anti-PD-1 antibodies and antigen-binding fragments thereof described herein. Exemplary polynucleotide sequences encoding the CDRs and the heavy and light chains of the anti-PD-1 antibodies described herein are set forth in Table 2.

In some embodiments, provided herein is a polynucleotide encoding a VH comprising the sequence set forth in any one of SEQ ID Nos: 80, 88, 96, 104, 112, 120, 128, 136, 137, 138, 139, 140, 144, 145, 146, 147, 148, 149, 153, 154, 155, and 156.

In some embodiments, a polynucleotide encoding the VH of an anti-PD-1 antibody or antigen-binding fragment thereof provided herein comprises a nucleic acid sequence that is at least 80%, at least 85%, at least 90%, at least 95%, at least 96%, at least 97%, at least 98%, or at least 99% identical to the sequence set forth in any one of SEQ ID NOs: 1, 9, 17, 25, 33, 41, 49, 57, 58, 59, 60, 61, 65, 66, 67, 68, 69, 70, 74, 75, 76, and 77. In some embodiments, a polynucleotide encoding the VH of an anti-PD-1 antibody or antigen-binding fragment thereof provided herein comprises the nucleic acid sequence set forth in any one of SEQ ID NOs: 1, 9, 17, 25, 33, 41, 49, 57, 58, 59, 60, 61, 65, 66, 67, 68, 69, 70, 74, 75, 76, and 77.

In some embodiments, provided herein is a polynucleotide encoding a VL comprising the sequence set forth in any one of SEQ ID NOs: 84, 92, 100, 108, 116, 124, 132, 141, 142, 143, 150, 151, 152, 157, and 158.

In some embodiments, a polynucleotide encoding the VL of an anti-PD-1 antibody or antigen-binding fragment thereof provided herein comprises a nucleic acid sequence that is at least 80%, at least 85%, at least 90%, at least 95%, at least 96%, at least 97%, at least 98%, or at least 99% identical to the sequence set forth in any one of SEQ ID NOs: 5, 13, 21, 29, 37, 45, 53, 62, 63, 64, 71, 72, 73, 78, and 79. In some embodiments, a polynucleotide encoding the VL of an anti-PD-1 antibody or antigen-binding fragment thereof provided herein comprises the nucleic acid sequence set forth in any one of SEQ ID NOs: 5, 13, 21, 29, 37, 45, 53, 62, 63, 64, 71, 72, 73, 78, and 79.

TABLE 2
Nucleotide sequences of anti-PD-1 antibodies

| SEQ ID | | |
|---|---|---|
| NO | Description | Sequence |
| 1 | CUSB0103- | ATGAACTTCGGGCTCAGATTGATTTTCCTTGTCCTTACTTTA |
| | VH | AAAGGTGTCCAGTGTGACGTGAAGCTGGTGGAGTCTGGGGG |
| | | AGGCTTGGTGAAGCCTGGAGGGTCCCTGAAACTCTCCTGTG |
| | | CAGCCTCTGGATTCACTTTCAGTAGCTATACCATGTCTTGGG |
| | | TTCGCCAGACTCCGGAGAAGAGGCTGGAGTGGGTCGCAGCC |
| | | ATTAGTGGTGGTGGTAGTTACACCTACTATGCAGACAGTGT |
| | | TAAGGGCCGATTCACCATCTCCAGAGACAATGCCAAGAACA |
| | | CCCTGTCCCTGCAAATGAGCAGTCTGAAGTCTGAGGACACA |
| | | GCCATGTATTTCTGTACAAGTCCCTATGATTTCGCTATGGAC |
| | | TACTGGGGTCAAGGAACCTCAGTCACCGTCTCTTCA |
| 2 | CUSB0103- | AGCTATACCATGTCT |
| | VH-CDR1 | |
| 3 | CUSB0103- | GCCATTAGTGGTGGTGGTAGTTACACCTACTATGCAGACAG |
| | VH-CDR2 | TGTTAAGGGC |
| 4 | CUSB0103- | CCCTATGATTTCGCTATGGACTAC |
| | VH-CDR3 | |
| 5 | CUSB0103- | ATGTCCTCTGCTCAGTTCCTTGGTCTCCTGTTGCTCTGTTTTC |
| | VL | AAGGTACCAGATGTGATATCCAGATGACACAGACTACATCC |
| | | TCCCTGTCTGCCTCTCTGGGAGACAGAGTCACCATCAGTTGC |
| | | AGGGCAAGTCAGGACATTACCAATTATTTAAACTGGTATCA |
| | | ACAGAAACCAGATGGATCTGTTAAACTCCTGATCTACTACA |
| | | CATCAAGATTACACTCAGGAGTCCCATCAAGGTTCCGTGGC |
| | | AGTGGGTCTGGAACAGATTATTCTCTCACCATTAGTAACCT |
| | | GGAGCAAGAAGATATTGCCACTTACTTTTGCCAACAGGGTA |
| | | ATACGCTTCCGTGGACGTTCGGTGGAGGCACCAAGCTGGAA |
| | | ATCAAA |
| 6 | CUSB0103- | AGGGCAAGTCAGGACATTACCAATTATTTAAAC |
| | VL-CDR1 | |
| 7 | CUSB0103- | TACACATCAAGATTACACTCA |
| | VL-CDR2 | |
| 8 | CUSB0103- | CAACAGGGTAATACGCTTCCGTGGACG |
| | VL-CDR3 | |
| 9 | CUSB0123- | ATGGGTTGGAGCTGTATCATCTTCTTTCTGGTAGCAACAGCT |
| | VH | ACAGGTGTGCACTCCCAGGTCCAGCTACAACAGTCTGGGCC |
| | | TGAGCTGGTGAGGCCTGGGGAATCAGTGAAGATTTCCTGTA |
| | | AGGGTTCCGGCTACACATTCACTGATTATGTTATGCACTGG |
| | | GTGAAGCAGAGTCATGCAAAGAGTCTAGAGTGGATTGGAG |
| | | TTATTACTATTTACAATGATATTACAAACTACAACCAGAAA |
| | | TTTAAGGGCAAGGCCACAATGACTATAGACAAATCCTCCAG |
| | | CACAGCCTATATGGAACTTGCCAGATTGACATCTGAGGATT |
| | | CTGCCATCTATTACTGTGCAAGAGAGGGGTATGATTCCTAC |
| | | TGGTACTTCGATGTCTGGGGCGCAGGGACCACGGTCACCGT |
| | | CTCCTCA |
| 10 | CUSB0123- | GATTATGTTATGCAC |
| | VH-CDR1 | |
| 11 | CUSB0123- | GTTATTACTATTTACAATGATATTACAAACTACAACCAGAA |
| | VH-CDR2 | ATTTAAGGGC |
| 12 | CUSB0123- | GAGGGGTATGATTCCTACTGGTACTTCGATGTC |
| | VH-CDR3 | |
| 13 | CUSB0123- | ATGAAGTTGCCTGTTAGGCTGTTGGTGCTGATGTTCTGGATT |
| | VL | CCTGCTTCCAGCAGTGATGTTTTGATGACCCAAACTCCACTC |
| | | TCCCTGCCTGTCAGTCTTGGAGATCAAGCCTCCATCTCTTGC |
| | | AGATCTAGTCAAACCATTGTACATAGTAATGGAAACACCTA |
| | | TTTAGAATGGTACCTGCAGAAACCAGGCCAGTCTCCAAAGC |
| | | TCCTGATCTACAAAGTTTCCAACCGATTTTCTGGGGTCCCAG |
| | | ACAGGTTCAGTGGCAGTGGATCAGGGACAGATTTTACACTC |
| | | AAGATCAGCAGAGTGGAGGCTGAGGATCTGGGAGTTTATTA |
| | | CTGCTTTCAAGGTTCACATCTTCCGTACACGTTCGGAGGGG |
| | | GGACCAAGCTGGAAATAAAA |
| 14 | CUSB0123- | AGATCTAGTCAAACCATTGTACATAGTAATGGAAACACCTA |
| | VL-CDR1 | TTTAGAA |
| 15 | CUSB0123- | AAAGTTTCCAACCGATTTTCT |
| | VL-CDR2 | |
| 16 | CUSB0123- | TTTCAAGGTTCACATCTTCCGTACACG |
| | VL-CDR3 | |
| 17 | CUSB0124- | ATGGATTGGCTGTGGAACTTGCTATTCCTGATGGCAGCTGC |
| | VH | CCAAAGTATCCAAGCACAGATCCAGTTGGTGCAGTCTGGAC |
| | | CTGAGCTGAAGAAGCCTGGAGAGACAGTCAAGATCTCCTGC |
| | | AAGGCTTCTGGGTATATCTTCACAAACTATGGAGTGACCTG |
| | | GGTGAAGCAGACTCCAGGAAAGGGTTTAAAGTGGATGACC |
| | | TGGATAAACACCAACACTGGAGAGCCAACATCTGCTGAAG |
| | | AGTTCAAGGGACGGTTTGACTTCTCTTCGGAAACCTCTGCC |
| | | ACCACTGCCTATTTGCAGATCAACAACCTCAAAAATGAGGA |
| | | CACGGCTACATATTTCTGTGTAAGAATACGGGAGAATGCTA |
| | | TGGACTACTGGGGTCAAGGAACCTCAGTCACCGTCTCCTCA |
| 18 | CUSB0124- | AACTATGGAGTGACC |
| | VH-CDR1 | |
| 19 | CUSB0124- | TGGATAAACACCAACACTGGAGAGCCAACATCTGCTGAAG |
| | VH-CDR2 | AGTTCAAGGGA |
| 20 | CUSB0124- | ATACGGGAGAATGCTATGGACTAC |
| | VH-CDR3 | |
| 21 | CUSB0124- | ATGAACATGCTCACTCAGCTCCTGGGATTACTGCTGCTCTGG |
| | VL | TTTGCAGGTGGTAAATGTGACATTCAGATGACCCAGTCTCC |
| | | TGCCTCCCAGTCTGCATCTCTGGGAGAAAGTGTCACCATCA |
| | | CATGCCTGGCAAGTCAGACCATTGGTACCTGGTTGGCATGG |
| | | TATCAGCAGAAACCAGGGAAATCTCCTCAGCTCCTGATTTA |
| | | TGCTGCAACCAGGTTGGCAGTTGGGGTCCCATCAAGGTTCA |
| | | GTGGTAGTGGATCTGGCACAAAATTTTCTTTCAAGATCAGC |
| | | AGACTGCAGGCTGAAGATTTTGTAAGTTATTACTGTCAACA |
| | | ACTTTACAGTACTCCATTCACGTTCGGCTCGGGGACAAAGT |
| | | TGGAGATAAAA |
| 22 | CUSB0124- | CTGGCAAGTCAGACCATTGGTACCTGGTTGGCA |
| | VL-CDR1 | |
| 23 | CUSB0124- | GCTGCAACCAGGTTGGCAGTT |
| | VL-CDR2 | |
| 24 | CUSB0124- | CAACAACTTTACAGTACTCCATTCACG |
| | VL-CDR3 | |
| 25 | CUSB0125- | ATGAGAGTGCTGATTCTTTTGTGGCTGTTCACAGCCTTTCCT |
| | VH | GGTATCCTGTCTGATGTGCAGCTTCGGGAGTCGGGACCTGG |
| | | CCTGGTGAAACCTTCTCAGTCTCTGTCCCTCACCTGCACTGT |
| | | CACTGGCTACTCAATCACCAGTGATTATGCCTGGAACTGGA |
| | | TCCGGCAGTTTCCAGGAAACAAACTGGAGTGGATGGGCTAC |
| | | ATATACTACAGTGGTACCACTAGTTACAACCCATCTCTCAA |
| | | AAGTCGAATCTCTATCACTCGAGACACATCCAAGAACCAGT |
| | | TCTTCCTGCAGTTGAATTCTGTGACTACTGAGGACACAGCC |
| | | ACGTATTACTGTGCAAGAAACTACGGTAGTGCCTTTTACTA |
| | | CTTTGACTACTGGGGCCAAGGCACCACTCTCACAGTCTCCTC |
| | | A |
| 26 | CUSB0125- | AGTGATTATGCCTGGAAC |
| | VH-CDR1 | |
| 27 | CUSB0125- | TACATATACTACAGTGGTACCACTAGTTACAACCCATCTCTC |
| | VH-CDR2 | AAAAGT |
| 28 | CUSB0125- | AACTACGGTAGTGCCTTTTACTACTTTGACTAC |
| | VH-CDR3 | |
| 29 | CUSB0125- | ATGGTCCTTGCTCAGTTTCTTGCATTCTTGTTGCTTTGGTTTC |
| | VL | CAGGTGCAAGATGTGACATCCTGATGACCCAATCTCCATCC |
| | | TCCATGTCTGTTTCTCTGGGAGACACAGTCACCATCACTTGC |
| | | CATGCAAGTCAGGGCATTAGTAGTAATATAGGGTGGTTGCA |
| | | GCAGAAACCAGGGAAATCATTTAAGGGCCTGATCTATCGTG |
| | | GAACCAACTTGGAAGATGGAGTTCCATCAAGGTTCAGTGGC |
| | | AGTGGATCTGGAGCAGATTATTCTCTCACCATCAGCAGCCT |
| | | GGAATCTGAGGATTTTGCGGACTATTACTGTGTACAGTATG |
| | | CTCAGTTTCCTCCGACGTTCGGTGGAGGCACCAAGCTGGAA |
| | | ATCAAA |
| 30 | CUSB0125- | CATGCAAGTCAGGGCATTAGTAGTAATATAGGG |
| | VL-CDR1 | |
| 31 | CUSB0125- | CGTGGAACCAACTTGGAAGAT |
| | VL-CDR2 | |
| 32 | CUSB0125- | GTACAGTATGCTCAGTTTCCTCCGACG |
| | VL-CDR3 | |
| 33 | CUSB0126- | ATGAACTTTGGGCTGAGCTTGATTTTCCTTGTCCTAATTTTA |
| | VH | AAAGGTGTCCAATGTGAGGTGAAGCTGGTGGAGTCTGGGG |
| | | GAGACTTAGTGAAGCCTGGAGGGTCCCTGAAACTCTCCTGT |
| | | GCAGCCTCTGGATTCCCTTTCAGTAGCTATCTCATGTCTTGG |
| | | GTTCGCCAGACTCCGGAGAAGAGGCTGGAGTGGGTCGCAG |
| | | CCATTAGTGGTGGTGGTAGAGACACCTACTATCCAGACAAT |
| | | GTGAAGGGTCGATTCACCATCTCCAGAGACAATGCCAGGAA |
| | | CATCCTGTATCTGCAAATGAGCAGTCTGAGGTCTGAGGACA |
| | | CGGCCGTGTATTACTGTGCAACAAGGGGCTCCTATGCTATG |
| | | GACTATTGGGGGCAAGGAACCTCAGTCACCGTCTCCTCA |
| 34 | CUSB0126- | AGCTATCTCATGTCT |
| | VH-CDR1 | |
| 35 | CUSB0126- | GCCATTAGTGGTGGTGGTAGAGACACCTACTATCCAGACAA |
| | VH-CDR2 | TGTGAAGGGT |
| 36 | CUSB0126- | AGGGGCTCCTATGCTATGGACTAT |
| | VH-CDR3 | |
| 37 | CUSB0126- | ATGCTCACTCAGCTCCTGGGATTTCTGCTGCTCTGGTTTGCA |
| | VL | GGTGGTAAATGTGACATTCAGATGACCCAGTCTCCTGCCTC |
| | | CCAGTCTGCATCTCTGGGAGAAAGTGTCACCATCACATGCC |
| | | TGGCAAGTCAGACCATTGGTACATGGTTAGCATGGTATCAG |
| | | CAGAAACCAGGGAAATCTCCTCAGATCCTGATTTATGCTGC |
| | | AACCAACTTGGCAGATGGGGTCCCATCAAGGTTCAGTGGTA |
| | | GTGGATCTGGCACAAAATTTTCTTTCAAGATCAGCAGCCTA |
| | | CAGGCTGAAGATTTTGTAAGTTATTACTGTCAACAACTTTAC |
| | | AGTATTCCGTGGACGTTCGGTGGAGGCACCAAGCTGGAAAT |
| | | CAAA |
| 38 | CUSB0126- | CTGGCAAGTCAGACCATTGGTACATGGTTAGCA |
| | VL-CDR1 | |
| 39 | CUSB0126- | GCTGCAACCAACTTGGCAGAT |
| | VL-CDR2 | |
| 40 | CUSB0126- | CAACAACTTTACAGTATTCCGTGGACG |
| | VL-CDR3 | |
| 41 | CUSB0129- | ATGAACTTTGGGCTGAGCTTGATTTTCCTTGTCCTAATTTTA |
| | VH | AAAGGTGTCCAATGTGAGGTGAAGCTGGTGGAGTCTGGGG |
| | | GAGGCTTAGTGAAGCCTGGAGGGTCCCTGAAACTCTCCTGT |
| | | GCAGCCTCTGGATTCCCTTTCAGTAGCTATCTCATGTCTTGG |
| | | GTTCGCCAGACTCCGGAGAAGAGGCTGGAGTGGGTCGCAG |
| | | CCATTAGTGGTGGTGGTAGAGACACCTACTATCCAGACAGT |
| | | GTGAAGGGTCGATTCACCATCTCCAGAGACAATGCCAGGAA |
| | | CATCCTGTACCTGCAAATGAGCAGTCTGAGGTCTGAGGACA |
| | | CGGCCTTGTATTACTGTGCAAGAAGGGGCTCCTATGCTATG |
| | | GACTACTGGGGGCAAGGAACCTCAGTCACCGTCTCCTCA |
| 42 | CUSB0129- | AGCTATCTCATGTCT |
| | VH-CDR1 | |
| 43 | CUSB0129- | GCCATTAGTGGTGGTGGTAGAGACACCTACTATCCAGACAG |
| | VH-CDR2 | TGTGAAGGGT |
| 44 | CUSB0129- | AGGGGCTCCTATGCTATGGACTAC |
| | VH-CDR3 | |
| 45 | CUSB0129- | ATGCTCACTCAGCTCCTGGGATTACTGCTGCTCTGGTTTGCA |
| | VL | GGTGGTAAATGTGACATTCAGATGACCCAGTCTCCTGCCTC |
| | | CCAGTCTGCATCTCTGGGAGAAAGTGTCACCATCACATGCC |
| | | TGGCAAGTCAGACCATTGGTACATGGTTAGCATGGTATCAG |
| | | CAGAAACCAGGGAAATCTCCTCAGATCCTGATTTATGATGC |
| | | AACCAACTTGGCAGATGGGGTCCCATCAAGGTTCAGTGGTA |
| | | GTGGATCTGGCACAAAATTTTCTTTCAAGATCAGCAGCCTA |
| | | CAGGCTGAAGATTTTGTTAGTTATTACTGTCAACAACTTTAC |
| | | AGTATTCCGTGGACGTTCGGTGGAGGCACCAAGCTGGAAAT |
| | | CAAA |
| 46 | CUSB0129- | CTGGCAAGTCAGACCATTGGTACATGGTTAGCA |
| | VL-CDR1 | |
| 47 | CUSB0129- | GATGCAACCAACTTGGCAGAT |
| | VL-CDR2 | |
| 48 | CUSB0129- | CAACAACTTTACAGTATTCCGTGGACG |
| | VL-CDR3 | |
| 49 | CUSB0136- | ATGGCTGTCCTGGCACTACTCCTCTGCCTGGTGGCTTTCCCA |
| | VH | AGCTGTACCCTGTCCCAGGTGCAACTGAAGGAGTCAGGACC |
| | | TGGCCTGGTGGCGCCCTCACAGAGCCTGTCCATTACCTGCA |
| | | CTGTCTCTGGGTTCTCATTAACCACCTATGATATAATCTGGA |
| | | TTCGCCAGTCACCAGGAAAGGGTCTGGAGTGGCTTGGAGTA |
| | | ATATGGACTGGTGGAGACACAAATTATAATTCAGCTTTCAT |
| | | GTCCAGACTGAGCATCAGCAAGGACAACTCCAAGAGCCAA |
| | | ATTTTCTTAAAAATGAACAGTCTGCAAACTGATGACACAGC |
| | | CATATATTACTGTGTAAGAGATTGGGCTTACTGGGGCCAAG |
| | | GGACTCTGGTCACTGTCTCTACA |
| 50 | CUSB0136- | ACCTATGATATAATC |
| | VH-CDR1 | |
| 51 | CUSB0136- | GTAATATGGACTGGTGGAGACACAAATTATAATTCAGCTTT |
| | VH-CDR2 | CATGTCC |
| 52 | CUSB0136- | GATTGGGCTTAC |
| | VH-CDR3 | |
| 53 | CUSB0136- | ATGGAGACAGACACACTCCTGCTATGGGTGCTGCTGCTCTG |
| | VL | GGTCCCAGGTTCCACAGGTAACATTGTGCTGACCCAATCTC |
| | | CAGCTTCTTTGGCTGTGTCTCTAGGGCAGAGGGCCACCATA |
| | | TCCTGCAGAGCCAGTGAAAGTGTTGATAATTATGGCAATAG |
| | | TTTTATACAATGGTACCAGCAGAAACCAGGACAGCCACCCA |
| | | AACTCCTCATCTATTTTGCATCCAACCTACAATCTGGGGTCC |
| | | CTGCCAGGTTCAGTGGCAGTGGGTCTAGGACAGACTTCACC |
| | | CTCACCATTGATCCTGTGGAGGCTGATGATGCTGCAACCTA |
| | | TTACTGTCATCAAAATAATGAGGATCCATTCACGTTCGGCTC |
| | | GGGGACAAAGTTGGAAATAAAA |
| 54 | CUSB0136- | AGAGCCAGTGAAAGTGTTGATAATTATGGCAATAGTTTTAT |
| | VL-CDR1 | ACAA |
| 55 | CUSB0136- | TTTGCATCCAACCTACAATCT |
| | VL-CDR2 | |
| 56 | CUSB0136- | CATCAAAATAATGAGGATCCATTCACG |
| | VL-CDR3 | |
| 57 | CUSB0125- | CAAGTGCAGCTGCAGGAGAGCGGCCCTGGACTGGTCAAGC |
| | BMVH.1 | CTTCTCAGACCCTGTCTCTCACCTGTACCGTGTCCGGCTACA |
| | | GCATCACCAGCGACTACGCCTGGAATTGGATCAGACAGCCT |
| | | CCAGGCAAGGGCCTGGAATGGATCGGCTATATCTACTATTC |
| | | TGGAACAACATCCTACAACCCCAGCCTGAAAAGCAGAGTG |
| | | ACAATCAGCGTGGACACCTCCAAGAACCAGTTCAGCCTGAA |
| | | GCTGAGCAGCGTGACCGCCGCTGATACAGCCGTGTACTACT |
| | | GCGCCAGAAACTACGGCAGCGCCTTTTACTACTTCGACTAC |
| | | TGGGGCCAGGGCACAACCCTGACCGTGTCTAGC |
| 58 | CUSB0125- | CAAGTGCAGCTGCAGGAGAGCGGCCCTGGACTGGTCAAGC |
| | BMVH.2 | CTTCTCAGACCCTGTCTCTCACCTGTACCGTGTCCGGCTACA |
| | | GCATCACCAGCGACTACGCCTGGAATTGGATCAGACAGCCT |
| | | CCAGGCAAGGGCCTGGAATGGATGGGCTATATCTACTATTC |
| | | TGGAACAACATCCTACAACCCCAGCCTGAAAAGCAGAATCA |
| | | CAATCAGCCGGGACACCTCCAAGAACCAGTTCAGCCTGAAG |
| | | CTGAGCAGCGTGACCGCCGCTGATACAGCCACCTACTACTG |
| | | CGCCAGAAACTACGGCAGCGCCTTTTACTACTTCGACTACT |
| | | GGGGCCAGGGCACAACCCTGACCGTGTCTAGC |
| 59 | CUSB0125- | CAAGTGCAGCTGCAGGAGAGCGGCCCTGGACTGGTCAAGC |
| | BMVH.3 | CTTCTCAGACCCTGTCTCTCACCTGTACCGTGTCCGGCTACA |
| | | GCATCACCAGCGACTACGCCTGGAATTGGATCAGACAGCCT |
| | | CCAGGCAAGGGCCTGGAATGGATGGGCTATATCTACTATTC |
| | | TGGAACAACATCCTACAACCCCAGCCTGAAAAGCAGAATCT |
| | | CCATCACCCGGGACACCTCCAAGAACCAGTTCAGCCTGAAG |
| | | CTGAGCAGCGTGACCGCCGCTGATACAGCCGTGTACTACTG |
| | | CGCCAGAAACTACGGCAGCGCCTTTTACTACTTCGACTACT |
| | | GGGGCCAGGGCACAACCCTGACCGTGTCTAGC |
| 60 | CUSB0125- | CAAGTGCAGCTGCAGGAGAGCGGACCTGGCCTGGTCAAGC |
| | BMVH.4 | CTAGCCAGACCCTGAGCCTGACCTGTACCGTGTCTGGATAT |
| | | AGCATCACATCTGACTACGCCTGGAATTGGATCAGACAGTT |
| | | TCCAGGCAAGAAGCTGGAATGGATGGGCTACATCTACTACA |
| | | GCGGCACCACCTCCTACAACCCCAGCCTGAAAAGCAGAATC |
| | | AGCATCACCCGGGACACCAGCAAGAACCAGTTCAGCCTCAA |
| | | GCTGTCTTCTGTGACCGCCGCTGATACAGCCGTGTACTACTG |
| | | CGCCAGAAACTACGGCAGCGCCTTCTACTATTTCGACTACT |
| | | GGGGCCAGGGCACAACACTGACAGTGTCCTCC |
| 61 | CUSB0125- | CAAGTGCAGCTGCAGGAGAGCGGACCTGGCCTGGTCAAGC |
| | BMVH.5 | CTAGCCAGACCCTGAGCCTGACCTGTACCGTGTCTGGATAT |
| | | AGCATCACATCTGACTACGCCTGGAATTGGATCAGACAGTT |
| | | TCCAGGCAAGAAGCTGGAATGGATGGGCTACATCTACTACA |
| | | GCGGCACCACCTCCTACAACCCCAGCCTGAAAAGCAGAATC |
| | | AGCATCACCCGGGACACCAGCAAGAACCAGTTCAGCCTCCA |
| | | GCTGTCTTCTGTGACCGCCGCTGATACAGCCACCTACTACTG |
| | | CGCCAGAAACTACGGCAGCGCCTTCTACTATTTCGACTACT |
| | | GGGGCCAGGGCACAACACTGACAGTGTCCTCC |
| 62 | CUSB0125- | GATATCCAGATGACCCAGAGCCCTAGCAGCGTGTCCGCCAG |
| | BMVL.1 | CGTCGGCGACAGAGTGACCATTACATGTCACGCTTCTCAGG |
| | | GCATCAGCAGCAACATCGGCTGGTACCAGCAGAAACCTGGC |
| | | AAGGCCCCCAAGCTGCTGATCTACCGGGGCACAAACCTGGA |
| | | GGACGGCGTGCCATCTAGATTCAGCGGATCTGGCAGCGGAA |
| | | CAGACTTCACCCTGACCATCTCCAGCCTGCAACCTGAGGAT |
| | | TTCGCCACCTACTACTGCGTGCAGTATGCCCAGTTCCCCCCC |
| | | ACCTTCGGCGGAGGCACCAAGCTGGAAATCAAG |
| 63 | CUSB0125- | GATATCCAGATGACCCAGAGCCCTAGCAGCGTGTCCGCCAG |
| | BMVL.2 | CGTCGGCGACAGAGTGACCATTACATGTCACGCTTCTCAGG |
| | | GCATCAGCAGCAACATCGGCTGGCTGCAGCAGAAACCTGGC |
| | | AAGGCCCCCAAGCTGCTGATCTACCGGGGCACAAACCTGGA |
| | | GGACGGCGTGCCATCTAGATTCAGCGGATCTGGCAGCGGAA |
| | | CAGACTACACCCTGACCATCTCCAGCCTGCAACCTGAGGAT |
| | | TTCGCCACCTACTACTGCGTGCAGTATGCCCAGTTCCCCCCC |
| | | ACCTTCGGCGGAGGCACCAAGCTGGAAATCAAG |
| 64 | CUSB0125- | GATATCCAGATGACCCAGAGCCCTAGCAGCGTGTCCGCCAG |
| | BMVL.3 | CGTCGGCGACAGAGTGACCATTACATGTCACGCTTCTCAGG |
| | | GCATCAGCAGCAACATCGGCTGGCTGCAGCAGAAACCTGGC |
| | | AAGGCCTTTAAGGGCCTGATCTACCGGGGCACAAACCTGGA |
| | | GGACGGCGTGCCATCTAGATTCAGCGGATCTGGCAGCGGAA |
| | | CAGACTACACCCTGACCATCTCCAGCCTGCAACCTGAGGAT |
| | | TTCGCCACCTACTACTGCGTGCAGTATGCCCAGTTCCCCCCC |
| | | ACCTTCGGCGGAGGCACCAAGCTGGAAATCAAG |
| 65 | CUSB0136- | CAAGTGCAGCTGCAGGAGAGCGGACCTGGACTGGTCAAGC |
| | BMVH.1 | CCAGCGAGACACTGTCTCTGACATGTACCGTGTCTGGCTTC |
| | | AGCCTGACAACCTACGACATCATCTGGATCCGGCAGCCCCC |
| | | TGGCAAGGGCCTGGAATGGATCGGCGTGATCTGGACCGGCG |
| | | GCGACACCAATTACAACAGCGCCTTCATGTCTAGAGTGACC |
| | | ATCAGCGTGGACACCAGCAAAAACCAGTTCTCCCTCAAGCT |
| | | GAGCAGCGTGACCGCCGCTGATACAGCCGTGTACTACTGCG |
| | | CCAGAGATTGGGCCTATTGGGGCCAGGGCACCCTGGTGACC |
| | | GTGTCCTCC |
| 66 | CUSB0136- | CAAGTGCAGCTGCAGGAGAGCGGACCTGGACTGGTCAAGC |
| | BMVH.2 | CCAGCGAGACACTGTCTATTACATGTACCGTGTCTGGCTTCA |
| | | GCCTGACAACCTACGACATCATCTGGATCCGGCAGAGCCCT |
| | | GGCAAGGGCCTGGAATGGCTGGGCGTGATCTGGACCGGCG |
| | | GCGACACCAATTACAACAGCGCCTTCATGTCTAGACTGAGC |
| | | ATCAGCAAGGACACCAGCAAAAACCAGATCTCCCTCAAGAT |
| | | GAGCAGCCTGACCGCCGCTGATACAGCCGTGTACTACTGCG |
| | | CCAGAGATTGGGCCTATTGGGGCCAGGGCACCCTGGTGACC |
| | | GTGTCCTCC |
| 67 | CUSB0136- | CAAGTGCAGCTGCAGGAGAGCGGACCTGGACTGGTCAAGC |
| | BMVH.3 | CCAGCGAGACACTGTCTCTGACATGTACCGTGTCTGGCTTC |
| | | AGCCTGACAACCTACGACATCATCTGGATCCGGCAGAGCCC |
| | | TGGCAAGGGCCTGGAATGGCTGGGCGTGATCTGGACCGGCG |
| | | GCGACACCAATTACAACAGCGCCTTCATGTCTAGACTGACC |
| | | ATCAGCAAGGACACCAGCAAAAACCAGATCTCCCTCAAGAT |
| | | GAGCAGCCTGACCGCCGCTGATACAGCCGTGTACTACTGCG |
| | | CCAGAGATTGGGCCTATTGGGGCCAGGGCACCCTGGTGACC |
| | | GTGTCCTCC |
| 68 | CUSB0136- | CAAGTGCAGCTGCAGGAGAGCGGACCTGGACTGGTCAAGC |
| | BMVH.4 | CCAGCGAGACACTGTCTCTGACATGTACCGTGTCTGGCTTC |
| | | AGCCTGACAACCTACGACATCATCTGGATCCGGCAGAGCCC |
| | | TGGCAAGGGCCTGGAATGGCTGGGCGTGATCTGGACCGGCG |
| | | GCGACACCAATTACAACAGCGCCTTCATGTCTAGACTGAGC |
| | | ATCAGCAAGGACACCAGCAAAAACCAGTTCTCCCTCAAGAT |
| | | GAGCAGCCTGACCGCCGCTGATACAGCCGTGTACTACTGCG |
| | | CCAGAGATTGGGCCTATTGGGGCCAGGGCACCCTGGTGACC |
| | | GTGTCCTCC |
| 69 | CUSB0136- | CAAGTGCAGCTGCAGGAGAGCGGACCTGGACTGGTCAAGC |
| | BMVH.5 | CCAGCGAGACACTGTCTCTGACATGTACCGTGTCTGGCTTC |
| | | AGCCTGACAACCTACGACATCATCTGGATCCGGCAGAGCCC |
| | | TGGCAAGGGCCTGGAATGGCTGGGCGTGATCTGGACCGGCG |
| | | GCGACACCAATTACAACAGCGCCTTCATGTCTAGACTGACC |
| | | ATCAGCAAGGACACCAGCAAAAACCAGTTCTCCCTCAAGAT |
| | | GAGCAGCCTGACCGCCGCTGATACAGCCGTGTACTACTGCG |
| | | CCAGAGATTGGGCCTATTGGGGCCAGGGCACCCTGGTGACC |
| | | GTGTCCTCC |
| 70 | CUSB0136- | CAAGTGCAGCTGCAGGAGAGCGGACCTGGACTGGTCAAGC |
| | BMVH.6 | CCAGCGAGACACTGTCTCTGACATGTACCGTGTCTGGCTTC |
| | | AGCCTGACAACCTACGACATCATCTGGATCCGGCAGAGCCC |
| | | TGGCAAGGGCCTGGAATGGCTGGGCGTGATCTGGACCGGCG |
| | | GCGACACCAATTACAACAGCGCCTTCATGTCTAGACTGACC |
| | | ATCAGCAAGGACACCAGCAAAAACCAGTTCTCCCTCAAGAT |
| | | GAGCAGCGTGACCGCCGCTGATACAGCCGTGTACTACTGCG |
| | | CCAGAGATTGGGCCTATTGGGGCCAGGGCACCCTGGTGACC |
| | | GTGTCCTCC |
| 71 | CUSB0136- | GACATCGTGATGACACAGAGCCCCGACAGCCTGGCCGTGTC |
| | BMVL.1 | CCTGGGCGAGCGGGCCACAATTAACTGTAGAGCTTCTGAAA |
| | | GCGTCGACAACTACGGCAATAGCTTCATCCAGTGGTATCAG |
| | | CAGAAACCTGGCCAGCCTCCAAAGCTGCTGATCTACTTCGC |
| | | CTCCAACCTGCAAAGCGGCGTGCCTGATAGATTCAGCGGCT |
| | | CTGGCAGCGGAACAGACTTCACCCTGACCATCAGCAGCCTG |
| | | CAGGCCGAGGATGTGGCCGTGTACTACTGCCACCAGAACAA |
| | | CGAGGACCCCTTTACCTTCGGCGGCGGAACCAAGCTCGAAA |
| | | TCAAG |
| 72 | CUSB0136- | GACATCGTGCTGACACAGAGCCCCGACAGCCTGGCCGTGTC |
| | BMVL.2 | CCTGGGCGAGCGGGCCACAATTTCTTGTAGAGCTTCTGAAA |
| | | GCGTCGACAACTACGGCAATAGCTTCATCCAGTGGTATCAG |
| | | CAGAAACCTGGCCAGCCTCCAAAGCTGCTGATCTACTTCGC |
| | | CTCCAACCTGCAAAGCGGCGTGCCTGATAGATTCAGCGGCT |
| | | CTGGCAGCGGAACAGACTTCACCCTGACCATCAGCAGCGTG |
| | | CAGGCCGAGGATGTGGCCACCTACTACTGCCACCAGAACAA |
| | | CGAGGACCCCTTTACCTTCGGCGGCGGAACCAAGCTCGAAA |
| | | TCAAG |
| 73 | CUSB0136- | GACATCGTGCTGACACAGAGCCCCGACAGCCTGGCCGTGTC |
| | BMVL.3 | CCTGGGCGAGCGGGCCACAATTTCTTGTAGAGCTTCTGAAA |
| | | GCGTCGACAACTACGGCAATAGCTTCATCCAGTGGTATCAG |
| | | CAGAAACCTGGCCAGCCTCCAAAGCTGCTGATCTACTTCGC |
| | | CTCCAACCTGCAAAGCGGCGTGCCTGATAGATTCAGCGGCT |
| | | CTGGCAGCGGAACAGACTTCACCCTGACCATCAGCAGCCTG |
| | | CAGGCCGAGGATGTGGCCACCTACTACTGCCACCAGAACAA |
| | | CGAGGACCCCTTTACCTTCGGCGGCGGAACCAAGCTCGAAA |
| | | TCAAG |
| 74 | CUSB0136- | CAAGTGCAGCTGCAGGAGAGCGGCCCTGGCCTGGTCAAGCC |
| | BMVH.3a | TTCTGAAACCCTGAGCCTGACCTGTACAGTGTCTGGATTCA |
| | | GCCTGACAACCTACGACATCATTTGGATCCGGCAGAGCCCC |
| | | GGAAAAGGCCTCGAGTGGCTGGGCGTGATCTGGACCGGCG |
| | | GCGACACCTGGTACAACAGCGCCTTCATGTCTAGACTGACC |
| | | ATCTCCAAGGACACAAGCAAGAACCAGATCAGCCTGAAGA |
| | | TGAGCAGCCTGACAGCCGCTGATACCGCCGTGTACTACTGC |
| | | GCCAGAGATTGGGCCTATTGGGGCCAGGGCACACTGGTGAC |
| | | CGTGTCCAGC |
| 75 | CUSB0136- | CAAGTGCAGCTGCAGGAGTCCGGACCTGGCCTGGTCAAGCC |
| | BMVH.3b | TAGCGAGACACTGAGCCTCACCTGTACCGTGTCCGGCTTCA |
| | | GCCTGACAGTGTACGACATCATCTGGATCCGGCAGAGCCCC |
| | | GGAAAGGGCCTGGAATGGCTGGGCGTGATCTGGACCGGCG |
| | | GCGACACAACATACAACAGCGCCTTCATGTCTAGACTGACC |
| | | ATCAGCAAGGACACCAGCAAAAACCAGATTTCTCTGAAGAT |
| | | GAGCAGCCTGACAGCCGCTGATACCGCCGTGTATTACTGCG |
| | | CCAGAGATTGGATCTACTGGGGCCAGGGCACCCTGGTGACC |
| | | GTGTCTAGC |
| 76 | CUSB0136- | CAAGTGCAGCTGCAGGAGAGCGGACCTGGACTGGTCAAGC |
| | BMVH.3c | CCAGCGAGACACTGAGCCTGACCTGTACCGTGTCTGGCTTC |
| | | AGCCTGACAGTGTACGACATCATCTGGATCCGGCAGAGCCC |
| | | TGGCAAGGGCCTGGAATGGCTGGGCGTGATCTGGACCGGCG |
| | | GCGACACCACATACAACAGCGCCTTCATGTCTAGACTGACC |
| | | ATCAGCAAGGACACAAGCAAAAACCAGATTTCTCTCAAGAT |
| | | GAGCAGCCTGACCGCCGCTGATACAGCCGTGTATTACTGCG |
| | | CCAGAGATTGGGCCTACTGGGGCCAGGGCACCCTGGTGACC |
| | | GTGTCCTCC |
| 77 | CUSB0136- | CAAGTGCAGCTGCAGGAGAGCGGCCCTGGCCTGGTCAAGCC |
| | BMVH.3d | CAGCGAGACACTGAGCCTGACCTGTACCGTGTCCGGCTTCA |
| | | GCCTGACCGTGTACGACATCATTTGGATCCGGCAGAGCCCT |
| | | GGAAAAGGCCTGGAATGGCTGGGCGTGATCTGGACCGGCG |
| | | GAGATACAACATACAACAGCGCCTTCATGTCTAGACTGACA |
| | | ATCTCCAAGGACACCAGCAAGAACCAGATCAGCCTCAAGAT |
| | | GAGCAGCCTGACAGCCGCTGATACCGCCGTGTATTACTGCG |
| | | CCAGAGACTGGATGTACTGGGGCCAGGGCACCCTGGTGACC |
| | | GTGTCTTCT |
| 78 | CUSB0136- | GACATCGTGCTGACCCAGAGCCCCGACAGCCTGGCCGTGTC |
| | BMVL.2a | CCTGGGCGAGCGGGCCACAATTTCTTGTAGAGCTTCTGAAT |
| | | CCGTGGACAACTGGGGCAATAGCTTCATCCAGTGGTATCAG |
| | | CAGAAACCTGGCCAGCCTCCAAAGCTGCTGATCTACTTCGC |
| | | CAGCTACCTCCAATCTGGCGTCCCCGATAGATTCAGCGGAA |
| | | GCGGCAGCGGCACCGACTTCACCCTGACAATCAGCAGCGTG |
| | | CAGGCCGAGGACGTGGCCACCTACTACTGCCACCAGAACAA |
| | | CGAGGATCCTTTTACCTTCGGCGGAGGCACAAAGCTGGAAA |
| | | TCAAG |
| 79 | CUSB0136- | GACATCGTGCTGACCCAGAGCCCCGACAGCCTGGCCGTGTC |
| | BMVL.2b | CCTCGGCGAGCGGGCCACAATTTCTTGTAGAGCTTCCGAGA |
| | | GCGTGGACAACTACGGCAATAGCTTCATCCAGTGGTATCAG |
| | | CAGAAACCTGGCCAGCCTCCAAAGCTGCTGATCTACTTCGC |
| | | CAGCTACCTGCAATCTGGCGTCCCTGATAGATTCAGCGGCA |
| | | GCGGCTCTGGCACCGACTTCACCCTGACAATCAGCAGCGTG |
| | | CAGGCCGAAGATGTGGCCACCTACTACTGCCACCAGAACAA |
| | | CGAGGACCCCTTTACCTTCGGCGGAGGAACAAAGCTGGAAA |
| | | TCAAG |

The DNA encoding an antibody disclosed herein also can be modified, for example, by substituting the coding sequence for human heavy and light chain constant domains in place of the homologous murine sequences or by covalently joining to the immunoglobulin coding sequence all or part of the coding sequence for a non-immunoglobulin polypeptide. Such a non-immunoglobulin polypeptide can be substituted for the constant domains of an antibody disclosed herein, or can be substituted for the variable domains of one antigen-combining site of an antibody disclosed herein to create a chimeric bivalent antibody.

In another aspect, provided herein is a vector comprising a polynucleotide encoding a VH and/or a VL of an anti-PD-1 antibody or antigen-binding fragment thereof. In some embodiments, the vector is a viral vector. In some embodiments, the vector is a retroviral vector, for example, moloney murine leukemia viruses. In some embodiments, the vector is a pox vector, a herpes simplex I virus (HSV) vector, an adenovirus vector, or an adeno-associated virus vector.

In one embodiment, the present disclosure includes variants to the nucleic acid sequences that encode for an anti-PD-1 antibody or antigen-binding fragment thereof disclosed herein. For example, the variants include nucleotide sequences that hybridize to the nucleic acid sequences encoding an anti-PD-1 antibody or antigen-binding fragment thereof of the present disclosure under at least moderately stringent hybridization conditions.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g., 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10 [Na+])+0.41(% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions.

### Methods of Making

Using known techniques, antibodies can be generated to the anti-PD-1 antibodies or antigen-binding fragments thereof, and expression libraries thereto, which can thereafter be screened for the activities described herein.

The antibodies and fragments disclosed herein can be expressed by a vector containing a DNA segment encoding a VH and/or a VL of an anti-PD-1 antibody or antigen-binding fragment thereof, such as those described above.

Antibodies may be purified by well-known techniques, such as affinity chromatography using protein A or protein G, which provide primarily the IgG fraction of immune serum. Subsequently. or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

Various procedures known within the art may be used for the production of monoclonal antibodies directed against PD-1 or against derivatives, fragments, analogs, homologs or orthologs thereof. (See, for example, Antibodies: A Laboratory Manual, Harlow E, and Lane D, 1 988, Cold Spring Harbor Laboratory Press, Cold Spring Harbor, NY, incorporated herein by reference).

Monoclonal antibodies may be generated, e.g., by immunizing an animal with PD-1, such as, for example, murine, rat or human PD-1 or an immunogenic fragment, derivative or variant thereof. e.g., by subcutaneous injection at regular intervals. Alternatively, the animal may be immunized with cells transfected with a vector containing a nucleic acid molecule encoding PD-1 that is expressed and associated with the surface of the transfected cells. For example, mice may be immunized with PD-1 overexpressing CHO cells by intraperitoneal injection at regular intervals. Immunized animals may be monitored for serum IgG titers and the animals with the highest titers may be used for hybridoma generation. To generate hybridomas, the spleen of an immunized animal is removed and spleen cells are fused with myeloma cells, e.g., by electrofusion.

Alternatively, the antibodies are obtained by screening a library that contains antibody or antigen binding domain sequences for binding to PD-1. This library is prepared, e.g., in bacteriophage as protein or peptide fusions to a bacteriophage coat protein that is expressed on the surface of assembled phage particles and the encoding DNA sequences contained within the phage particles (i.e., “phage displayed library”). Hybridomas resulting from myeloma/B cell fusions are then screened for reactivity to PD-1.

Monoclonal antibodies may be prepared, for example, using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Monoclonal antibodies can also be made by recombinant DNA methods. DNA encoding the monoclonal antibodies and antigen-binding fragments thereof can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells.

Antibodies may be purified using any suitable method known in the art. For example, the medium of the hybridoma culture may be centrifuged and the supernatant filtered through a 0.45 μm PES membrane filter. The antibodies may then be purified using, for example, a HiTrap protein A column. Then, bound antibodies can be eluted and concentrated and buffer-exchanged to PBS.

Screening of monoclonal antibodies and antigen-binding fragments thereof, can be also carried out, e.g., by measuring binding between PD-L1 and PD-1 and determining whether the test monoclonal antibody is able to inhibit binding between PD-L1 and PD-1. Antibodies may be screened for their binding to PD-1 using any suitable method known in the art. To measure PD-1 binding, the antibody to be screened may be incubated with His-tagged PD-1 and then probed with anti-IgG Fcγ-HRP antibody. The HRP can then be detected using substrate solution and the absorbance value determined, with a higher absorbance value indicating stronger PD-1 binding.

Antibodies may also be screened for their ability to neutralize the PD-1/PD-L1 interaction. To measure PD-1/PD-L1 neutralization, the antibodies to be screened may be incubated with PD-1-expressing Jurkat effector cells and recombinant PD-L1 human Fc-tag protein. A fluorescent anti-human IgG antibody may be used to determine binding of the PD-L 1 protein to the PD-1 on the cell surface, e.g., using a flow cytometer. In the presence of a neutralizing antibody, the PD-L1 human Fc-tag protein cannot bind to the PD-1-expressed Jurkat cells resulting in the absence of a fluorescence signal.

Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Techniques can be adapted for the production of single-chain antibodies specific to an antigenic protein of the disclosure (see e.g., U.S. Pat. No. 4,946,778). In addition, methods can be adapted for the construction of Fab expression libraries (see e.g., Huse, et al., 1989 Science 246:1275-1281) to allow rapid and effective identification of monoclonal Fab fragments with the desired specificity for a protein or derivatives, fragments, analogs or homologs thereof.

Various techniques for making and isolating bispecific antibody fragments directly from recombinant cell culture have been described. For example, bispecific antibodies have been produced using leucine zippers. Kostelny et al., J. Immunol 148(5):1547-1553 (1992). The “diabody” technology described by Hollinger et al., Proc. Natl. Acad. Sci. USA 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, Gruber et al., J. Immunol. 152:5368 (1994).

The antibodies and antibody fragments described herein may be humanized in order to make them better tolerated for use in humans. For example, amino acid residues in the framework regions may be humanized by replacing them with amino acid residues and the human framework regions as long as the replacement does not impair the ability of the antibodies and antibody fragments to bind to the antigen (Vincke C, Loris R, Sacrens D, Martinez-Rodriguez S, Muyldermans S, Conrath K., “General strategy to humanize a camelid single-domain antibody and identification of a universal humanized nanobody scaffold”, J Biol. Chem. 2008).

It will be appreciated that murine antibodies or antibodies from other species can be humanized or primatized using techniques well known in the art. See e.g., Winter and Harris Immunol Today 14:43 46 (1993) and Wright et al., Crit. Reviews in Immunol. 12125-168 (1992), The antibody of interest may be engineered by recombinant DNA techniques to substitute the CH1, CH2, CH3, hinge domains, and/or the framework domain with the corresponding human sequence (See WO 92102190 and U.S. Pat. Nos. 5,530,101; 5,585,089; 5,693,761; 5,693,792; 5,714,350; and 5,777,085). Also, the use of Ig cDNA for construction of chimeric immunoglobulin genes is known in the art (Liu et al., P.N.A.S. 84:3439 (1987) and J. Immunol. 139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library is made and screened to isolate the sequence of interest. The DNA sequence encoding the variable region of the antibody is then fused to human constant region sequences. The sequences of human constant regions genes may be found in Kabat et al., (1991) Sequences of Proteins of immunological Interest, N.I.H. publication no. 91-3242. Human C region genes are readily available from known clones. The choice of isotype will be guided by the desired effecter functions, such as complement fixation, or activity in antibody-dependent cellular cytotoxicity. Optional isotypes are IgG1, IgG3 and IgG4. Either of the human light chain constant regions, kappa or lambda, may be used. The chimeric, humanized antibody is then expressed by conventional methods.

Fully human antibodies are antibody molecules in which the entire sequence of both the light chain and the heavy chain, including the CDRs, arise from human genes. Such antibodies are termed “human antibodies” or “fully human antibodies” herein. Human monoclonal antibodies are prepared, for example, using the procedures described in the Examples provided below. Human monoclonal antibodies can be also prepared by using the trioma technique; the human B-cell hybridoma technique (see Kozbor, et al., 1983 Immunol Today 4:72); and the EBV hybridoma technique to produce human monoclonal antibodies (see Cole, et al., 1985 In: Monoclonal Antibodies and Cancer Therapy, Alan R. Liss, Inc., pp. 77-96). Human monoclonal antibodies may be utilized and may be produced by using human hybridomas (see Cote, et al., 1 983. Proc Natl Acad Sci USA 80:2026-2030) or by transforming human B-cells with Epstein Barr Virus in vitro (see Cole, et al., 1985 In: Monoclonal Antibodies and Cancer Therapy, Alan R. Liss, Inc., pp. 77-96).

In some methods, an antibody or fragment is developed, for example, using phage-display methods using antibodies containing only human sequences. Such approaches are well-known in the art, e.g., in WO92/01047 and U.S. Pat. No. 6,521,404, which are hereby incorporated by reference. In this approach, a combinatorial library of phage carrying random pairs of light and heavy chains are screened using natural or recombinant source of PD-1 or fragments thereof. In another approach, an antibody or fragment can be produced by a process wherein at least one step of the process includes immunizing a transgenic, non-human animal with a PD-1 protein. In this approach, some of the endogenous heavy and/or kappa light chain loci of this xenogeneic/non-human animal have been disabled and are incapable of the rearrangement required to generate genes encoding immunoglobulins in response to an antigen. In addition, at least one human heavy chain locus and at least one human light chain locus have been stably transfected into the animal. Thus, in response to an administered antigen, the human loci rearrange to provide genes encoding human variable regions immunospecific for the antigen. Upon immunization, therefore, the animal produces B-cells that secrete fully human immunoglobulins.

In an alternative approach, others have utilized a “minilocus” approach in which an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal.

Generation of human antibodies from mice in which, through microcell fusion, large pieces of chromosomes, or entire chromosomes, have been introduced, has also been demonstrated.

Those of ordinary skill in the art will recognize that a large variety of possible moieties can be coupled to the resultant antibodies and antigen-binding fragments thereof. (See, for example, “Conjugate Vaccines”, Contributions to Microbiology and Immunology, J. M. Cruse and R. E. Lewis, Jr (eds), Carger Press, Mew York, (1989), the entire contents of which are incorporated herein by reference). Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation.

For example, conjugates of the antibody and effector molecule can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled I-isothiocyanatobenzyl-3-methyldiethylene triaminepentaacetic acid (MX-DTPA) is an exemplary chelating agent for conjugation of radionucleotide to the antibody (see WO94/11026).

### Pharmaceutical Compositions

In another aspect, provided herein are pharmaceutical compositions comprising the anti-PD-1 antibodies or antigen-binding fragments thereof and optionally a pharmaceutically acceptable carrier.

As used herein, the term “pharmaceutically acceptable carrier” is intended to include any and all solvents, dispersion media, coatings, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin.

A pharmaceutical composition is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration.

In one embodiment, the anti-PD-1 antibody or antigen-binding fragment thereof is prepared with carriers that will protect the compound against rapid elimination from the body, such as a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

In one embodiment, oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specifications for the dosage unit forms are dictated by and directly dependent on the unique characteristics of the active compound and the particular therapeutic effect to be achieved, and the limitations inherent in the art of compounding such an active compound for the treatment of individuals.

The formulation can also contain more than one active compound as necessary for the particular indication being treated, optionally those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite:chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany. N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin: an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin: or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, an anti-PD-1 antibody or antigen-binding fragment thereof is prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### Methods of Use

Also provided herein are methods of treating a disease in a subject comprising administering to the subject an anti-PD-1 antibody or antigen-binding fragment thereof provided herein, or a pharmaceutical composition provided herein. Further provided is the use of an anti-PD-1 antibody or antigen-binding fragment thereof provided herein, or a pharmaceutical composition provided herein for the treatment of cancer in a subject. Further provided is the use of an anti-PD-1 antibody or antigen-binding fragment thereof provided herein, for use in the manufacture of a medicament for the treatment of cancer in a subject.

In some embodiments, the disease to be treated is cancer. In some embodiments, the disease to be treated is a solid tumor, such as a colon cancer, breast cancer, lung cancer, ovarian cancer, prostate cancer, cervical cancer, brain cancer, skin cancer, liver cancer, pancreatic cancer or stomach cancer. In some embodiments, the cancer is a leukemia or a lymphoma. In some embodiments, the cancer has metastasized.

In some embodiments, the disease to be treated is an infectious disease, for example a chronic infectious disease such as tuberculosis, malaria, or an infection with human immunodeficiency virus (HIV) or hepatitis B virus (HBV). In some embodiments, the disease to be treated is primary immunodeficiency.

In some embodiments, a therapeutically effective amount is administered. A therapeutically effective amount of an antibody of the disclosure relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the disclosure may be, by way of non-limiting example, from about 0.1 mg kg body weight to about 50 mg/kg body weight. Common dosing frequencies may range, for example, from twice daily to once a week.

Efficaciousness of treatment is determined in association with any known method for diagnosing or treating the particular cancer. Alleviation of one or more symptoms of the cancer indicates that the antibody confers a clinical benefit.

For example, anti-tumor efficacy of humanized anti-PD-1 antibodies may be evaluated in humanized mouse models, such as the MC38 allograft mouse model. To generate such an allograft model, MC38 cells may be injected into the right flank region of a mouse, e.g., a C57BL/6-hPD-1 humanized mouse. Once the mice develop tumors of a suitable size (e.g., about 80 mm³ tumor volume on average), the anti-PD-1 antibody may be administered, for example by daily intraperitoneal injection. A suitable control such as human IgG of the same subtype as the anti-PD-1 antibody may be included. Tumor volumes may then be measured by caliper and other clinical signs, behavior, and weight loss may be recorded over the course of the study. To quantify treatment efficacy, the relative tumor growth inhibition rate may be calculated as follows: TGI %=(1−T/C)×100%, where T is tumor volume or tumor weight of treatment group and C is tumor volume or tumor weight of control group.

As used herein, “treating cancer” includes, but is not limited to, reversing, alleviating or inhibiting the progression of cancer or symptoms associated with cancer or preventing worsening of the severity of cancer or symptoms or conditions associated with cancer. “Preventing cancer” includes preventing occurrence of cancer and the prophylactic treatment of a subject in order to prevent or reduce the incidence or recurrence of cancer.

In one embodiment, the active compounds are used in combination with other therapies. Accordingly, the application provides a method of preventing or treating cancer using an anti-PD-1 antibody or antigen-binding fragment thereof disclosed herein in combination with at least one additional therapy. The other therapy may be administered prior to, overlapping with, concurrently, and/or after administration of the active compounds When administered concurrently, the anti-PD-1 antibody or antigen-binding fragment thereof and the other therapeutic may be administered in a single formulation or in separate formulations, and if separately, then optionally, by different modes of administration. The combination of one or more anti-PD-1 antibody or antigen-binding fragment thereof and one or more other therapies may synergistically act to combat cancer.

For example, the combination therapy can include an anti-PD-1 antibody or antigen-binding fragment thereof with, and/or co-administered with, one or more additional therapeutic agents, e.g., one or more cytokine and growth factor inhibitors, immunosuppressants, anti-inflammatory agents, metabolic inhibitors, enzyme inhibitors, antineoplastic agents, and/or cytotoxic or cytostatic agents. Such combination therapies may advantageously utilize lower dosages of the administered therapeutic agents, thus avoiding possible toxicities or complications associated with the various monotherapies.

In some embodiments, a subject or patient treated in accordance with a method described herein has previously undergone chemotherapy or radiation therapy. The terms “subject” and “patient” are used interchangeably herein and may refer to, for example, a living mammalian organism, such as a human, monkey, cow, horse, sheep, goat, dog, cat, mouse, rat, guinea pig, or transgenic species thereof. In preferred embodiments, a subject is human.

Also provided herein are methods of inhibiting the interaction between PD-1 and one or more of its ligands using the antibodies and antigen-binding fragments described herein. For example, an antibody or antigen-binding fragment described herein may inhibit the interaction between PD-1 and PD-L1, or between PD-1 and PD-L2. A person of skill in the art will recognize that the inhibition of the interaction between PD-1 and PD-L1 and/or between PD-1 and PD-L2 can result in beneficial effects in a cell or in an organism comprising the cell, including allowing the immune system of the organism to attack and kill tumor cells.

Thus, in one aspect, provided herein is a method of inhibiting the interaction between PD-1 and PD-L1 in a cell, comprising contacting the cell with an antibody or antigen-binding fragment thereof disclosed herein. In another aspect, provided herein is a method of inhibiting the interaction between PD-1 and PD-L2 in a cell, comprising contacting the cell with an antibody or antigen-binding fragment thereof disclosed herein. In some embodiments, the cell is a human cell. In some embodiments, the cell is a non-human cell. In some embodiments, the cell is not a human embryo. In some embodiments, the cell is contacted in vitro. In some embodiments, the cell is contacted in vivo.

In some embodiments, an antibody or antigen-binding fragment described herein inhibits the binding of PD-1 to PD-L1 with a half-maximal inhibitory concentration (IC50) of about 50 ng/mL to about 100 ng/mL, about 100 ng/mL to about 150 ng/mL, about 150 ng/mL to about 200 ng/mL, about 200 ng/mL to about 250 ng/mL, about 250 ng/mL to about 300 ng/mL, about 300 ng/mL to about 350 ng/mL, about 350 ng/mL to about 400 ng/mL, about 400 ng/mL to about 450 ng/mL, about 450 ng/mL to about 500 ng/mL, about 500 ng/mL to about 550 ng/mL, about 550 ng/mL to about 600 ng/mL, about 600 ng/mL to about 650 ng/mL, about 650 ng/mL to about 700 ng/mL, about 700 ng/mL to about 750 ng/mL, about 750 ng/mL to about 800 ng/mL, about 800 ng/mL to about 850 ng/mL, about 850 ng/mL to about 900 ng/mL, about 900 ng/mL to about 950 ng/mL, or about 950 ng/mL to about 1000 ng/mL.

In some embodiments, an antibody or antigen-binding fragment described herein inhibits the binding of PD-1 to PD-L1 with an IC50 of about 50 ng/mL, about 60 ng/mL, about 70 ng/mL, about 80 ng/mL, about 90 ng/mL about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL, about 380 ng/mL, about 390 ng/mL, about 400 ng/mL, about 410 ng/mL, about 420 ng/mL, about 430 ng/mL, about 440 ng/mL, about 450 ng/mL, about 460 ng/mL, about 470 ng/mL, about 480 ng/mL, about 490 ng/mL, or about 500 ng/mL.

In some embodiments, an antibody or antigen-binding fragment described herein inhibits the binding of PD-1 to PD-L1 with an IC50 of about 372.2 ng/mL or less. In some embodiments, an antibody or antigen-binding fragment described herein inhibits the binding of PD-1 to PD-L1 with an IC50 of about 461.0 ng/mL or less.

The IC50 for inhibition of PD-1 binding to PD-L1 may be determined using any suitable assay known in the art or described herein. Examples of such assays include the PD-1/PD-L1 blockade bioassay described in Example 5 below (see also Promega Catalogue No. J1250). In this assay, serial dilutions of the antibody to be tested are added to adherent PD-L1 expressing cells, which are subsequently incubated with effector cells (e.g., Jurkat T cells stably expressing human PD-1 and NFAT-induced luciferase) and a luminescence substrate reagent is be added to quantify the inhibition of the PD-1/PD-L1 blockade. The PD-1/PD-L1 interaction inhibits TCR signaling and NFAT-mediated luciferase activity. Addition of an antibody that blocks either PD-1 or PD-L1 releases the inhibitory signal and results in TCR signaling and NFAT-mediated luciferase activity, which can be quantified.

In some embodiments, an antibody or antigen-binding fragment described herein inhibits the binding of PD-1 to PD-L2 with an IC50 of about 50 ng/mL to about 100 ng/mL, about 100 ng/mL to about 150 ng/mL, about 150 ng/mL to about 200 ng/mL, about 200 ng/mL to about 250 ng/mL, about 250 ng/mL to about 300 ng/mL, about 300 ng/mL to about 350 ng/mL, about 350 ng/mL to about 400 ng/mL, about 400 ng/mL to about 450 ng/mL, about 450 ng/mL to about 500 ng/mL, about 500 ng/mL to about 550 ng/mL, about 550 ng/mL to about 600 ng/mL, about 600 ng/mL to about 650 ng/mL, about 650 ng/mL to about 700 ng/mL, about 700 ng/mL to about 750 ng/mL, about 750 ng/mL to about 800 ng/mL, about 800 ng/mL to about 850 ng/mL, about 850 ng/mL to about 900 ng/mL, about 900 ng/mL to about 950 ng/mL, or about 950 ng/mL to about 1000 ng/mL.

In some embodiments, an antibody or antigen-binding fragment described herein inhibits the binding of PD-1 to PD-L2 with an IC50 of about 50 ng/mL, about 60 ng/mL, about 70 ng/mL, about 80 ng/mL, about 90 ng/mL about 100 ng/mL, about 110 ng/mL, about 120 ng/mL, about 130 ng/mL, about 140 ng/mL, about 150 ng/mL, about 160 ng/mL, about 170 ng/mL, about 180 ng/mL, about 190 ng/mL, about 200 ng/mL about 210 ng/mL, about 220 ng/mL, about 230 ng/mL, about 240 ng/mL, about 250 ng/mL, about 260 ng/mL, about 270 ng/mL, about 280 ng/mL, about 290 ng/mL, about 300 ng/mL, about 310 ng/mL, about 320 ng/mL, about 330 ng/mL, about 340 ng/mL, about 350 ng/mL, about 360 ng/mL, about 370 ng/mL, about 380 ng/mL, about 390 ng/mL, about 400 ng/mL, about 410 ng/mL, about 420 ng/mL, about 430 ng/mL, about 440 ng/mL, about 450 ng/mL, about 460 ng/mL, about 470 ng/mL, about 480 ng/mL, about 490 ng/mL, or about 500 ng/mL.

In some embodiments, an antibody or antigen-binding fragment described herein inhibits the binding of PD-1 to PD-L2 with an IC50 of about 66.76 ng/mL or less.

The IC50 for inhibition of PD-1 binding to PD-L2 may be determined using any suitable assay known in the art or described herein. Examples of such assays include the flow cytometry assay described in Example 15 below. In this assay, serial dilutions of the antibody to be tested are mixed with recombinant human PD-L2 protein and the mixture is added to PD-1-expressing Jurkat effector cells. A fluorescently labeled secondary antibody may be used to detect either PD-L2 protein or test antibody bound to the PD-1-expressing Jurkat cells using flow cytometry.

Also provided herein are methods of inducing cytokine secretion in a cell using the antibodies and antigen-binding fragments described herein. Inducing secretion of cytokines in a cell may allow the cell to mediate an immune response against a disease or disorder.

Thus, in one aspect, provided herein is a method of stimulating IL-2 secretion in a cell, comprising contacting the cell with an antibody or antigen-binding fragment thereof disclosed herein. In another aspect, provided herein is a method of stimulating IFN-γ secretion in a cell, comprising contacting the cell with an antibody or antigen-binding fragment thereof disclosed herein. In some embodiments, the cell is a human cell. In some embodiments, the cell is a non-human cell. In some embodiments, the cell is not a human embryo. In some embodiments, the cell is contacted in vitro. In some embodiments, the cell is contacted in vivo.

In some embodiments, an antibody or antigen-binding fragment described herein induces the secretion of IFN-γ from T cells with a half-maximal effective concentration (EC50) of about 1 ng/mL to about 5 ng/mL, about 5 ng/mL to about 10 ng/mL, about 10 ng/mL to about 15 ng/mL, about 15 ng/mL to about 20 ng/mL, about 20 ng/mL to about 25 ng/mL, about 25 ng/mL to about 30 ng/mL, about 30 ng/mL to about 35 ng/mL, about 35 ng/mL to about 40 ng/mL, about 40 ng/mL to about 45 ng/mL, about 45 ng/mL to about 50 ng/mL, about 50 ng/mL to about 55 ng/mL, about 55 ng/mL to about 60 ng/mL, about 60 ng/mL to about 65 ng/mL, about 65 ng/mL to about 70 ng/mL, about 70 ng/mL to about 75 ng/mL, about 75 ng/mL to about 80 ng/mL, about 80 ng/mL to about 85 ng/mL, about 85 ng/mL to about 90 ng/mL, about 90 ng/mL to about 95 ng/mL, or about 95 ng/mL to about 100 ng/mL.

In some embodiments, an antibody or antigen-binding fragment described herein induces the secretion of IFN-γ from T cells with an EC50 of about 5 ng/mL, about 6 ng/mL, about 7 ng/mL, about 8 ng/mL, about 9 ng/mL about 10 ng/mL, about 11 ng/mL, about 12 ng/mL, about 13 ng/mL, about 14 ng/mL, about 15 ng/mL, about 20 ng/mL, about 25 ng/mL, about 30 ng/mL, about 35 ng/mL, about 40 ng/mL about 45 ng/mL, about 50 ng/mL, about 55 ng/mL, about 60 ng/mL, about 65 ng/mL, about 70 ng/mL, about 75 ng/mL, about 75 ng/mL, about 80 ng/mL, about 85 ng/mL, about 90 ng/mL, about 95 ng/mL, or about 100 ng/mL.

In some embodiments, an antibody or antigen-binding fragment described herein induces the secretion of IFN-γ from T cells with an EC50 of about 38.4 ng/mL or less.

In some embodiments, an antibody or antigen-binding fragment described herein induces the secretion of IL-2 from T cells with an EC50 of about 0.1 ng/mL to about 0.5 ng/mL, about 0.5 ng/mL to about 1 ng/mL, about 1 ng/mL to about 5 ng/mL, about 5 ng/mL to about 10 ng/mL, about 10 ng/mL to about 15 ng/mL, about 15 ng/mL to about 20 ng/mL, about 20 ng/mL to about 25 ng/mL, about 25 ng/mL to about 30 ng/mL, about 30 ng/mL to about 35 ng/mL, about 35 ng/mL to about 40 ng/mL, about 40 ng/mL to about 45 ng/mL, about 45 ng/mL to about 50 ng/mL, about 50 ng/mL to about 55 ng/mL, about 55 ng/mL to about 60 ng/mL, about 60 ng/mL to about 65 ng/mL, about 65 ng/mL to about 70 ng/mL, about 70 ng/mL to about 75 ng/mL, about 75 ng/mL to about 80 ng/mL, about 80 ng/mL to about 85 ng/mL, about 85 ng/mL to about 90 ng/mL, about 90 ng/mL to about 95 ng/mL, about 95 ng/mL to about 100 ng/mL, about 100 ng/mL to about 110 ng/mL, about 110 ng/mL to about 120 ng/mL, about 120 ng/mL to about 130 ng/mL, about 130 ng/mL to about 140 ng/mL, about 140 ng/mL to about 150 ng/mL, about 150 ng/mL to about 160 ng/mL, about 160 ng/mL to about 170 ng/mL, about 170 ng/mL to about 180 ng/mL, about 180 ng/mL to about 190 ng/mL, about 190 ng/mL to about 200 ng/mL, about 200 ng/mL to about 210 ng/mL, about 210 ng/mL to about 220 ng/mL, about 220 ng/mL to about 230 ng/mL, about 230 ng/mL to about 240 ng/mL, or about 240 ng/mL to about 250 ng/mL.

In some embodiments, an antibody or antigen-binding fragment described herein induces the secretion of IL-2 from T cells with an EC50 of about 0.1 ng/mL, about 0.5 ng/mL, about 1 ng/mL, about 5 ng/mL, about 10 ng/mL about 15 ng/mL, about 20 ng/mL, about 25 ng/mL, about 30 ng/mL, about 35 ng/mL, about 40 ng/mL, about 45 ng/mL, about 25 ng/mL, about 30 ng/mL, about 35 ng/mL, about 40 ng/mL about 50 ng/mL, about 60 ng/mL, about 70 ng/mL, about 80 ng/mL, about 90 ng/mL, or about 100 ng/mL.

In some embodiments, an antibody or antigen-binding fragment described herein induces the secretion of IL-2 from T cells with an EC50 of about 0.2 ng/mL or less.

The secretion of cytokines such as IFN-γ and/or IL-2 from immune cells may be determined using any suitable assay known in the art or described herein. Examples of such assays include the mixed lymphocyte reaction assay described in Example 8 below. In this assay, serial dilutions of the antibody to be tested are added to effector cells (e.g., human CD4⁺ T cells). Subsequently, antigen presenting cells (e.g., human dendritic cells) are co-cultured with the effector cells and the secreted levels of IL-2 and IFN-γ in the culture medium can be quantified using commercially available kits (e.g., the human IL-2 HTRF kit and human IFN-γ HTRF© kits from Cisbio).

### EXAMPLES

The examples below provide illustrative details of methods that may be used to generate and characterize the antibodies and antigen-binding fragments described herein. The examples are provided for illustration only and are not intended to limit the invention.

### Abbreviations and Definitions

BM Back mutation
CDR Complementarity-determining region
DPBS Dulbecco's phosphate-buffered saline
DNA Deoxyribonucleic acid
EC₅₀ Half-maximal effective concentration
ELISA Enzyme-linked immunosorbent assay
Fc Fragment crystallizable
HC Heavy chain
HRP Horseradish peroxidase
IC₅₀ Half-maximal inhibitory concentration
IgG Immunoglobulin G
LC Light chain
m (prefix) Mouse antibody
OPD O-phenylenediamine dihydrochloride
PBS Phosphate-buffered saline
PBST Phosphate-buffered saline with tween-20
RNA Ribonucleic acid
VH Variable region of heavy chain
VL Variable region of light chain
x (prefix) Chimeric antibody
z (prefix) Humanized antibody

### Example 1: Generation of Mouse Monoclonal Antibodies Against Human PD-1 Protein

Immunization of Mice with Recombinant PD-1 Proteins and CHO Overexpressing PD-1 Cells

To generate mouse monoclonal antibodies against human PD-1. BALB/c and ICR mice were immunized with either recombinant PD-1 Fc chimeric proteins and/or CHO overexpressing PD-1 cells. The PD-1 Fc chimeric protein was emulsified in AdjuLite™ Freund's complete adjuvant (first immunization) or incomplete adjuvant (booster). The emulsion mixture was injected in a 4-week interval via the subcutaneous route into the neck (50 μL) and the intramuscular route into the left and right thigh (50 μL/site). CHO overexpressing PD-1 cells were suspended in a sterile PBS buffer (1×10⁷ cells/150 μL). The cell suspension was injected in a 2-week interval via the intraperitoneal route. PD-1-specific serum IgG titers were regularly monitored. Mice with the highest IgG titers were selected for final immunization, and their spleens were collected for hybridoma generation.

### Mouse Hybridoma Generation and Selection

The selected mice were anesthetized, and a splenectomy was performed using an aseptic technique. Splenocytes were washed 3 times with serum-free medium (360×g at 25° C. for 7 min per round). Splenocytes and myeloma cells were counted using a Countess™ 3 FL automated cell counter (Invitrogen™). Then splenocytes and myeloma cells were mixed at a ratio of 2:1. The cell mixture was washed 3 times with 10 ml sterile DPBS buffer, followed by 3 times 10 ml sterile BTX buffer. The cell mixture was resuspended in the ice-cold BTX buffer (5×10⁷ cells/mL) and transferred into an electrocuvette (400 μL/cuvette). Electrofusion was performed using the BTX ECM 2001 machine with an optimized condition. Fused cells were recovered in the ClonaCell™-HY medium C (STEMCELL™) at 37° C. in a 5% CO₂ humidified incubator overnight. Recovered cells were harvested (360×g at 25° C. for 7 min) and reconstituted in the ClonaCell™-HY medium C (STEMCELL™) at 2×10⁷ cells/mL. The cell suspension was then gently mixed with ClonaCell™-HY medium D (STEMCELL™) at a ratio of 1:9 (v/v) according to the ClonaCell™-HY instruction manual. The cell mixture was plated into a 6-well cell culture plate (2.5 mL/well) and incubated at 37° C. in a 5% CO₂ humidified incubator for 7-10 days. Hybridoma colonies were randomly picked by pipetting and transferred to each well (mini-pool) of a 96-well culture plate containing 200 μL of ClonaCell™-HY medium E (STEMCELL™). After culturing for 2 days, the mini-pool supernatants were screened for PD-1 binding and neutralizing properties.

### Example 2: Selection of Mouse Hybridoma Clones Producing Potent Anti-PD-1 Antibody

### PD-1 Specific Binding Screening

ELISA technique was used to select hybridoma mini-pools producing anti-PD-1 antibodies using the following procedure. MaxiSorp ELISA plate was coated with PD-1 His-tag (R&D System) in PBS buffer (10 ng/100 μL/well) at 4° C. overnight. The plate was washed 3 times with PBST washing buffer (0.05% Tween-20 in PBS) using a BioTek EL 406 plate washer. The culture supernatant of each mini-pool (100 μL) was added to the plate then incubated for 1 hour at 37° C. After washing, the plate was probed with goat anti-mouse IgG Fcγ-HRP (Jackson Immuno Research) diluted 1:8,000 in PBST (100 μL/well) and incubated for 1 hour at 37° C. After 3 times of washing, the plate was incubated with SIGMAFAST™ OPD (Sigma-Aldrich) substrate solution (100 μL/well) in the dark for 20 min at room temperature. 1 M H₂SO₄ (50 μL/well) was used to stop the reaction. The absorbance was measured at 492 nm by the Cytation™ 5 cell imaging multi-mode reader (BioTek). Hybridoma mini-pools with absorbance values of more than 1.0 were selected for further screening.

### PD-1/PD-L1 Neutralization Screening

Flow cytometry was used to select hybridoma mini-pools producing PD-1/PD-L1 neutralizing antibodies. A 96-wells V bottom plate (Nunc™) containing PD-1-expressed Jurkat effector cells (1.5×10⁴ cells/well) in FPBS buffer (10% FBS in PBS) was incubated with the culture supernatant from the previously selected hybridoma mini-pools (100 μL) and recombinant PD-L1 human Fc-tag protein (GenScript) (0.5 μg) at room temperature for 30 min. The supernatant was discarded after centrifuging the plate at 500 g for 5 min. Alexa Fluor® 647 goat anti-human IgG (Jackson Immuno Research) diluted 1:100 in FPBS buffer was added to each well (100 μL). The plate was incubated in the dark at room temperature for 30 min, followed by washing twice with FPBS buffer. Cells were resuspended with FPBS buffer (100 μL) and analyzed by a flow cytometer (Intellicyt iQue screener plus). In the presence of a neutralizing antibody (NAb), the PD-L1 human Fc-tag protein cannot bind to the PD-1-expressed Jurkat cells resulting in the absence of a fluorescence signal. Hybridoma mini-pools with high neutralizing potency were selected for subsequently subcloning.

### Hybridoma Subcloning

Limiting dilution was performed to achieve monoclonality which was confirmed under microscopy. Then every single clone was evaluated for PD-1 specific binding and PD-1/PD-L1 neutralization, using the procedures above. Seven single hybridoma clones were finally chosen based on their high performance in both aspects of PD-1 specific binding and PD-1/PD-L1 neutralization.

### Example 3: Production and Purification of Mouse Anti-PD-1 Antibodies

Mouse anti-PD-1 antibodies were produced by culturing each hybridoma clone in HyClone SFM4MAb serum-free medium (Cytiva™) using a static condition at 37° C., 5% CO₂ for 5-7 days. The culture supernatant was harvested by centrifugation at 4000 g, 25° C. for 10 min. The supernatant was then mixed with 10× binding buffer to achieve 1× concentration (100 mM HEPES, 150 mM NaCl, pH 8.0) and filtered through a 0.45 μm PES membrane filter. The antibodies were purified using HiTrap protein A FF column (Cytiva™) by AKTA purification system. Then, bound antibodies were eluted using Pierce™ IgG elution buffer (Thermo Scientific™) then neutralized with 3 M Tris buffer. Eluted antibodies were then concentrated and buffer-exchanged to PBS using the Amicon® Ultra-15 (30K) centrifugal filter (Millipore). Antibody quantification was measured using spectrophotometry.

### Example 4: Binding Profiles of Mouse Anti-PD-1 Antibodies

The purified antibodies were 2-fold serial diluted for eleven rounds starting from 2 μg/mL until reaching the final concentration of 0.977 ng/mL. The PD-1 binding activity of purified antibodies was evaluated by ELISA following the procedure mentioned in Example 2. The half-maximal effective concentration (EC₅₀) was calculated using GraphPad Prism® software.

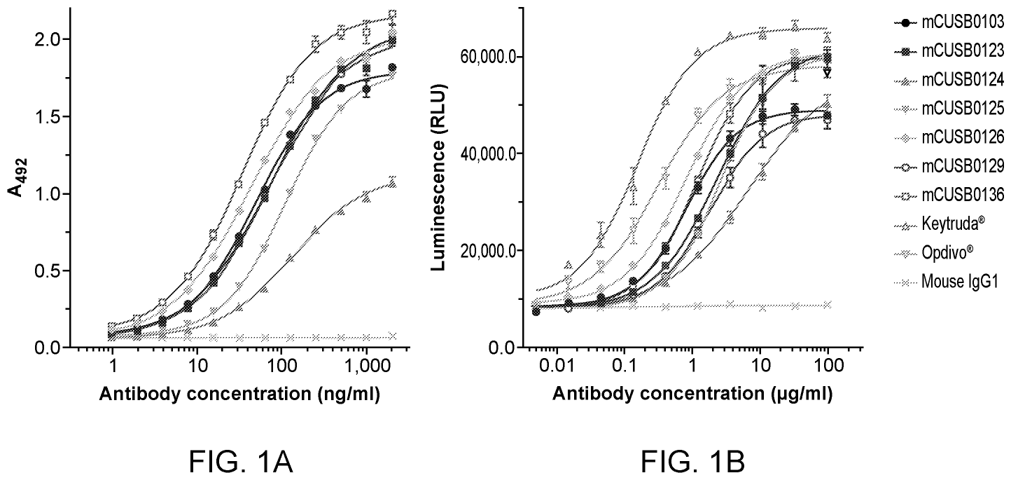

ELISA binding profiles of the mouse anti-PD-1 antibodies are illustrated in FIG. 1A. The EC₅₀ values of these antibodies are reported in Table 3.

### Example 5: PD-1/PD-L1 Neutralization Profiles of Mouse Anti-PD-1 Antibodies

The PD-1/PD-L1 blockade bioassay (Promega) was performed to evaluate the PD-1/PD-L1 neutralizing activity of the purified antibodies according to the manufacturer's instruction. In brief, the adherent PD-L1 aAPC/CHO-K1 cell was seeded in the assay plate and incubated at 37° C. in 5% CO₂ overnight. On the assay day, the medium was removed from the wells. Then, antibody samples were 3-fold serial diluted for nine rounds starting from 100 μg/ml until reaching the final concentration of 5.08 ng/mL and then incubated in the assay plate with the Jurkat/PD-1 effector cells at 37° C. in a 5% CO₂ for 6 hours. The Bio-Glo™ substrate reagent (Promega) was added to the assay plate and incubated at room temperature for 5 min. Luminescence signals were measured using the Cytation™ 5 cell imaging multi-mode reader and reported as a relative light unit (RLU). The half-maximal inhibitory concentration (IC₅₀) was calculated using GraphPad Prism® software. PD-1/PD-L1 neutralization profiles are presented in FIG. 1B. IC₅₀ values are shown in Table 3.

TABLE 3
Binding characteristics of mouse anti-PD-1 antibodies

| | PD-1 binding ELISA, | PD-1/PD-L1 blockade |
|---|---|---|
| Antibody | EC₅₀ (ng/mL) | bioassay, IC₅₀ (μg/mL) |
| mCUSB0103 | 49.4 | 0.843 |
| mCUSB0123 | 70.1 | 2.433 |
| mCUSB0124 | 103.9 | 6.578 |
| mCUSB0125 | 109.4 | 2.706 |
| mCUSB0126 | 44.2 | 0.846 |
| mCUSB0129 | 74.0 | 1.830 |
| mCUSB0136 | 34.2 | 1.212 |

### Example 6: Sequencing of Mouse Antibody Variable Regions

Total RNA was extracted from hybridoma cells using TRIzol® Reagent then reverse-transcribed into cDNA using isotype-specific anti-sense primers or universal primers following the technical manual of PrimeScript™ 1^{st} Strand cDNA Synthesis Kit. Variable fragments of antibody heavy chain and light chain were amplified using the rapid amplification of cDNA ends (RACE) technique. Amplicons were cloned into a standard cloning vector and transformed into E. coli Top10. Colony PCR was performed to select clones with correct insert sizes. Variable fragment sequences were analyzed by Sanger sequencing. The nucleotide sequences and amino acid sequences are shown in Table 2 and Table 1, respectively.

### Example 7: Chimerization of Anti-PD-1 Antibodies

### Antibody Chimerization

The variable regions of mouse anti-PD-1 antibodies were joined to human IgG4/kappa constant regions (based on the human kappa constant region [UniProt accession number: P01834] and human IgG4 constant region [UniProt accession number: P01861]). Chimeric heavy and light chains were synthesized and separately cloned into the pcDNA3.4 expression vector.

### Antibody Production and Purification

A small-scale transient expression of the chimeric antibodies was performed using the Expi293™ expression system. Briefly, 15 μg of sterile transfection grade expression vectors (LC:HC ratio of 2:1) were mixed with diluted ExpiFectamine™ 293 solution. Fifteen milliliters of Expi293F™ cell suspension (3×10⁶ cells/mL) was added with the mixture solution then incubated at 37° C., 8% CO₂, shaking for 7 days. The culture medium was harvested, and the chimeric antibody was purified using the method described in Example 3.

### Example 8: In Vitro Characterization of Chimeric Anti-PD-1 Antibodies

### Binding Profile of Chimeric Anti-PD-1 Antibodies

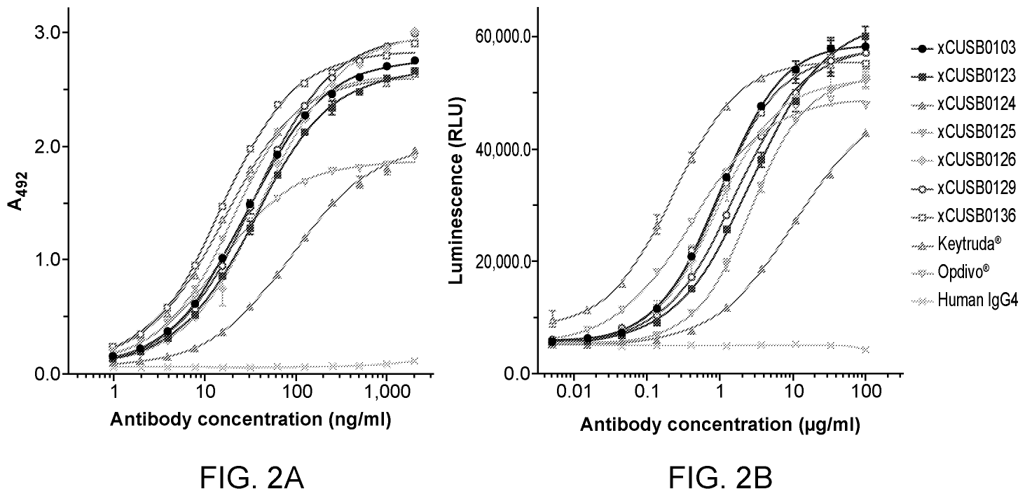

The purified chimeric antibodies were 2-fold serial diluted for eleven rounds starting from 2 g/ml until reaching the final concentration of 0.977 ng/mL. The PD-1 binding activity of purified chimeric antibodies was evaluated by ELISA following the procedure mentioned in Example 2, while the goat anti-human IgG Fcγ-HRP antibody (Jackson Immuno Research) diluted 1:10.000 in PBST was used to detect chimeric antibodies. ELISA binding profiles of the chimeric anti-PD-1 antibodies are illustrated in FIG. 2A. The EC₅₀ values of chimeric antibodies are shown in Table 4.

### PD-1/PD-L1 Neutralizing Profile of Chimeric Anti-PD-1 Antibodies

To evaluate the PD-1/PD-L1 neutralizing profile, PD-1/PD-L1 blockade bioassay was performed as previously described in Example 5. PD-1/PD-L1 neutralization profiles of chimeric antibodies are presented in FIG. 2B. The IC₅₀ values of chimeric antibodies are shown in Table 4.

### Binding Kinetics of Chimeric Anti-PD-1 Antibodies

The Biacore T200 instrument, equipped with a Protein G sensor chip (GE Healthcare), was used to determine the binding kinetics between chimeric anti-PD-1 antibodies and PD-1 protein. Each purified chimeric anti-PD-1 antibody (1 μg/mL) was injected into the chip with a flow rate of 30 μL/min for 60 sec. A single-cycle binding kinetics analysis was performed by sequentially injecting recombinant human PD-1 His-tag protein (R&D Systems) at 1.5625, 3.125, 6.25, 12.5, and 25 nM with the interval cycles of association time for 60 sec and dissociation time for 120 sec. The HBS-EP+buffer (GE Healthcare) was used as a buffer blank and diluent. The association constant (kₒₙ), dissociation constant (k_{off}), and equilibrium constant (K_{D}) values were calculated using Biacore T200 evaluation software (version 3.1) with a 1:1 Langmuir binding model curve fitting. Binding kinetic values of chimeric anti-PD-1 antibodies are shown in Table 4.

### Mixed Lymphocyte Reaction (MLR) Assay of Chimeric Anti-PD-1 Antibodies

The two-way mixed lymphocyte reaction (MLR) assay was performed to determine the activation of human T cells by chimeric antibodies. Human dendritic cells (DCs) were used as antigen-presenting cells (APCs) and human CD4⁺ T cells were used as effector cells. Serial dilutions of antibody samples starting from 10 μg/mL were added to the assay plate containing effector cells. The derived DCs were then added to the assay plate, gently mixed, and co-cultured at 37° C. in a 5% CO₂ for 3 days. The secreted levels of human interleukin 2 (IL-2) and interferon-γ (IFN-γ) were quantified using Human IL-2 HTRF kit (Cisbio) and Human IFN-γ HTRF kit (Cisbio), respectively. Commercial anti-PD-1 antibodies (Keytruda® and Opdivo®) were used as a positive control, while human IgG4 isotype control was used as a negative control. The EC₅₀ values of antibody-mediated IL-2 and IFN-γ secretion are reported in Table 4. The result showed that the two chimeric clones, xCUSB0125 and xCUSBO136, significantly induced IL-2 and IFN-γ secretion and were further selected to generate humanized anti-PD-1 antibodies.

TABLE 4
Characteristics of chimeric anti-PD-1 antibodies

| | PD-1 | PD-1/PD-L1 | | | | Mixed lymphocyte | |
|---|---|---|---|---|---|---|---|
| | binding | blockade | | | | reaction (MLR) assay | |
| | ELISA, | bioassay, | Binding kinetics by SPR | | | IFN-γ | IL-2 |
| | EC₅₀ | IC₅₀ | kₒₙ | k_{off} | K_{D} | EC₅₀ | EC₅₀ |
| Antibody | (ng/mL) | (ng/mL) | (1/Ms) | (1/s) | (nM) | (ng/mL) | (ng/mL) |
| xCUSB0103 | 28.41 | 593.4 | 6.53E+05 | 2.92E−04 | 0.448 | 13.6 | 55.7 |
| xCUSB0123 | 35.33 | 956.7 | 2.98E+05 | 3.66E−04 | 1.23 | NA | NA |
| xCUSB0124 | 89.36 | 2,929 | 1.54E+05 | 9.12E−04 | 5.92 | NA | NA |
| xCUSB0125 | 20.10 | 301.9 | 2.84E+06 | 5.12E−04 | 0.180 | 5.6 | 21.2 |
| xCUSB0126 | 43.69 | 429.6 | 4.93E+05 | 2.85E−04 | 0.577 | 77.7 | 219.1 |
| xCUSB0129 | 34.71 | 454.8 | 4.71E+05 | 2.93E−04 | 0.621 | 78.0 | 244.6 |
| xCUSB0136 | 15.41 | 372.2 | 2.30E+06 | 1.93E−04 | 0.083 | 38.4 | 0.2 |
| Keytruda ® | 15.72 | 227.7 | 6.71E+05 | 4.019E−3 | 5.983 | 5.0 | 10.9 |
| Opdivo ® | 14.57 | 460.9 | 1.92E+05 | 1.397E−3 | 7.274 | 9.1 | 44.2 |

Note:
NA; Not available

The functional characterizations of chimeric anti-PD-1 monoclonal antibodies demonstrate that these chimeric anti-PD-1 antibodies showed high PD-1 binding activity and high PD-1/PD-L1 binding blockade activity and could activate human T cells.

### Example 10: Humanization of Anti-PD-1 Antibodies

### Antibody Humanization

The CDR grafting technique was used for antibody humanization. In brief, the CDRs of xCUSB0125 and xCUSB0136 were grafted into the best-fit human immunoglobulin germlines. IGHV4-30-4*01 and IGKV1-12*01 were selected for xCUSB0125. IGHV4-59*01 and IGKV4-1*01 were selected for xCUSB0136. The back-mutation was performed in the antibody frameworks to restore the affinity of the humanized antibodies. 5 and 3 versions of variable heavy and light chains were generated from xCUSB0125. While 6 and 3 versions of variable heavy and light chains were generated from xCUSBO136. The human IgG4 S228P and kappa constant regions were used as a backbone for all humanized antibody versions. Multiple versions of each humanized antibody were generated from all combinations of its heavy and light chain versions. All the nucleotide and amino acid sequences are shown in Table 1 and Table 3, respectively.

### Antibody Production and Purification

Each version of humanized antibody was transiently expressed using the Expi293™ expression system as mentioned in Example 7. Secreted antibodies in the culture supernatant were screened for the PD-1 binding kinetics to rank the K_{D} value of each antibody version using the Biacore 8K instrument. Versions of humanized antibodies providing high binding kinetics against human PD-1 protein were selected, produced, and purified for further characterizations. Pairs of the heavy chain and light chain of selected humanized antibody versions are shown in Table 5.

TABLE 5
Pairs of heavy chain and light chain
of selected humanized antibodies

Combination of

| | Heavy chain SEQ ID | Light chain SEQ ID |
|---|---|---|
| Antibody | (DNA/AA) | (DNA/AA) |
| zCUSB0125.3 | 57/136 | 64/143 |
| zCUSB0125.6 | 58/137 | 64/143 |
| zCUSB0125.9 | 59/138 | 64/143 |
| zCUSB0125.12 | 60/139 | 64/143 |
| zCUSB0136.4 | 66/145 | 71/150 |
| zCUSB0136.5 | 66/145 | 72/151 |
| zCUSB0136.6 | 66/145 | 73/152 |
| zCUSB0136.7 | 67/146 | 71/150 |
| zCUSB0136.8 | 67/146 | 72/151 |
| zCUSB0136.9 | 67/146 | 73/152 |
| zCUSB0136.8-1 | 74/153 | 78/157 |
| zCUSB0136.8-2 | 75/154 | 79/158 |
| zCUSB0136.8-3 | 76/155 | 79/158 |
| zCUSB0136.8-4 | 77/156 | 79/158 |

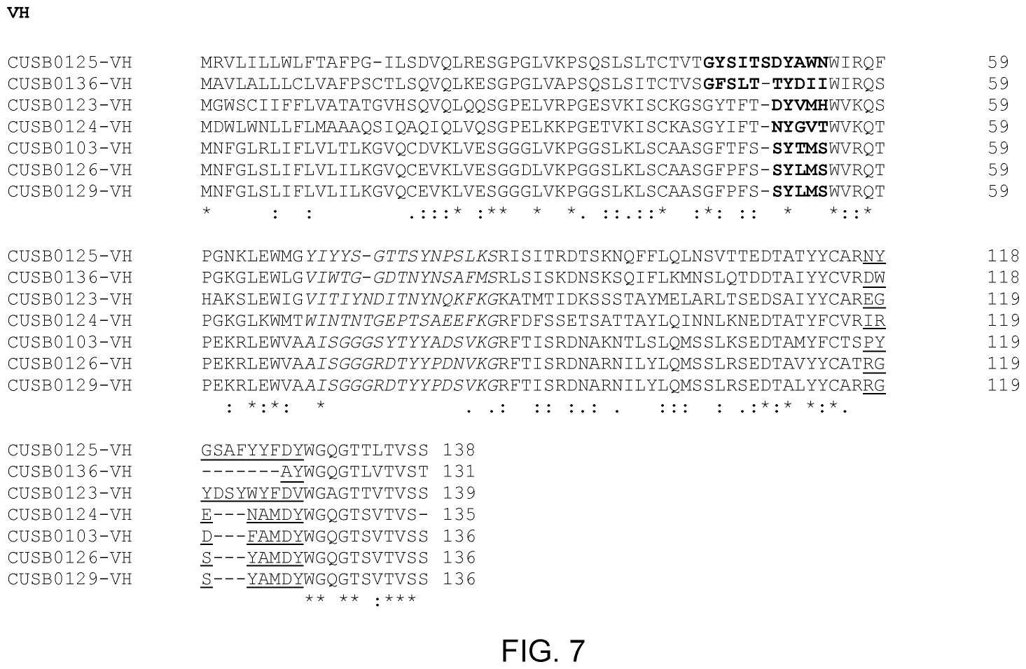

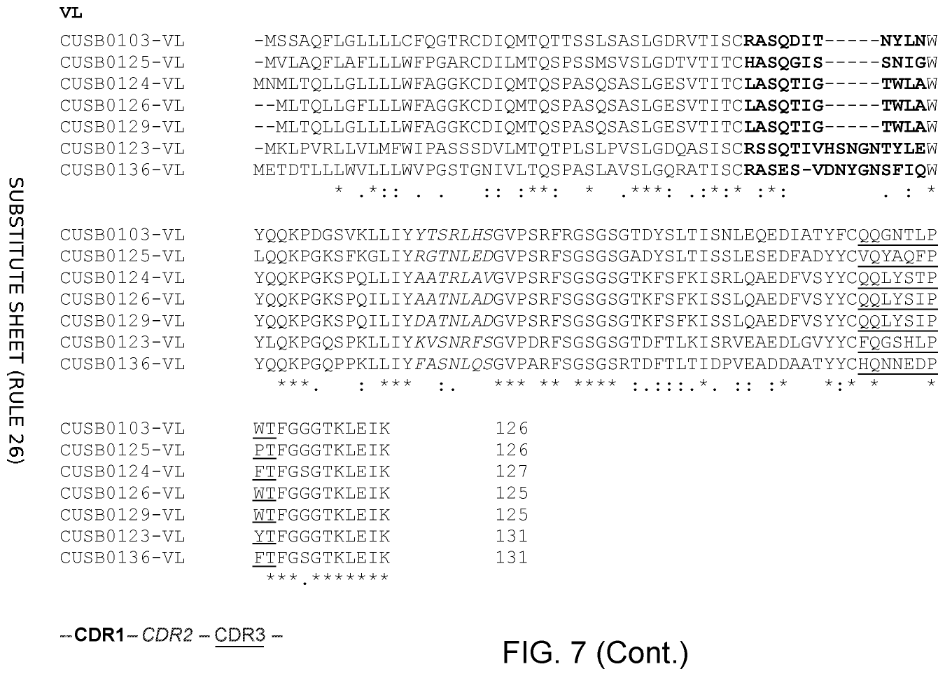

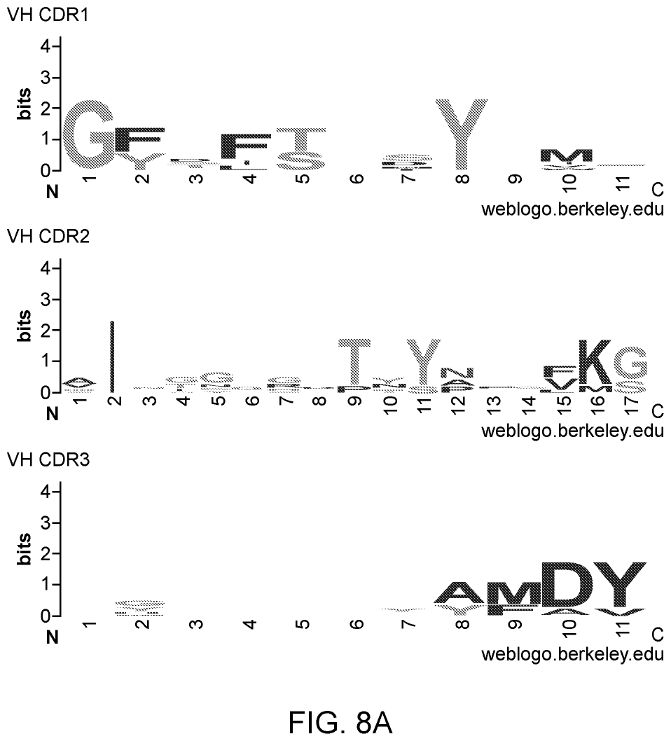

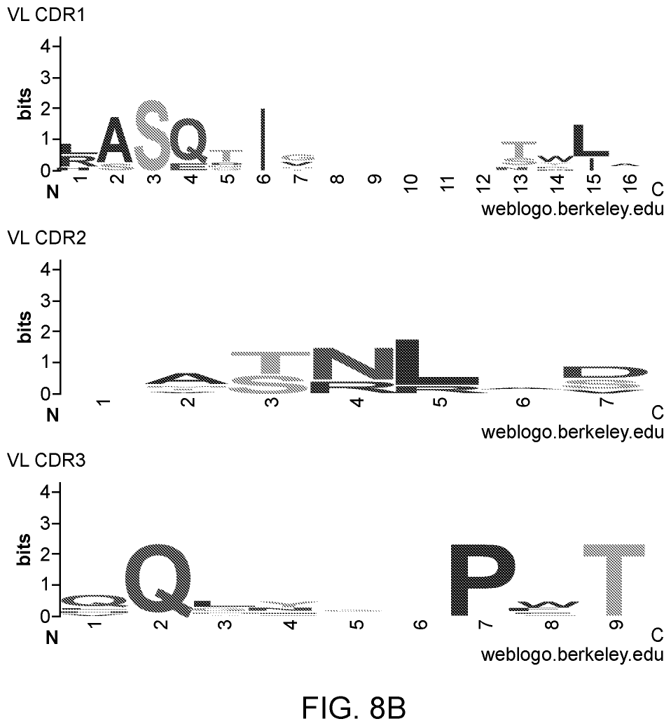

FIG. 7 shows sequence alignments of some of the anti-PD-1 antibodies described herein. FIGS. 8A and 8B show sequence logo analysis of the VH and VL of some of the anti-PD-1 antibodies described herein. Sequence logo analysis was carried out using Weblogo Version 2.8. 2 available at https://weblogo.berkeley.edu/logo.cgi.

In conclusion, four and six humanized anti-PD-1 antibodies were generated from xCUSB0125 and xCUSB0136 antibody clones, respectively.

### Example 11: In Vitro Characterization of Humanized Anti-PD-1 Antibodies

### PD-1/PD-L1 Neutralizing Profile of Humanized Anti-PD-1 Antibodies

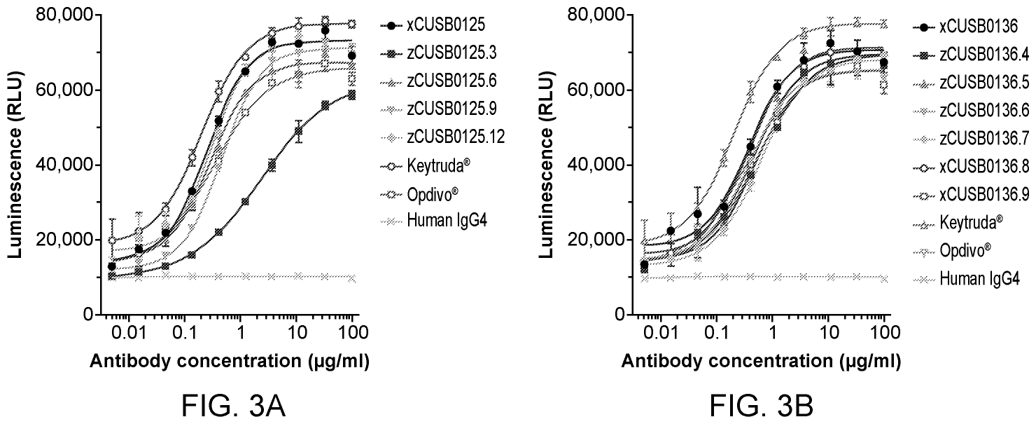

To determine the PD-1/PD-L1 neutralizing profile of humanized anti-PD-1 antibodies, the PD-1/PD-L1 blockade bioassay was performed as described in Example 5. The PD-1/PD-L1 neutralizing profiles of top-rank humanized anti-PD-1 antibodies are presented in FIG. 3. The IC₅₀ values of these antibodies are shown in Table 6.

### Binding Kinetics of Humanized Anti-PD-1 Antibodies

The Biacore T200 was used to confirm the binding kinetics of top-rank humanized anti-PD-1 antibodies as previously described in Example 8. Binding kinetic values of humanized anti-PD-1 antibodies are shown in Table 6.

### Mixed Lymphocyte Reaction (MLR) Assay of Humanized Anti-PD-1 Antibodies

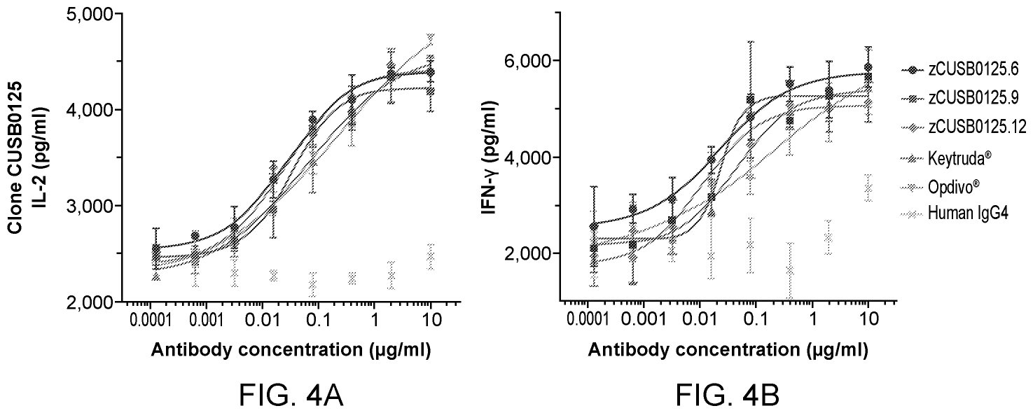

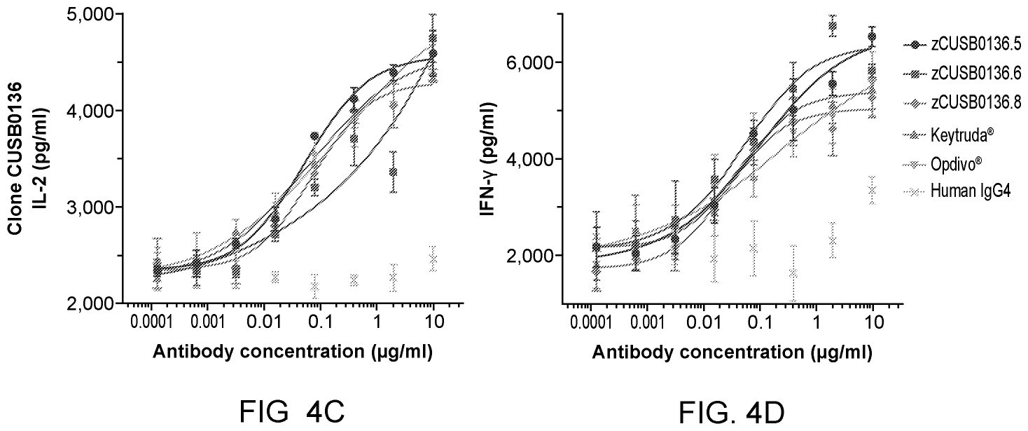

The two-way MLR assay was performed to evaluate the activation of human T cells by humanized antibodies, as described in Example 8. IL-2 and IFN-γ secretion from human T cells after the stimulation with antibodies are shown in FIG. 4. The EC₅₀ values of antibody-mediated IL-2 and IFN-γ secretion are reported in Table 6.

TABLE 6
Binding and neutralizing properties of humanized anti-PD-1 antibodies

| | | | | PD-1/PD-L1 | Mixed lymphocyte | |
|---|---|---|---|---|---|---|
| | | | | blockade | reaction (MLR) assay | |
| | Binding kinetics by SPR | | | bioassay, | IFN-γ, | IL-2, |
| | kₒₙ | k_{off} | K_{D} | IC₅₀ | EC₅₀ | EC₅₀ |
| Antibody | (1/Ms) | (1/s) | (nM) | (ng/mL) | (ng/mL) | (ng/mL) |
| zCUSB0125.3 | 2.88E+06 | 9.44E−03 | 3.280 | 2,407.0 | NA | NA |
| zCUSB0125.6 | 3.18E+06 | 7.21E−04 | 0.227 | 296.6 | 23.0 | 28.0 |
| zCUSB0125.9 | 2.99E+06 | 7.46E−04 | 0.249 | 462.1 | 23.0 | 34.0 |
| zCUSB0125.12 | 3.18E+06 | 5.79E−04 | 0.182 | 312.2 | 10.0 | 25.0 |
| zCUSB0136.4 | 1.78E+06 | 7.46E−04 | 0.420 | 547.0 | NA | NA |
| zCUSB0136.5 | 1.79E+06 | 3.70E−04 | 0.207 | 382.4 | 96.0 | 53.0 |
| zCUSB0136.6 | 1.91E+06 | 3.88E−04 | 0.204 | 375.2 | 54.0 | ND |
| zCUSB0136.7 | 1.59E+06 | 7.85E−04 | 0.493 | 613.5 | NA | NA |
| zCUSB0136.8 | 1.70E+06 | 4.26E−04 | 0.251 | 461.0 | 26.0 | 72.0 |
| zCUSB0136.9 | 1.77E+06 | 3.85E−04 | 0.218 | 492.2 | NA | NA |
| zCUSB0136.8-1 | 6.00E+06 | 1.42E−04 | 0.0237 | NA | NA | NA |
| zCUSB0136.8-2 | 4.68E+06 | 7.27E−05 | 0.0155 | NA | NA | NA |
| zCUSB0136.8-3 | 4.48E+06 | 9.31E−05 | 0.0208 | NA | NA | NA |
| zCUSB0136.8-4 | 4.42E+06 | 7.02E−05 | 0.0159 | NA | NA | NA |
| Keytruda ® | 6.71E+05 | 4.019E−03 | 5.983 | 329.0 | 45.0 | 63.0 |
| Opdivo ® | 1.92E+05 | 1.397E−03 | 7.274 | 206.1 | 239 | 225 |

Note:
NA; Not assessed;
ND; Non detectable

The functional characterizations of selected humanized anti-PD-1 antibodies reveal that each top 3 versions from zCUSB0125 and zCUSB0136 clones showed high PD-1 binding activity and high PD-1/PD-L1 binding blockade activity and could activate human T cells.

### Impact of Amino Acid Modifications on Binding Kinetics

The impact of specific amino acid modifications on binding kinetics in the zCUSB0136.8 antibody were evaluated and results are shown in Table 7.

TABLE 7
Binding Kinetics of modified zCUSB0136.8 Antibodies; numbering
is based on the VH and VL sequences shown in Table 1.

| | Modified amino acid residues | | | | | | | |
|---|---|---|---|---|---|---|---|---|
| | VH | | | VL | | Binding kinetic | | |
| | 31 | 58 | 100 | 32 | 57 | ka (1/Ms) | kd (1/s) | KD (M) |
| zCUSB0136.8 | T | N | A | Y | N | 1.87E+06 | 3.33E−04 | 1.79E−10 |
| Modified Seq1 | . | W | . | W | Y | 5.21E+06 | 2.81E−05 | 5.39E−12 |
| Modified Seq2 | V | T | I | . | Y | 4.44E+06 | 2.53E−05 | 5.71E−12 |
| Modified Seq3 | . | T | I | W | Y | 5.67E+06 | 5.28E−05 | 9.31E−12 |
| Modified Seq4 | V | T | . | . | Y | 4.15E+06 | 3.92E−05 | 9.44E−12 |
| Modified Seq5 | V | T | M | . | Y | 4.06E+06 | 4.36E−05 | 1.08E−11 |
| Modified Seq6 | . | T | I | . | Y | 4.00E+06 | 5.45E−05 | 1.36E−11 |
| Modified Seq7 | . | T | M | . | Y | 3.84E+06 | 5.48E−05 | 1.42E−11 |
| Modified Seq8 | . | W | I | . | Y | 4.13E+06 | 6.09E−05 | 1.47E−11 |
| Modified Seq9 | . | . | . | . | Y | 3.78E+06 | 5.98E−05 | 1.58E−11 |
| Modified Seq10 | . | W | M | W | H | 3.87E+06 | 6.74E−05 | 1.74E−11 |
| Modified Seq11 | . | T | . | . | Y | 3.67E+06 | 7.57E−05 | 2.06E−11 |
| Modified Seq12 | V | T | I | W | H | 3.03E+06 | 6.81E−05 | 2.25E−11 |
| Modified Seq13 | . | T | . | . | Y | 3.49E+06 | 8.47E−05 | 2.43E−11 |
| Modified Seq14 | . | T | I | W | . | 2.67E+06 | 6.50E−05 | 2.44E−11 |
| Modified Seq15 | V | T | . | W | . | 2.56E+06 | 6.30E−05 | 2.46E−11 |
| Modified Seq16 | . | T | . | . | Y | 3.50E+06 | 9.31E−05 | 2.66E−11 |
| Modified Seq17 | V | T | . | . | Y | 2.56E+06 | 7.39E−05 | 2.89E−11 |
| Modified Seq18 | . | T | . | W | Q | 2.54E+06 | 7.82E−05 | 3.07E−11 |
| Modified Seq19 | . | T | . | W | . | 2.53E+06 | 7.97E−05 | 3.15E−11 |
| Modified Seq20 | V | T | I | . | H | 2.73E+06 | 8.70E−05 | 3.19E−11 |
| Modified Seq21 | . | T | . | W | . | 2.52E+06 | 1.04E−04 | 4.12E−11 |
| Modified Seq22 | . | T | . | W | . | 2.36E+06 | 1.34E−04 | 5.70E−11 |
| Modified Seq23 | V | . | . | W | Y | 4.96E+06 | 2.88E−04 | 5.82E−11 |
| Modified Seq24 | V | T | . | . | Q | 1.81E+06 | 1.12E−04 | 6.18E−11 |
| Modified Seq25 | . | T | . | . | H | 2.18B+06 | 1.45E−04 | 6.64E−11 |

In conclusion, these specific amino acid modifications on VH and VL of zCUSB0136.8 increase its binding affinity.

### Example 12: In Vivo Anti-Tumor Efficacy of Humanized Anti-PD-1 Antibodies

### Anti-Tumor Efficacy of Humanized Anti-PD-1 Antibodies

Based on available functional property data of humanized anti-PD-1 antibodies, zCUSB0125.6 and zCUSB0136.8 were selected for further studies. In addition, the heavy chain Fc region of zCUSB0125.6 and zCUSB0136.8 anti-PD-1 antibodies was changed from IgG4 (S228P) to IgG1-LALA (L234AL235A) to modulate the binding of the antibody Fc-hinge region to Fc gamma receptors (FcγRs) which might exert an impact on antibody function and in vivo efficacy^{9,10,11,12}.

MC38 allograft/hPD-1 humanized mice model was generated by inoculating MC38 colorectal cancer cell line (2×10⁶ cells/0.1 mL, cell viability >99%) subcutaneously on the right flank region of C57BL/6-hPD-1 humanized mice. Randomized grouping was conducted when the average tumor volume reached approximately 80 mm³ for the anti-tumor evaluation. zCUSB0125.6-LALA, zCUSB0136.8-LALA, Keytruda®, and Opdivo® were used as treatment agents, while human IgG1 isotype control was used as a negative control. All treatments were performed by intraperitoneal injection every 4 days for 12 days.

Tumor volume was measured twice per week. Caliper was used to measure the length (L) and the width (W) of the tumor block. The tumor volume formula is: V=(L×W×W)/2, where V is tumor volume (mm³), L is tumor length (mm), and W is tumor width (mm).

Routine monitoring includes the effects of tumor growth and treatment on the behavior of the animals, weight gain or loss, and other abnormalities. Any clinical signs observed during the experiments would be all recorded.

### Efficacy Evaluation Criteria

The relative tumor growth inhibition rate (TGI) was calculated as follows: TGI %=(1−T/C)×100%, where T is tumor volume or tumor weight of treatment group and C is tumor volume or tumor weight of control group.

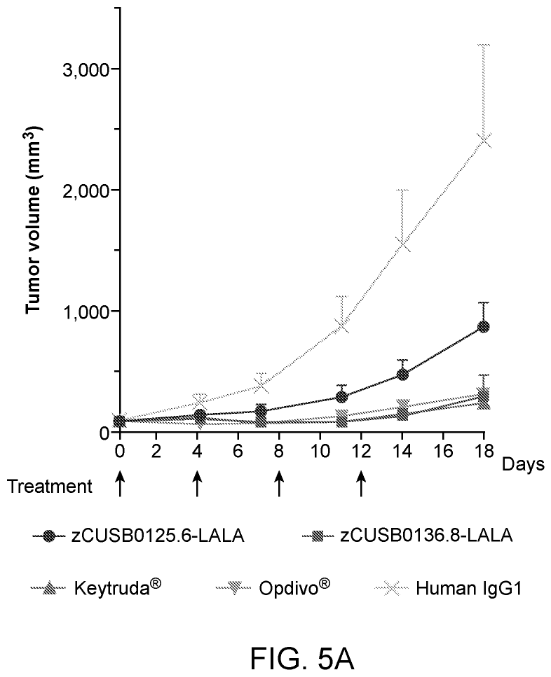

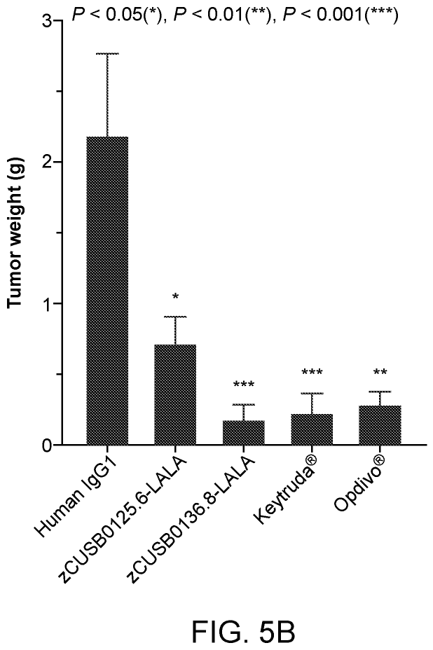

To evaluate anti-tumor efficacy of zCUSB0125.6-LALA and zCUSB0136.8-LALA anti-PD-1 antibodies, MC38 allograft/hPD-1 humanized mice (n=5 per group) were treated with 10 mg/kg every 4 days (day 0, 4, 8, and 12). The tumor volume was monitored on days 0, 4, 7, 11, 14, and 18. All mice were sacrificed on day 18 to measure the tumor weight. Tumor volume and tumor weight monitoring data are illustrated in FIG. 5. The TGI % was calculated as presented in Table 8.

TABLE 8
Anti-tumor efficacy of humanized anti-PD-1 monoclonal
antibodies compared with commercial antibodies

| | Measurement on day 18 | | | |
|---|---|---|---|---|
| Antibody | Tumor weight | TGI % (by | Tumor volume | TGI % (by |
| (10 mg/kg) | (g) | weight) | (mm³) | volume) |
| Human IgG1 | 2.19 ± 0.58 | 0.00 | 2407.53 ± 787.73 | 0.00 |
| zCUSB0125.6-LALA | 0.72 ± 0.19 | 67.12 | 869.20 ± 203.12 | 63.90 |
| zCUSB0136.8-LALA | 0.18 ± 0.11 | 91.78 | 300.48 ± 177.38 | 87.52 |
| Keytruda ® | 0.23 ± 0.14 | 89.50 | 242.89 ± 147.91 | 89.91 |
| Opdivo ® | 0.29 ± 0.09 | 86.76 | 321.38 ± 86.55 | 86.65 |

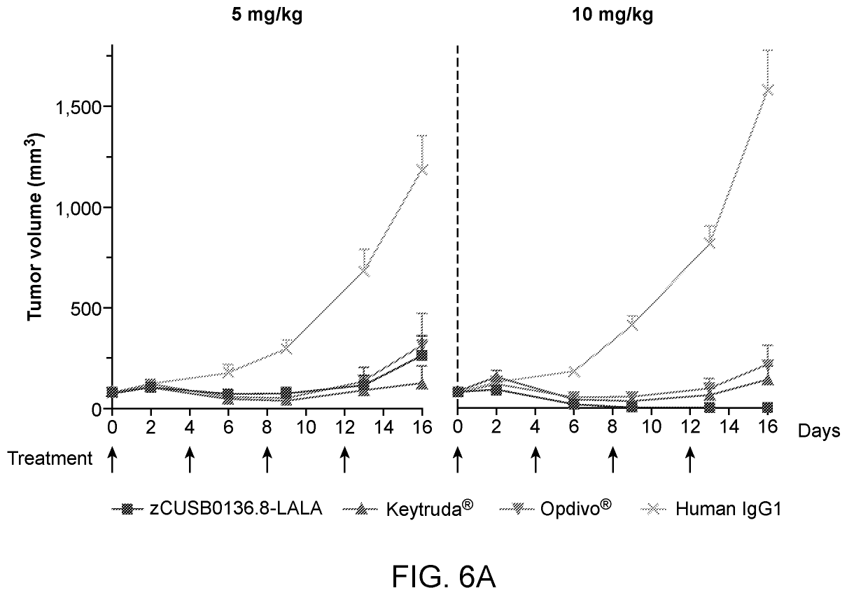

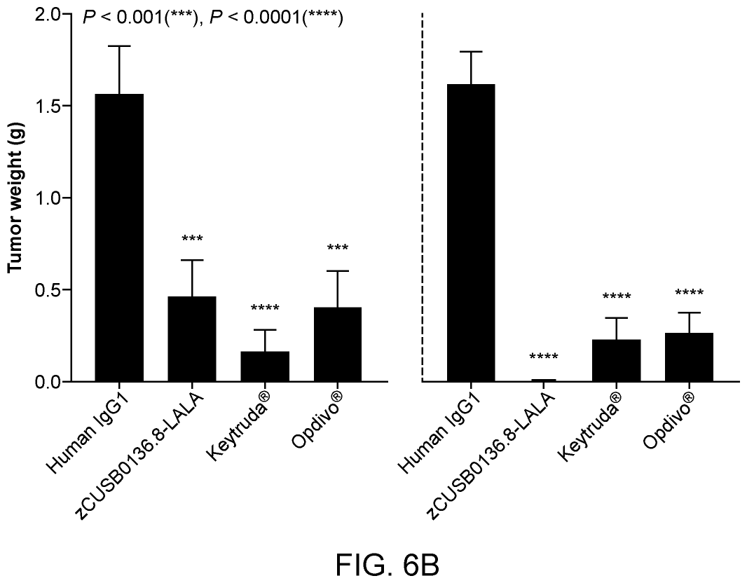

The zCUSB0136.8-LALA exhibits superior anti-tumor activity compared with zCUSB0125.6-LALA. So, the zCUSB0136.8-LALA was selected to confirm the anti-tumor activity with different dosages. MC38 allograft/hPD-1 humanized mice (n=10 per group) were treated with antibodies at low and high doses (5 and 10 mg/kg) on days 0, 4, 8, and 12. The tumor volume was monitored on days 0, 2, 6, 9, 13, and 16. All mice were sacrificed on day 16 to measure the tumor weight. Tumor volume and tumor weight are illustrated in FIG. 6. The TGI % was calculated and presented in Table 9.

TABLE 9
Anti-tumor efficacy of zCUSB0136.8-LALA compared with
commercial antibodies at the end of the study (Day 16)

| | At dose 5 mg/kg | | | | At dose 10 mg/kg | | | |
|---|---|---|---|---|---|---|---|---|
| | Tumor | TGI % | Tumor | TGI % | Tumor | TGI % | Tumor | TGI % |
| | weight | (by | volume | (by | weight | (by | volume | (by |
| Antibody | (g) | weight) | (mm³) | volume) | (g) | weight) | (mm³) | volume) |
| Human IgG1 | 1.56 ± | — | 1185.95 ± | — | 1.62 ± | — | 1581.28 ± | — |
| | 0.26 | | 171.03 | | 0.19 | | 196.31 | |
| zCUSB0136.8- | 0.47 ± | 69.87 | 262.51 ± | 77.87 | 0.003 ± | 99.81 | 0.49 ± | 99.87 |
| LALA | 0.19 | | 100.12 | | 0.002 | | 0.16 | |
| Keytruda ® | 0.17 ± | 89.10 | 123.96 ± | 89.55 | 0.23 ± | 85.80 | 139.46 ± | 91.18 |
| | 0.11 | | 88.25 | | 0.12 | | 69.88 | |
| Opdivo ® | 0.41 ± | 73.72 | 316.64 ± | 73.30 | 0.27 ± | 83.33 | 212.24 ± | 86.58 |
| | 0.2 | | 158.15 | | 0.11 | | 103.19 | |

In summary, zCUSB0136.8-LALA exhibited higher anti-tumor efficacy compared with zCUSB0125.6-LALA in MC38 allograft/hPD-1 humanized mice.

### Example 13: In Vivo Anti-Tumor Efficacy of Affinity-Matured Humanized Anti-PD-1 Antibody

### Anti-Tumor Efficacy of Affinity-Matured Humanized Anti-PD-1 Antibodies

To improve anti-tumor efficacy at the low dose of the drug, affinity maturation was performed to generate multiple variants of affinity matured antibodies. Based on the previous anti-tumor efficacy data, the zCUSB0136.8 was used as a template and four affinity-matured antibodies were successfully generated including, zCUSB0136.8-1, zCUSB0136.8-2, zCUSB0136.8-3, and zCUSB0136.8-4. The binding affinity of affinity-matured antibodies are shown in Table 6. Based on the binding kinetic data, the zCUSB0136.8-2 was selected to confirm its in vivo anti-tumor efficacy at a low (2.5 mg/kg) and high (10 mg/kg) dose and compared to commercial antibodies.

An MC38 allograft/hPD-1 humanized mouse model was used for in vivo anti-tumor efficacy as described in Example 12. zCUSB0136.8-2, Keytruda®, and Opdivo® were used as treatment agents, while human IgG1 isotype control was used as a negative control. All treatments were performed by intraperitoneal injection every 4 days for 12 days.

Tumor volume measurement, routine monitoring, and any clinical signs were observed and recorded as described in Example 12.

### Efficacy Evaluation Criteria

The relative tumor growth inhibition rate (TGI) was calculated as follows: TGI %=(1−T/C)×100%, where T is tumor volume or tumor weight of treatment group and C is tumor volume or tumor weight of control group.

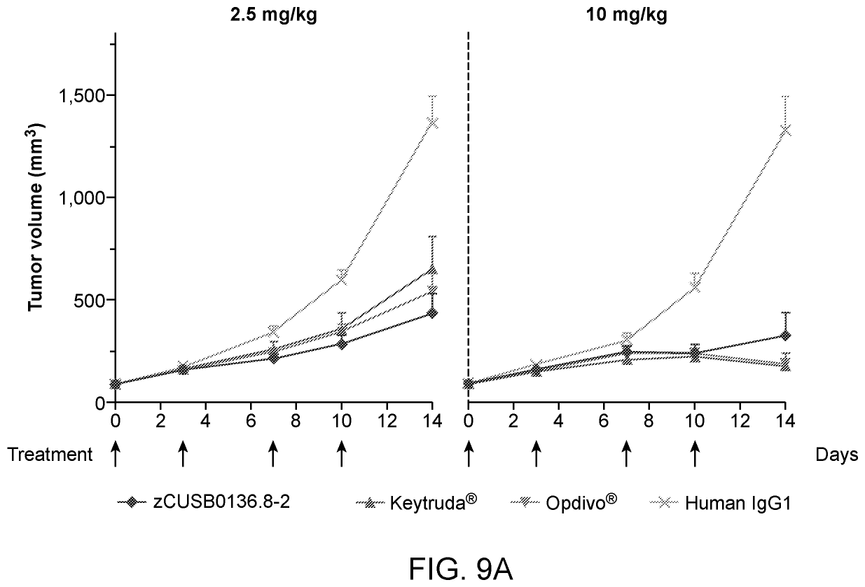

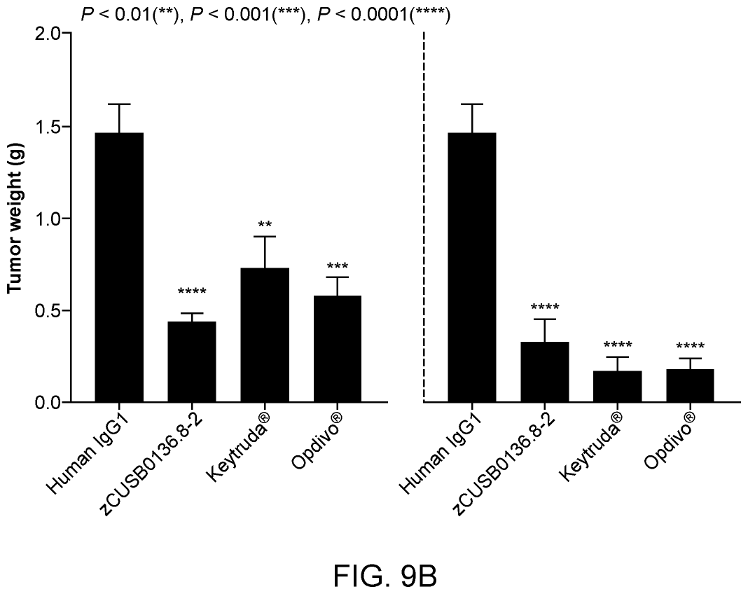

To evaluate anti-tumor efficacy of zCUSB0136.8-2 anti-PD-1 antibody, MC38 allograft/hPD-1 humanized mice (n=10 per group) were treated with 2.5 and 10 mg/kg every 4 days (i.e., on days 0, 3, 7, and 10). The tumor volume was monitored on days 0, 3, 7, 10, and 14. All mice were sacrificed on day 14 to measure the tumor weight. Tumor volume and tumor weight monitoring data are illustrated in FIG. 9. The TGI % was calculated as presented in Table 10.

TABLE 10
Anti-tumor efficacy of affinity-matured anti-PD-1 monoclonal antibodies
compared with commercial antibodies at the end of the study (day 14)

| | At dose 2.5 mg/kg | | | | At dose 10 mg/kg | | | |
|---|---|---|---|---|---|---|---|---|
| | Tumor | TGI % | Tumor | TGI % | Tumor | TGI % | Tumor | TGI % |
| | weight | (by | volume | (by | weight | (by | volume | (by |
| Antibody | (g) | weight) | (mm³) | volume) | (g) | weight) | (mm³) | volume) |
| Human IgG1 | 1.5 ± | — | 1363 ± | — | 1.5 ± | — | 1325 ± | — |
| | 0.2 | | 130 | | 0.2 | | 166 | |
| zCUSB0136.8- | 0.4 ± | 72.68 | 438 ± | 72.68 | 0.3 ± | 80.97 | 325 ± | 80.97 |
| 2-LALA | 0.1 | | 94 | | 0.1 | | 111 | |
| Keytruda ® | 0.7 ± | 55.83 | 652 ± | 55.83 | 0.2 ± | 92.82 | 178 ± | 92.82 |
| | 0.2 | | 156 | | 0.1 | | 58 | |
| Opdivo ® | 0.6 ± | 64.47 | 542 ± | 64.47 | 0.2 ± | 92.04 | 188 ± | 92.04 |
| | 0.1 | | 86 | | 0.1 | | 53 | |

Data are showed in Mean ± SEM

In summary, zCUSB0136.8-2-LALA exhibited high anti-tumor efficacy comparable to Keytruda® and Opdivo® in MC38 allograft/hPD-1 humanized mice.

### Example 14: X-Ray Crystallographic Analysis Between zCUSB0136.8-2 Fab and Human PD-1 Protein

### Crystallization

Crystals were obtained from sitting drops of 100 nL purified PD-1:zCUSB0136.8-2 Fab complex at 10 mg/mL and 100 nL of precipitant solution, consisting of 0.1 M bicine pH 9.3 and 22% v/v PEG Smear Broad. The crystallization drops were set up using a mosquito crystallization robot (STP Labtech) and were left to equilibrate against 40 μL precipitant solution at 20° C. The crystals were cryo-protected in precipitant solution supplemented with 20% v/v ethylene glycol prior to flash-cooling in liquid nitrogen

### Data Collection

The best data set was collected at beamline 103, Diamond Light Source, Didcot, UK, at 100 K and λ=0.9763 Å. 3,600 images were collected with an oscillation range of 0.10° per image. The beamline was equipped with an Eiger2 XE 16M detector. The data were processed anisotropically to 2.47 Å resolution using autoPROC (see Vonrhein et al., Acta Crystallogr. D Biol. Crystallogr. 67, 293-302 (2011)) and STARANISO (see Tickle et al. (2018) “STARANISO”, Cambridge, United Kingdom: Global Phasing Ltd.), which includes the software XDS (see Kabsch et al., Acta Crystallogr. D Biol. Crystallogr. 66, 125-132 (2010)), Aimless (see Evans et al., Acta. Crystallogr. D Biol. Crystallogr. 69, 1204-1214 (2013)), and Pointless (see Evans et al., Acta. Crystallogr. D Biol. Crystallogr. 62, 72-82(2006)). Statistics for a dataset processed isotropically to 2.7 Å resolution are provided for comparison, though this dataset was not used to build and refine the model.

### Structure Determination and Refinement

Initial phases were obtained by molecular replacement using Phaser (see McCoy et al., J. Appl. Crystallogr. 40, 658-674 (2007)) and a published structure of a PD1: zCUSB0136.8-2 Fab complex (PDB entry: 7BXA) where the PD-1 and Fab molecule were split into two separate search ensembles. The structure refinement was carried out using Buster (see Bricogne et al., (2017) BUSTER 2.11.8, Cambridge, United Kingdom: Global Phasing Ltd.) and model building was carried out in Coot (see Emsley et al., Acta Crystallogr. D Biol. Crystallogr. 66, 486-501 (2010)). The final R-values (R_{model}/R_{free}) were 0.204/0.239.

The Structure of the PD-1:zCUSB0136.8-2 Fab Complex

The structure of the PD-1:zCUSB0136.8-2 Fab complex was determined to 2.47 Å resolution with two PD-1: zCUSB0136.8-2 Fab complexes in the asymmetric unit. The first PD-1:zCUSB0136.8-2 Fab complex includes heavy chain residues 1-126 and 128-212 (chain A), light chain residues 1-216 (chain B), and PD-1 residues 33-88 and 93-145 (chain C). The second PD1:zCUSB0136.8-2 Fab complex includes heavy chain residues 1-127 and 133-212 (chain H), light chain residues 1-217 (chain L), and PD-1 residues 33-84 and 92-147 (chain P). The structure is overall well-defined in the electron density maps. However, some residues of PD-1 could not be built due to weak electron density, including residues 86-88 in chain P that are visible in chain C and located near the binding interface with the CDR2 loop of the light chain. The epitope interactions were calculated using CONTACT and CCP4. An epitope-paratope analysis is summarized in Table 11.

TABLE 11
Lists of epitope contacts with a strong possibility
of a hydrogen bond (distance < 3.3 Å) or
salt bridge between zCUSB0136.8-2 Fab and human PD-1 protein

| PD-1 Residue | Fab Residue | Fab Domain | Bond Type |
|---|---|---|---|
| Glu61 | Arg97 | VH-CDR3 | Salt bridge |
| Glu61 | Tyr101 | VH-CDR3 | H-bond |
| Ser62 | Gln59 | VL-CDR2 | H-bond |
| Phe63 | Tyr53 | VL-CDR2 | H-bond |
| Asn66 | Asn34 | VL-CDR1 | H-bond |
| Tyr68 | Tyr32 | VL-CDR1 | H-bond |
| Lys78 | Gly33 | VL-CDR1 | H-bond |
| Arg86 | Ser56 | VL-CDR2 | H-bond |
| Arg86 | Phe66 | VL-FR2 | H-bond |
| Arg86 | Asp64 | VL-FR2 | H-bond |
| Gln88 | Gly70 | VL-FR2 | H-bond |
| Gln88 | Gly68 | VL-FR2 | H-bond |
| Ala129 | Asn95 | VL-CDR3 | H-bond |
| Pro130 | Trp99 | VH-CDR3 | H-bond |
| Lys131 | Asn96 | VL-CDR3 | H-bond |
| Ala132 | Asn95 | VL-CDR3 | H-bond |

In conclusion, the structure of the PD-1:zCUSB0136.8-2 Fab complex was successfully evaluated and the epitope contact residues between the complex were identified.

### Example 15: PD-1/PD-L2 Ligand Binding Blockage of Affinity-Matured Antibody

The PD-1/PD-L2 ligand binding blockage of the zCUSB0136.8-2 was evaluated using flow cytometry. Initially, 4-fold serial dilutions of the zCUSB0136.8-2 antibody starting at 2 μg/mL were mixed with recombinant human PD-L2 protein mouse Fe Tag (Sino Biological) at 1.25 μg/mL. Subsequently, this mixture was added to PD-1-expressing Jurkat effector cells (5×10⁴ cells/well) in FPBS buffer, and mixed by gently pipetted.

The plate was incubated at room temperature for 30 min and then centrifuged to harvest the cells (500 g, at room temperature for 5 min), and the supernatant was discarded. To detect either PD-L2 protein mouse Fc Tag or affinity-matured zCUSB0136.8-2 human IgG1-LALA antibody that bound to the hPD-1-expressing Jurkat cells, a secondary antibody mixture (Alexa Fluor 488 goat anti-mouse IgG (Jackson ImmunoResearch) and Alexa Fluor 647 goat anti-human IgG (Jackson ImmunoResearch) mixed at 1:250 in FPBS buffer) was added to each well (100 μL) and then gently mixed by pipetting. The plate was incubated in the dark at room temperature for 30 min and washed twice with FPBS buffer. Cells were resuspended in 100 μL FPBS buffer. The Intellicyt iQue Screener Plus flow cytometer was used to detect the fluorescent signal, and data were analyzed with Forecyt® Standard Edition 7.1 software. The half-maximal inhibitory concentration (IC₅₀) was calculated using GraphPad Prism® software

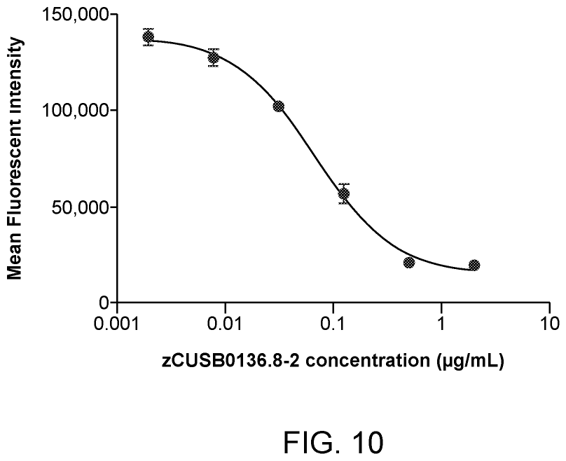

The result shows that affinity-matured zCUSB0136.8-2 antibody can inhibit PD-1/PD-L2 interaction with an IC₅₀ value of 66.76 ng/mL (FIG. 10).

In conclusion, the zCUSB0136.8-2-LALA showed high PD-1/PD-L2 binding blockade activity assessed by flow-cytometry.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

The foregoing description has been presented only for the purposes of illustration and is not intended to limit the disclosure to the precise form disclosed, but by the claims appended hereto.

1. An anti-PD-1 antibody or antigen-binding fragment thereof comprising:
(a) (i) a VH comprising a CDR1 comprising the sequence X₁YDII (SEQ ID NO: 228), wherein X₁ is T or V, a CDR2 comprising the sequence VIWTGGDTX₂YNSAFMS (SEQ ID NO: 229), wherein X₂ is N, W or T, and a CDR3 comprising the sequence DWX₃Y (SEQ ID NO: 230), wherein X₃ is A, I or M; and (ii) a VL comprising a CDR1 comprising the sequence RASESVDNX₄GNSFIQ (SEQ ID NO: 231), wherein X₄ is Y or W, a CDR2 comprising the sequence FASX₅LQS (SEQ ID NO: 232), wherein X₅ is N, Y, H or Q, and a CDR3 comprising the sequence HQNNEDPFT (SEQ ID NO: 227);
(b) (i) a VH comprising a CDR1 comprising the sequence set forth in SEQ ID NO: 162, a CDR2 comprising the sequence set forth in SEQ ID NO: 163, and a CDR3 comprising the sequence set forth in SEQ ID NO: 164; and (ii) comprising a VL comprising a CDR1 comprising the sequence set forth in SEQ ID NO: 180, a CDR2 comprising the sequence set forth in SEQ ID NO: 181, and a CDR3 comprising the sequence set forth in SEQ ID NO: 182;
(c) (i) a VH comprising a CDR1 comprising the sequence set forth in SEQ ID NO: 81, a CDR2 comprising the sequence set forth in SEQ ID NO: 82, and a CDR3 comprising the sequence set forth in SEQ ID NO: 83; and (ii) comprising a VL comprising a CDR1 comprising the sequence set forth in SEQ ID NO: 85, a CDR2 comprising the sequence set forth in SEQ ID NO: 86, and a CDR3 comprising the sequence set forth in SEQ ID NO: 87;
(d) (i) a VH comprising a CDR1 comprising the sequence set forth in SEQ ID NO: 89, a CDR2 comprising the sequence set forth in SEQ ID NO: 90, and a CDR3 comprising the sequence set forth in SEQ ID NO: 91; and (ii) comprising a VL comprising a CDR1 comprising the sequence set forth in SEQ ID NO: 93, a CDR2 comprising the sequence set forth in SEQ ID NO: 94, and a CDR3 comprising the sequence set forth in SEQ ID NO: 95;
(e) (i) a VH comprising a CDR1 comprising the sequence set forth in SEQ ID NO: 97, a CDR2 comprising the sequence set forth in SEQ ID NO: 98, and a CDR3 comprising the sequence set forth in SEQ ID NO: 99; and (ii) comprising a VL comprising a CDR1 comprising the sequence set forth in SEQ ID NO: 101, a CDR2 comprising the sequence set forth in SEQ ID NO: 102, and a CDR3 comprising the sequence set forth in SEQ ID NO: 103;
(f) (i) a VH comprising a CDR1 comprising the sequence set forth in SEQ ID NO: 113, a CDR2 comprising the sequence set forth in SEQ ID NO: 114, and a CDR3 comprising the sequence set forth in SEQ ID NO: 115; and (ii) comprising a VL comprising a CDR1 comprising the sequence set forth in SEQ ID NO: 117, a CDR2 comprising the sequence set forth in SEQ ID NO: 118, and a CDR3 comprising the sequence set forth in SEQ ID NO: 119; or
(g) (i) a VH comprising a CDR1 comprising the sequence set forth in SEQ ID NO: 121, a CDR2 comprising the sequence set forth in SEQ ID NO: 122, and a CDR3 comprising the sequence set forth in SEQ ID NO: 123; and (ii) comprising a VL comprising a CDR1 comprising the sequence set forth in SEQ ID NO: 125, a CDR2 comprising the sequence set forth in SEQ ID NO: 126, and a CDR3 comprising the sequence set forth in SEQ ID NO: 127.

2. The anti-PD-1 antibody or antigen-binding fragment thereof of claim 1 comprising:
(i) a VH comprising a CDR1 comprising the sequence set forth in SEQ ID NO: 129, a CDR2 comprising the sequence set forth in SEQ ID NO: 130, and a CDR3 comprising the sequence set forth in SEQ ID NO: 131; and (ii) comprising a VL comprising a CDR1 comprising the sequence set forth in SEQ ID NO: 133, a CDR2 comprising the sequence set forth in SEQ ID NO: 134, and a CDR3 comprising the sequence set forth in SEQ ID NO: 135.

3. The anti-PD-1 antibody or antigen-binding fragment thereof of claim 1 comprising:
(a) a VH comprising the sequence X₆VQLX₇ESGPGLVKPSQX₈LSLTCTVX₉GYSITSDYAWNWIRQX₁₀PGX₁₁X₁₂LEW X₁₃GYIYYSGTTSYNPSLKSRX₁₄X₁₅IX₁₆X₁₇DTSKNQFX₁₈LX₁₉LX₂₀SVTX₂₁X₂₂DTA X₂₃YYCARNYGSAFYYFDYWGQGTTLTVSS, wherein X₆ is D or Q; X₇ is R or Q; X₈ is S or T; X₉ is T or S; X₁₀ is F or P; X₁₁ is N or K; X₁₂ is K or G; X₁₃ is M or I; X₁₄ is I or V; X₁₅ is S or T; X₁₆ is T or S; X₁₇ is R or V; X₁₈ is F or S; X₁₉ is Q or K; X₂₀ is N or S; X₂₁ is T or A; X₂₂ is E or A; and/or X₂₃ is T or V; or
(b) a VH comprising the sequence QVQLX₃₉ESGPGLVX₄₀PSX₄₁X₄₂LSX₄₃TCTVSGFSLTX₄₄YDIIWIRQX₄₅PGKGLEWX₄₆GVIWTGGDTX₄₇YNSAFMSRX₄₈X₄₉ISX₅₀DX₅₁SKX₅₂QX₅₃X₅₄LKX₅₅X₅₆SX₅₇X₅₈X₅₉X₆₀DTAX₆₁YYCX₆₂RDWX₆₃YWGQGTLVTVSX₆₄, wherein X₃₉ is K or Q; X₄₀ is A or K; X₄₁ is Q or E; X₄₂ is S or T; X₄₃ is I or L; X₄₄ is T or V; X₄₅ is S or P; X₄₆ is L or I; X₄₇ is N, W or T; X₄₈ is L or V; X₄₉ is T or S; X₅₀ is K or V; X₅₁ is N or T; X₅₂ is S or N; X₅₃ is I or F; X₅₄ is F or S; X₅₅ is M or L; X₅₆ is N or S; X₅₇ is L or V; X₅₈ is Q or T; X₅₉ is T or A; X₆₀ is D or A; X₆₁ is I or V; X₆₂ is A or V; X₆₃ is A, I or M; and/or X₆₄ is T or S.

4. The anti-PD-1 antibody or antigen-binding fragment thereof of claim 1 comprising:
(a) a VL comprising the sequence DIX₂₄MTQSPSSX₂₅SX₂₆SX₂₇GDX₂₈VTITCHASQGISSNIGWX₂₉QQKPGKX₃₀X₃₁KX₃₂LIYRGTNLEDGVPSRFSGSGSGX₃₃DX₃₄X₃₅LTISSLX₃₆X₃₇EDFAX₃₈YYCVQYAQF PPTFGGGTKLEIK, wherein X₂₄ is L or Q; X₂₅ is M or V; X₂₆ is V or A; X₂₇ is L or V; X₂₈ is T or R; X₂₉ is L or Y; X₃₀ is S or A; X₃₁ is F or P; X₃₂ is G or L; X₃₃ is A or T; X₃₄ is Y or F; X₃₅ is S or T; X₃₆ is E or Q; X₃₇ is S or P; and/or X₃₈ is D or T; or
(b) a VL comprising the sequence X₆₅IVX₆₆TQSPX₆₇SLAVSLGX₆₈RATIX₆₉CRASESVDNX₇₀GNSFIQWYQQKPGQPPK LLIYFASX₇₁LQSGVPXnRFSGSGSX₇₃TDFTLTIX₇₄X₇₅X₇₆X₇₇AX₇₈DX₇₉AX₈₀YYCH QNNEDPFTFGX₈₁GTKLEIK, wherein X₆₅ is N or D; X₆₆ is L or M; X₆₇ is A or D; X₆₈ is Q or E; X₆₉ is S or N; X₇₀ is Y or W; X₇₁ is N or Y; X₇₂ is A or D; X₇₃ is R or G; X₇₄ is D or S; X₇₅ is P or S; X₇₆ is V or L; X₇₇ is E or Q; X₇₈ is D or E; X₇₉ is A or V; X₈₀ is T or V; and/or X₈₁ is S or G.

5. The anti-PD-1 antibody or antigen-binding fragment thereof of claim 1 comprising a VH comprising a sequence that is at least 80%, at least 85%, at least 90%, at least 95%, at least 96%, at least 97%, at least 98%, or at least 99% identical to the sequence set forth in any one of SEQ ID NOs: 80, 88, 96, 104, 112, 120, 128, 136, 137, 138, 139, 140, 144, 145, 146, 147, 148, 149, 153, 154, 155, and 156.

6. The anti-PD-1 antibody or antigen-binding fragment thereof of claim 1 comprising a VL comprising a sequence that is at least 80%, at least 85%, at least 90%, at least 95%, at least 96%, at least 97%, at least 98%, or at least 99% identical to the sequence set forth in any one of SEQ ID NOs: 84, 92, 100, 108, 116, 124, 132, 141, 142, 143, 150, 151, 152, 157, and 158.

7. The anti-PD-1 antibody or antigen-binding fragment thereof of claim 1 comprising a VH comprising the sequence set forth in any one of SEQ ID NOs: 80, 88, 96, 104, 112, 120, 128, 136, 137, 138, 139, 140, 144, 145, 146, 147, 148, 149, 153, 154, 155, and 156.

8. The anti-PD-1 antibody or antigen-binding fragment thereof of claim 1, comprising a VL comprising the sequence set forth in any one of SEQ ID NOs: 84, 92, 100, 108, 116, 124, 132, 141, 142, 143, 150, 151, 152, 157, and 158.

9. The anti-PD-1 antibody or antigen-binding fragment thereof of claim 1, comprising:
(a) a VH comprising the sequence set forth in SEQ ID NO: 136 and a VL comprising the sequence set forth in SEQ ID NO: 143;
(b) a VH comprising the sequence set forth in SEQ ID NO: 137 and a VL comprising the sequence set forth in SEQ ID NO: 143;
(c) a VH comprising the sequence set forth in SEQ ID NO: 138 and a VL comprising the sequence set forth in SEQ ID NO: 143;
(d) a VH comprising the sequence set forth in SEQ ID NO: 139 and a VL comprising the sequence set forth in SEQ ID NO: 143;
(e) a VH comprising the sequence set forth in SEQ ID NO: 145 and a VL comprising the sequence set forth in SEQ ID NO: 150;
(f) a VH comprising the sequence set forth in SEQ ID NO: 145 and a VL comprising the sequence set forth in SEQ ID NO: 151;
(g) a VH comprising the sequence set forth in SEQ ID NO: 145 and a VL comprising the sequence set forth in SEQ ID NO: 152;
(h) a VH comprising the sequence set forth in SEQ ID NO: 146 and a VL comprising the sequence set forth in SEQ ID NO: 150;
(i) a VH comprising the sequence set forth in SEQ ID NO: 146 and a VL comprising the sequence set forth in SEQ ID NO: 151;
(j) a VH comprising the sequence set forth in SEQ ID NO: 146 and a VL comprising the sequence set forth in SEQ ID NO: 152;
(k) a VH comprising the sequence set forth in SEQ ID NO: 153 and a VL comprising the sequence set forth in SEQ ID NO: 157;
(l) a VH comprising the sequence set forth in SEQ ID NO: 154 and a VL comprising the sequence set forth in SEQ ID NO: 158;
(m) a VH comprising the sequence set forth in SEQ ID NO: 155 and a VL comprising the sequence set forth in SEQ ID NO: 158;
(n) a VH comprising the sequence set forth in SEQ ID NO: 156 and a VL comprising the sequence set forth in SEQ ID NO: 158;
(o) a VH comprising the sequence X₆VQLX₇ESGPGLVKPSQX₈LSLTCTVX₉GYSITSDYAWNWIRQX₁₀PGX₁₁X₁₂LEW X₁₃GYIYYSGTTSYNPSLKSRX₁₄X₁₅IX₁₆X₁₇DTSKNQFX₁₈LX₁₉LX₂₀SVTX₂₁X₂₂DTA X₂₃YYCARNYGSAFYYFDYWGQGTTLTVSS, wherein X₆ is D or Q; X₇ is R or Q; X₈ is S or T; X₉ is T or S; X₁₀ is F or P; X₁₁ is N or K; X₁₂ is K or G; X₁₃ is M or I; X₁₄ is I or V; X₁₅ is S or T; X₁₆ is T or S; X₁₇ is R or V; X₁₈ is F or S; X₁₉ is Q or K; X₂₀ is N or S; X₂₁ is T or A; X₂₂ is E or A; and X₂₃ is T or V; and a VL comprising the sequence DIX₂₄MTQSPSSX₂₅SX₂₆SX₂₇GDX₂₈VTITCHASQGISSNIGWX₂₉QQKPGKX₃₀X₃₁KX₃₂LIYRGTNLEDGVPSRFSGSGSGX₃₃DX₃₄X₃₅LTISSLX₃₆X₃₇EDFAX₃₈YYCVQYAQF PPTFGGGTKLEIK, wherein X₂₄ is L or Q; X₂₅ is M or V; X₂₆ is V or A; X₂₇ is L or V; X₂₈ is T or R; X₂₉ is L or Y; X₃₀ is S or A; X₃₁ is F or P; X₃₂ is G or L; X₃₃ is A or T; X₃₄ is Y or F; X₃₅ is S or T; X₃₆ is E or Q; X₃₇ is S or P; and X₃₈ is D or T; or
(p) a VH comprising the sequence QVQLX₃₉ESGPGLVX₄₀PSX₄₁X₄₂LSX₄₃TCTVSGFSLTX₄₄YDIIWIRQX₄₅PGKGLEWX₄₆GVIWTGGDTX₄₇YNSAFMSRX₄₈X₄₉ISX₅₀DX₅₁SKX₅₂QX₅₃X₅₄LKX₅₅X₅₆SX₅₇X₅₈X₅₉X₆₀DTAX₆₁YYCX₆₂RDWX₆₃YWGQGTLVTVSX₆₄, wherein X₃₉ is K or Q; X₄₀ is A or K; X₄₁ is Q or E; X₄₂ is S or T; X₄₃ is I or L; X₄₄ is T or V; X₄₅ is S or P; X₄₆ is L or I; X₄₇ is N, W or T; X₄₈ is L or V; X₄₉ is T or S; X₅₀ is K or V; X₅₁ is N or T; X₅₂ is S or N; X₅₃ is I or F; X₅₄ is F or S; X₅₅ is M or L; X₅₆ is N or S; X₅₇ is L or V; X₅₈ is Q or T; X₅₉ is T or A; X₆₀ is D or A; X₆₁ is I or V; X₆₂ is A or V; X₆₃ is A, I or M; and X₆₄ is T or S; and a VL comprising the sequence X₆₅IVX₆₆TQSPX₆₇SLAVSLGX₆₈RATIX₆₉CRASESVDNX₇₀GNSFIQWYQQKPGQPPK LLIYFASX₇₁LQSGVPX₇₂RFSGSGSX₇₃TDFTLTIX₇₄X₇₅X₇₆X₇₇AX₇₈DX₇₉AX₈₀YYCH QNNEDPFTFGX₈₁GTKLEIK, wherein X₆₅ is N or D; X₆₆ is L or M; X₆₇ is A or D; X₆₈ is Q or E; X₆₉ is S or N; X₇₀ is Y or W; X₇₁ is N or Y; X₇₂ is A or D; X₇₃ is R or G; X₇₄ is D or S; X₇₅ is P or S; X₇₆ is V or L; X₇₇ is E or Q; X₇₈ is D or E; X₇₉ is A or V; X₈₀ is T or V; and X₈₁ is S or G.

10. The anti-PD-1 antibody or antigen-binding fragment thereof of claim 1, wherein the antibody is a monoclonal antibody.

11. The anti-PD-1 antibody or antigen-binding fragment thereof of claim 1, wherein the antibody or antigen-binding fragment thereof is a humanized antibody or antigen-binding fragment thereof.

12. The anti-PD-1 antibody or antigen-binding fragment thereof of claim 1, wherein the antibody or antigen-binding fragment thereof is an IgG1 antibody or antigen-binding fragment thereof.

13. The anti-PD-1 antibody or antigen-binding fragment thereof of claim 1, wherein the antigen-binding fragment is a Fab fragment, Fab′ fragment, F(ab′)2 fragment, scFv, dsFv, ds-scFv, or a nanobody.

14. The anti-PD-1 antibody or antigen-binding fragment thereof of claim 1, wherein the antibody has a L234AL235A mutation in Fc region.

15. The anti-PD-1 antibody or antigen-binding fragment thereof of claim 1 wherein the antibody or antigen-binding fragment thereof binds to PD-1 with a K_{D} of less than 0.5 nM, less than 0.3 nM, or less than 0.1 nM, less than 0.05 nM, less than 0.02 nM.

16. The anti-PD-1 antibody or antigen-binding fragment thereof of claim 1, wherein the antibody or antigen-binding fragment inhibits the interaction between PD-1 and PD-L1 with an IC₅₀ of less than about 500 ng/mL, less than about 400 ng/mL, or less than about 300 ng/mL.

17. The anti-PD-1 antibody or antigen-binding fragment thereof of claim 1, wherein the antibody or antigen-binding fragment activates T cells.

18. The anti-PD-1 antibody or antigen-binding fragment thereof of claim 1, wherein the antibody or antigen-binding fragment induces IL-2 secretion in T cells.

19. The anti-PD-1 antibody or antigen-binding fragment thereof of claim 1, wherein the antibody or antigen-binding fragment thereof induces IFN-γ secretion in T cells.

20. A pharmaceutical composition comprising the anti-PD-1 antibody or antigen-binding fragment thereof and a pharmaceutically acceptable carrier.

21. A method of treating cancer in a subject, comprising administering to the subject the anti-PD-1 antibody or antigen-binding fragment thereof of claim 1 or the pharmaceutical composition of claim 20.

22. The method of claim 21, wherein the cancer is colon cancer, breast cancer, lung cancer, ovarian cancer, prostate cancer, cervical cancer, brain cancer, skin cancer, liver cancer, pancreatic cancer or stomach cancer.

23. The method of claim 21, wherein the cancer is a leukemia or a lymphoma.

24. A method of treating an infectious disease in a subject, comprising administering to the subject the anti-PD-1 antibody or antigen-binding fragment thereof of claim 1 or the pharmaceutical composition of claim 20.

25. The method of claim 24, wherein the infectious disease is tuberculosis, malaria, an HIV infection, or a HBV infection.

26. A method of treating primary immunodeficiency in a subject, comprising administering to the subject the anti-PD-1 antibody or antigen-binding fragment thereof of claim 1 or the pharmaceutical composition of claim 20.

27. The anti-PD-1 antibody or antigen-binding fragment thereof of claim 1 or the pharmaceutical composition of claim 20 for the treatment of cancer.

28. The anti-PD-1 antibody or antigen-binding fragment thereof of claim 27, wherein the cancer is colon cancer, breast cancer, lung cancer, ovarian cancer, prostate cancer, cervical cancer, brain cancer, skin cancer, liver cancer, pancreatic cancer or stomach cancer.

29. The anti-PD-1 antibody or antigen-binding fragment thereof of claim 27, wherein the cancer is a leukemia or a lymphoma.

30. The anti-PD-1 antibody or antigen-binding fragment thereof of claim 1 or the pharmaceutical composition of claim 20 for the treatment of an infectious disease.

31. The anti-PD-1 antibody or antigen-binding fragment thereof of claim 30, wherein the infectious disease is tuberculosis, malaria, an HIV infection, or a HBV infection.

32. The anti-PD-1 antibody or antigen-binding fragment thereof of claim 1 or the pharmaceutical composition of claim 20 for the treatment of primary immunodeficiency.

33. Use of the anti-PD-1 antibody or antigen-binding fragment thereof of claim 1 in the manufacture of a medicament for the treatment of cancer.

34. The use of claim 33, wherein the cancer is colon cancer, breast cancer, lung cancer, ovarian cancer, prostate cancer, cervical cancer, brain cancer, skin cancer, liver cancer, pancreatic cancer or stomach cancer.

35. The use of claim 33, wherein the cancer is a leukemia or a lymphoma.

36. Use of the anti-PD-1 antibody or antigen-binding fragment thereof of claim 1 in the manufacture of a medicament for the treatment of an infectious disease.

37. The use of claim 36, wherein the infectious disease is tuberculosis, malaria, an HIV infection, or a HBV infection.

38. Use of the anti-PD-1 antibody or antigen-binding fragment thereof of claim 1 in the manufacture of a medicament for the treatment of primary immunodeficiency.

39. A method of inhibiting the interaction between PD-1 and PD-L1 in a cell, comprising contacting the cell with an antibody or antigen-binding fragment thereof of claim 1.

40. A method of inhibiting the interaction between PD-1 and PD-L2 in a cell, comprising contacting the cell with an antibody or antigen-binding fragment thereof of claim 1.

41. A method of stimulating IL-2 secretion in a cell, comprising contacting the cell with an antibody or antigen-binding fragment thereof of claim 1.

42. A method of stimulating IFN-γ secretion in a cell, comprising contacting the cell with an antibody or antigen-binding fragment thereof of claim 1.

43. The method of claim 39, wherein the cell is a human cell.

44. The method of claim 39, wherein the cell is contacted in vitro.

45. The method of claim 39, wherein the cell is contacted in vivo.


SEQUENCE LISTING (236 sequences)

SEQ ID NO: 1 - DNA, 408 bp, synthetic construct
  misc_feature 1..408: note = CUSB0103-VH
  source 1..408: mol_type = other DNA
atgaacttcgggctcagattgattttccttgtccttactttaaaaggtgtccagtgtgacgtgaagctggtggagtctgggggaggcttggtgaagcctggagggtccctgaaactctcctgtgcagcctctggattcactttcagtagctataccatgtcttgggttcgccagactccggagaagaggctggagtgggtcgcagccattagtggtggtggtagttacacctactatgcagacagtgttaagggccgattcaccatctccagagacaatgccaagaacaccctgtccctgcaaatgagcagtctgaagtctgaggacacagccatgtatttctgtacaagtccctatgatttcgctatggactactggggtcaaggaacctcagtcaccgtctcttca

SEQ ID NO: 2 - DNA, 15 bp, synthetic construct
  misc_feature 1..15: note = CUSB0103-VH-CDR1
  source 1..15: mol_type = other DNA
agctataccatgtct

SEQ ID NO: 3 - DNA, 51 bp, synthetic construct
  misc_feature 1..51: note = CUSB0103-VH-CDR2
  source 1..51: mol_type = other DNA
gccattagtggtggtggtagttacacctactatgcagacagtgttaagggc

SEQ ID NO: 4 - DNA, 24 bp, synthetic construct
  misc_feature 1..24: note = CUSB0103-VH-CDR3
  source 1..24: mol_type = other DNA
ccctatgatttcgctatggactac

SEQ ID NO: 5 - DNA, 378 bp, synthetic construct
  misc_feature 1..378: note = CUSB0103-VL
  source 1..378: mol_type = other DNA
atgtcctctgctcagttccttggtctcctgttgctctgttttcaaggtaccagatgtgatatccagatgacacagactacatcctccctgtctgcctctctgggagacagagtcaccatcagttgcagggcaagtcaggacattaccaattatttaaactggtatcaacagaaaccagatggatctgttaaactcctgatctactacacatcaagattacactcaggagtcccatcaaggttccgtggcagtgggtctggaacagattattctctcaccattagtaacctggagcaagaagatattgccacttacttttgccaacagggtaatacgcttccgtggacgttcggtggaggcaccaagctggaaatcaaa

SEQ ID NO: 6 - DNA, 33 bp, synthetic construct
  misc_feature 1..33: note = CUSB0103-VL-CDR1
  source 1..33: mol_type = other DNA
agggcaagtcaggacattaccaattatttaaac

SEQ ID NO: 7 - DNA, 21 bp, synthetic construct
  misc_feature 1..21: note = CUSB0103-VL-CDR2
  source 1..21: mol_type = other DNA
tacacatcaagattacactca

SEQ ID NO: 8 - DNA, 27 bp, synthetic construct
  misc_feature 1..27: note = CUSB0103-VL-CDR3
  source 1..27: mol_type = other DNA
caacagggtaatacgcttccgtggacg

SEQ ID NO: 9 - DNA, 417 bp, synthetic construct
  misc_feature 1..417: note = CUSB0123-VH
  source 1..417: mol_type = other DNA
atgggttggagctgtatcatcttctttctggtagcaacagctacaggtgtgcactcccaggtccagctacaacagtctgggcctgagctggtgaggcctggggaatcagtgaagatttcctgtaagggttccggctacacattcactgattatgttatgcactgggtgaagcagagtcatgcaaagagtctagagtggattggagttattactatttacaatgatattacaaactacaaccagaaatttaagggcaaggccacaatgactatagacaaatcctccagcacagcctatatggaacttgccagattgacatctgaggattctgccatctattactgtgcaagagaggggtatgattcctactggtacttcgatgtctggggcgcagggaccacggtcaccgtctcctca

SEQ ID NO: 10 - DNA, 15 bp, synthetic construct
  misc_feature 1..15: note = CUSB0123-VH-CDR1
  source 1..15: mol_type = other DNA
gattatgttatgcac

SEQ ID NO: 11 - DNA, 51 bp, synthetic construct
  misc_feature 1..51: note = CUSB0123-VH-CDR2
  source 1..51: mol_type = other DNA
gttattactatttacaatgatattacaaactacaaccagaaatttaagggc

SEQ ID NO: 12 - DNA, 33 bp, synthetic construct
  misc_feature 1..33: note = CUSB0123-VH-CDR3
  source 1..33: mol_type = other DNA
gaggggtatgattcctactggtacttcgatgtc

SEQ ID NO: 13 - DNA, 393 bp, synthetic construct
  misc_feature 1..393: note = CUSB0123-VL
  source 1..393: mol_type = other DNA
atgaagttgcctgttaggctgttggtgctgatgttctggattcctgcttccagcagtgatgttttgatgacccaaactccactctccctgcctgtcagtcttggagatcaagcctccatctcttgcagatctagtcaaaccattgtacatagtaatggaaacacctatttagaatggtacctgcagaaaccaggccagtctccaaagctcctgatctacaaagtttccaaccgattttctggggtcccagacaggttcagtggcagtggatcagggacagattttacactcaagatcagcagagtggaggctgaggatctgggagtttattactgctttcaaggttcacatcttccgtacacgttcggaggggggaccaagctggaaataaaa

SEQ ID NO: 14 - DNA, 48 bp, synthetic construct
  misc_feature 1..48: note = CUSB0123-VL-CDR1
  source 1..48: mol_type = other DNA
agatctagtcaaaccattgtacatagtaatggaaacacctatttagaa

SEQ ID NO: 15 - DNA, 21 bp, synthetic construct
  misc_feature 1..21: note = CUSB0123-VL-CDR2
  source 1..21: mol_type = other DNA
aaagtttccaaccgattttct

SEQ ID NO: 16 - DNA, 27 bp, synthetic construct
  misc_feature 1..27: note = CUSB0123-VL-CDR3
  source 1..27: mol_type = other DNA
tttcaaggttcacatcttccgtacacg

SEQ ID NO: 17 - DNA, 408 bp, synthetic construct
  misc_feature 1..407: note = CUSB0124-VH
  source 1..408: mol_type = other DNA
atggattggctgtggaacttgctattcctgatggcagctgcccaaagtatccaagcacagatccagttggtgcagtctggacctgagctgaagaagcctggagagacagtcaagatctcctgcaaggcttctgggtatatcttcacaaactatggagtgacctgggtgaagcagactccaggaaagggtttaaagtggatgacctggataaacaccaacactggagagccaacatctgctgaagagttcaagggacggtttgacttctcttcggaaacctctgccaccactgcctatttgcagatcaacaacctcaaaaatgaggacacggctacatatttctgtgtaagaatacgggagaatgctatggactactggggtcaaggaacctcagtcaccgtctcctca

SEQ ID NO: 18 - DNA, 15 bp, synthetic construct
  misc_feature 1..15: note = CUSB0124-VH-CDR1
  source 1..15: mol_type = other DNA
aactatggagtgacc

SEQ ID NO: 19 - DNA, 51 bp, synthetic construct
  misc_feature 1..51: note = CUSB0124-VH-CDR2
  source 1..51: mol_type = other DNA
tggataaacaccaacactggagagccaacatctgctgaagagttcaaggga

SEQ ID NO: 20 - DNA, 24 bp, synthetic construct
  misc_feature 1..24: note = CUSB0124-VH-CDR3
  source 1..24: mol_type = other DNA
atacgggagaatgctatggactac

SEQ ID NO: 21 - DNA, 381 bp, synthetic construct
  misc_feature 1..381: note = CUSB0124-VL
  source 1..381: mol_type = other DNA
atgaacatgctcactcagctcctgggattactgctgctctggtttgcaggtggtaaatgtgacattcagatgacccagtctcctgcctcccagtctgcatctctgggagaaagtgtcaccatcacatgcctggcaagtcagaccattggtacctggttggcatggtatcagcagaaaccagggaaatctcctcagctcctgatttatgctgcaaccaggttggcagttggggtcccatcaaggttcagtggtagtggatctggcacaaaattttctttcaagatcagcagactgcaggctgaagattttgtaagttattactgtcaacaactttacagtactccattcacgttcggctcggggacaaagttggagataaaa

SEQ ID NO: 22 - DNA, 33 bp, synthetic construct
  misc_feature 1..33: note = CUSB0124-VL-CDR1
  source 1..33: mol_type = other DNA
ctggcaagtcagaccattggtacctggttggca

SEQ ID NO: 23 - DNA, 21 bp, synthetic construct
  misc_feature 1..21: note = CUSB0124-VL-CDR2
  source 1..21: mol_type = other DNA
gctgcaaccaggttggcagtt

SEQ ID NO: 24 - DNA, 27 bp, synthetic construct
  misc_feature 1..27: note = CUSB0124-VL-CDR3
  source 1..27: mol_type = other DNA
caacaactttacagtactccattcacg

SEQ ID NO: 25 - DNA, 414 bp, synthetic construct
  misc_feature 1..414: note = CUSB0125-VH
  source 1..414: mol_type = other DNA
atgagagtgctgattcttttgtggctgttcacagcctttcctggtatcctgtctgatgtgcagcttcgggagtcgggacctggcctggtgaaaccttctcagtctctgtccctcacctgcactgtcactggctactcaatcaccagtgattatgcctggaactggatccggcagtttccaggaaacaaactggagtggatgggctacatatactacagtggtaccactagttacaacccatctctcaaaagtcgaatctctatcactcgagacacatccaagaaccagttcttcctgcagttgaattctgtgactactgaggacacagccacgtattactgtgcaagaaactacggtagtgccttttactactttgactactggggccaaggcaccactctcacagtctcctca

SEQ ID NO: 26 - DNA, 17 bp, synthetic construct
  misc_feature 1..17: note = CUSB0125-VH-CDR1
  source 1..17: mol_type = other DNA
gtgattatgcctggaac

SEQ ID NO: 27 - DNA, 48 bp, synthetic construct
  misc_feature 1..48: note = CUSB0125-VH-CDR2
  source 1..48: mol_type = other DNA
tacatatactacagtggtaccactagttacaacccatctctcaaaagt

SEQ ID NO: 28 - DNA, 33 bp, synthetic construct
  misc_feature 1..33: note = CUSB0125-VH-CDR3
  source 1..33: mol_type = other DNA
aactacggtagtgccttttactactttgactac

SEQ ID NO: 29 - DNA, 378 bp, synthetic construct
  misc_feature 1..378: note = CUSB0125-VL
  source 1..378: mol_type = other DNA
atggtccttgctcagtttcttgcattcttgttgctttggtttccaggtgcaagatgtgacatcctgatgacccaatctccatcctccatgtctgtttctctgggagacacagtcaccatcacttgccatgcaagtcagggcattagtagtaatatagggtggttgcagcagaaaccagggaaatcatttaagggcctgatctatcgtggaaccaacttggaagatggagttccatcaaggttcagtggcagtggatctggagcagattattctctcaccatcagcagcctggaatctgaggattttgcggactattactgtgtacagtatgctcagtttcctccgacgttcggtggaggcaccaagctggaaatcaaa

SEQ ID NO: 30 - DNA, 33 bp, synthetic construct
  misc_feature 1..33: note = CUSB0125-VL-CDR1
  source 1..33: mol_type = other DNA
catgcaagtcagggcattagtagtaatataggg

SEQ ID NO: 31 - DNA, 21 bp, synthetic construct
  misc_feature 1..21: note = CUSB0125-VL-CDR2
  source 1..21: mol_type = other DNA
cgtggaaccaacttggaagat

SEQ ID NO: 32 - DNA, 27 bp, synthetic construct
  misc_feature 1..27: note = CUSB0125-VL-CDR3
  source 1..27: mol_type = other DNA
gtacagtatgctcagtttcctccgacg

SEQ ID NO: 33 - DNA, 408 bp, synthetic construct
  misc_feature 1..408: note = CUSB0126-VH
  source 1..408: mol_type = other DNA
atgaactttgggctgagcttgattttccttgtcctaattttaaaaggtgtccaatgtgaggtgaagctggtggagtctgggggagacttagtgaagcctggagggtccctgaaactctcctgtgcagcctctggattccctttcagtagctatctcatgtcttgggttcgccagactccggagaagaggctggagtgggtcgcagccattagtggtggtggtagagacacctactatccagacaatgtgaagggtcgattcaccatctccagagacaatgccaggaacatcctgtatctgcaaatgagcagtctgaggtctgaggacacggccgtgtattactgtgcaacaaggggctcctatgctatggactattgggggcaaggaacctcagtcaccgtctcctca

SEQ ID NO: 34 - DNA, 15 bp, synthetic construct
  misc_feature 1..15: note = CUSB0126-VH-CDR1
  source 1..15: mol_type = other DNA
agctatctcatgtct

SEQ ID NO: 35 - DNA, 51 bp, synthetic construct
  misc_feature 1..51: note = CUSB0126-VH-CDR2
  source 1..51: mol_type = other DNA
gccattagtggtggtggtagagacacctactatccagacaatgtgaagggt

SEQ ID NO: 36 - DNA, 24 bp, synthetic construct
  misc_feature 1..24: note = CUSB0126-VH-CDR3
  source 1..24: mol_type = other DNA
aggggctcctatgctatggactat

SEQ ID NO: 37 - DNA, 375 bp, synthetic construct
  misc_feature 1..375: note = CUSB0126-VL
  source 1..375: mol_type = other DNA
atgctcactcagctcctgggatttctgctgctctggtttgcaggtggtaaatgtgacattcagatgacccagtctcctgcctcccagtctgcatctctgggagaaagtgtcaccatcacatgcctggcaagtcagaccattggtacatggttagcatggtatcagcagaaaccagggaaatctcctcagatcctgatttatgctgcaaccaacttggcagatggggtcccatcaaggttcagtggtagtggatctggcacaaaattttctttcaagatcagcagcctacaggctgaagattttgtaagttattactgtcaacaactttacagtattccgtggacgttcggtggaggcaccaagctggaaatcaaa

SEQ ID NO: 38 - DNA, 33 bp, synthetic construct
  misc_feature 1..33: note = CUSB0126-VL-CDR1
  source 1..33: mol_type = other DNA
ctggcaagtcagaccattggtacatggttagca

SEQ ID NO: 39 - DNA, 21 bp, synthetic construct
  misc_feature 1..21: note = CUSB0126-VL-CDR2
  source 1..21: mol_type = other DNA
gctgcaaccaacttggcagat

SEQ ID NO: 40 - DNA, 27 bp, synthetic construct
  misc_feature 1..27: note = CUSB0126-VL-CDR3
  source 1..27: mol_type = other DNA
caacaactttacagtattccgtggacg

SEQ ID NO: 41 - DNA, 408 bp, synthetic construct
  misc_feature 1..408: note = CUSB0129-VH
  source 1..408: mol_type = other DNA
atgaactttgggctgagcttgattttccttgtcctaattttaaaaggtgtccaatgtgaggtgaagctggtggagtctgggggaggcttagtgaagcctggagggtccctgaaactctcctgtgcagcctctggattccctttcagtagctatctcatgtcttgggttcgccagactccggagaagaggctggagtgggtcgcagccattagtggtggtggtagagacacctactatccagacagtgtgaagggtcgattcaccatctccagagacaatgccaggaacatcctgtacctgcaaatgagcagtctgaggtctgaggacacggccttgtattactgtgcaagaaggggctcctatgctatggactactgggggcaaggaacctcagtcaccgtctcctca

SEQ ID NO: 42 - DNA, 15 bp, synthetic construct
  misc_feature 1..15: note = CUSB0129-VH-CDR1
  source 1..15: mol_type = other DNA
agctatctcatgtct

SEQ ID NO: 43 - DNA, 51 bp, synthetic construct
  misc_feature 1..51: note = CUSB0129-VH-CDR2
  source 1..51: mol_type = other DNA
gccattagtggtggtggtagagacacctactatccagacagtgtgaagggt

SEQ ID NO: 44 - DNA, 24 bp, synthetic construct
  misc_feature 1..24: note = CUSB0129-VH-CDR3
  source 1..24: mol_type = other DNA
aggggctcctatgctatggactac

SEQ ID NO: 45 - DNA, 375 bp, synthetic construct
  misc_feature 1..375: note = CUSB0129-VL
  source 1..375: mol_type = other DNA
atgctcactcagctcctgggattactgctgctctggtttgcaggtggtaaatgtgacattcagatgacccagtctcctgcctcccagtctgcatctctgggagaaagtgtcaccatcacatgcctggcaagtcagaccattggtacatggttagcatggtatcagcagaaaccagggaaatctcctcagatcctgatttatgatgcaaccaacttggcagatggggtcccatcaaggttcagtggtagtggatctggcacaaaattttctttcaagatcagcagcctacaggctgaagattttgttagttattactgtcaacaactttacagtattccgtggacgttcggtggaggcaccaagctggaaatcaaa

SEQ ID NO: 46 - DNA, 33 bp, synthetic construct; identical to SEQ ID NO: 38
  misc_feature 1..33: note = CUSB0129-VL-CDR1
  source 1..33: mol_type = other DNA

SEQ ID NO: 47 - DNA, 21 bp, synthetic construct
  misc_feature 1..21: note = CUSB0129-VL-CDR2
  source 1..21: mol_type = other DNA
gatgcaaccaacttggcagat

SEQ ID NO: 48 - DNA, 27 bp, synthetic construct
  misc_feature 1..27: note = CUSB0129-VL-CDR3
  source 1..27: mol_type = other DNA
caacaactttacagtattccgtggacg

SEQ ID NO: 49 - DNA, 393 bp, synthetic construct
  misc_feature 1..393: note = CUSB0136-VH
  source 1..393: mol_type = other DNA
atggctgtcctggcactactcctctgcctggtggctttcccaagctgtaccctgtcccaggtgcaactgaaggagtcaggacctggcctggtggcgccctcacagagcctgtccattacctgcactgtctctgggttctcattaaccacctatgatataatctggattcgccagtcaccaggaaagggtctggagtggcttggagtaatatggactggtggagacacaaattataattcagctttcatgtccagactgagcatcagcaaggacaactccaagagccaaattttcttaaaaatgaacagtctgcaaactgatgacacagccatatattactgtgtaagagattgggcttactggggccaagggactctggtcactgtctctaca

SEQ ID NO: 50 - DNA, 15 bp, synthetic construct
  misc_feature 1..15: note = CUSB0136-VH-CDR1
  source 1..15: mol_type = other DNA
acctatgatataatc

SEQ ID NO: 51 - DNA, 48 bp, synthetic construct
  misc_feature 1..48: note = CUSB0136-VH-CDR2
  source 1..48: mol_type = other DNA
gtaatatggactggtggagacacaaattataattcagctttcatgtcc

SEQ ID NO: 52 - DNA, 12 bp, synthetic construct
  misc_feature 1..12: note = CUSB0136-VH-CDR3
  source 1..12: mol_type = other DNA
gattgggcttac

SEQ ID NO: 53 - DNA, 393 bp, synthetic construct
  misc_feature 1..393: note = CUSB0136-VL
  source 1..393: mol_type = other DNA
atggagacagacacactcctgctatgggtgctgctgctctgggtcccaggttccacaggtaacattgtgctgacccaatctccagcttctttggctgtgtctctagggcagagggccaccatatcctgcagagccagtgaaagtgttgataattatggcaatagttttatacaatggtaccagcagaaaccaggacagccacccaaactcctcatctattttgcatccaacctacaatctggggtccctgccaggttcagtggcagtgggtctaggacagacttcaccctcaccattgatcctgtggaggctgatgatgctgcaacctattactgtcatcaaaataatgaggatccattcacgttcggctcggggacaaagttggaaataaaa

SEQ ID NO: 54 - DNA, 45 bp, synthetic construct
  misc_feature 1..45: note = CUSB0136-VL-CDR1
  source 1..45: mol_type = other DNA
agagccagtgaaagtgttgataattatggcaatagttttatacaa

SEQ ID NO: 55 - DNA, 21 bp, synthetic construct
  misc_feature 1..21: note = CUSB0136-VL-CDR2
  source 1..21: mol_type = other DNA
tttgcatccaacctacaatct

SEQ ID NO: 56 - DNA, 27 bp, synthetic construct
  misc_feature 1..27: note = CUSB0136-VL-CDR3
  source 1..27: mol_type = other DNA
catcaaaataatgaggatccattcacg

SEQ ID NO: 57 - DNA, 360 bp, synthetic construct
  misc_feature 1..360: note = CUSB0125-BMVH.1
  source 1..360: mol_type = other DNA
caagtgcagctgcaggagagcggccctggactggtcaagccttctcagaccctgtctctcacctgtaccgtgtccggctacagcatcaccagcgactacgcctggaattggatcagacagcctccaggcaagggcctggaatggatcggctatatctactattctggaacaacatcctacaaccccagcctgaaaagcagagtgacaatcagcgtggacacctccaagaaccagttcagcctgaagctgagcagcgtgaccgccgctgatacagccgtgtactactgcgccagaaactacggcagcgccttttactacttcgactactggggccagggcacaaccctgaccgtgtctagc

SEQ ID NO: 58 - DNA, 360 bp, synthetic construct
  misc_feature 1..360: note = CUSB0125-BMVH.2
  source 1..360: mol_type = other DNA
caagtgcagctgcaggagagcggccctggactggtcaagccttctcagaccctgtctctcacctgtaccgtgtccggctacagcatcaccagcgactacgcctggaattggatcagacagcctccaggcaagggcctggaatggatgggctatatctactattctggaacaacatcctacaaccccagcctgaaaagcagaatcacaatcagccgggacacctccaagaaccagttcagcctgaagctgagcagcgtgaccgccgctgatacagccacctactactgcgccagaaactacggcagcgccttttactacttcgactactggggccagggcacaaccctgaccgtgtctagc

SEQ ID NO: 59 - DNA, 360 bp, synthetic construct
  misc_feature 1..360: note = CUSB0125-BMVH.3
  source 1..360: mol_type = other DNA
caagtgcagctgcaggagagcggccctggactggtcaagccttctcagaccctgtctctcacctgtaccgtgtccggctacagcatcaccagcgactacgcctggaattggatcagacagcctccaggcaagggcctggaatggatgggctatatctactattctggaacaacatcctacaaccccagcctgaaaagcagaatctccatcacccgggacacctccaagaaccagttcagcctgaagctgagcagcgtgaccgccgctgatacagccgtgtactactgcgccagaaactacggcagcgccttttactacttcgactactggggccagggcacaaccctgaccgtgtctagc

SEQ ID NO: 60 - DNA, 360 bp, synthetic construct
  misc_feature 1..360: note = CUSB0125-BMVH.4
  source 1..360: mol_type = other DNA
caagtgcagctgcaggagagcggacctggcctggtcaagcctagccagaccctgagcctgacctgtaccgtgtctggatatagcatcacatctgactacgcctggaattggatcagacagtttccaggcaagaagctggaatggatgggctacatctactacagcggcaccacctcctacaaccccagcctgaaaagcagaatcagcatcacccgggacaccagcaagaaccagttcagcctcaagctgtcttctgtgaccgccgctgatacagccgtgtactactgcgccagaaactacggcagcgccttctactatttcgactactggggccagggcacaacactgacagtgtcctcc

SEQ ID NO: 61 - DNA, 360 bp, synthetic construct
  misc_feature 1..360: note = CUSB0125-BMVH.5
  source 1..360: mol_type = other DNA
caagtgcagctgcaggagagcggacctggcctggtcaagcctagccagaccctgagcctgacctgtaccgtgtctggatatagcatcacatctgactacgcctggaattggatcagacagtttccaggcaagaagctggaatggatgggctacatctactacagcggcaccacctcctacaaccccagcctgaaaagcagaatcagcatcacccgggacaccagcaagaaccagttcagcctccagctgtcttctgtgaccgccgctgatacagccacctactactgcgccagaaactacggcagcgccttctactatttcgactactggggccagggcacaacactgacagtgtcctcc

SEQ ID NO: 62 - DNA, 321 bp, synthetic construct
  misc_feature 1..321: note = CUSB0125-BMVL.1
  source 1..321: mol_type = other DNA
gatatccagatgacccagagccctagcagcgtgtccgccagcgtcggcgacagagtgaccattacatgtcacgcttctcagggcatcagcagcaacatcggctggtaccagcagaaacctggcaaggcccccaagctgctgatctaccggggcacaaacctggaggacggcgtgccatctagattcagcggatctggcagcggaacagacttcaccctgaccatctccagcctgcaacctgaggatttcgccacctactactgcgtgcagtatgcccagttcccccccaccttcggcggaggcaccaagctggaaatcaag

SEQ ID NO: 63 - DNA, 321 bp, synthetic construct
  misc_feature 1..321: note = CUSB0125-BMVL.2
  source 1..321: mol_type = other DNA
gatatccagatgacccagagccctagcagcgtgtccgccagcgtcggcgacagagtgaccattacatgtcacgcttctcagggcatcagcagcaacatcggctggctgcagcagaaacctggcaaggcccccaagctgctgatctaccggggcacaaacctggaggacggcgtgccatctagattcagcggatctggcagcggaacagactacaccctgaccatctccagcctgcaacctgaggatttcgccacctactactgcgtgcagtatgcccagttcccccccaccttcggcggaggcaccaagctggaaatcaag

SEQ ID NO: 64 - DNA, 321 bp, synthetic construct
  misc_feature 1..321: note = CUSB0125-BMVL.3
  source 1..321: mol_type = other DNA
gatatccagatgacccagagccctagcagcgtgtccgccagcgtcggcgacagagtgaccattacatgtcacgcttctcagggcatcagcagcaacatcggctggctgcagcagaaacctggcaaggcctttaagggcctgatctaccggggcacaaacctggaggacggcgtgccatctagattcagcggatctggcagcggaacagactacaccctgaccatctccagcctgcaacctgaggatttcgccacctactactgcgtgcagtatgcccagttcccccccaccttcggcggaggcaccaagctggaaatcaag

SEQ ID NO: 65 - DNA, 336 bp, synthetic construct
  misc_feature 1..336: note = CUSB0136-BMVH.1
  source 1..336: mol_type = other DNA
caagtgcagctgcaggagagcggacctggactggtcaagcccagcgagacactgtctctgacatgtaccgtgtctggcttcagcctgacaacctacgacatcatctggatccggcagccccctggcaagggcctggaatggatcggcgtgatctggaccggcggcgacaccaattacaacagcgccttcatgtctagagtgaccatcagcgtggacaccagcaaaaaccagttctccctcaagctgagcagcgtgaccgccgctgatacagccgtgtactactgcgccagagattgggcctattggggccagggcaccctggtgaccgtgtcctcc

SEQ ID NO: 66 - DNA, 336 bp, synthetic construct
  misc_feature 1..336: note = CUSB0136-BMVH.2
  source 1..336: mol_type = other DNA
caagtgcagctgcaggagagcggacctggactggtcaagcccagcgagacactgtctattacatgtaccgtgtctggcttcagcctgacaacctacgacatcatctggatccggcagagccctggcaagggcctggaatggctgggcgtgatctggaccggcggcgacaccaattacaacagcgccttcatgtctagactgagcatcagcaaggacaccagcaaaaaccagatctccctcaagatgagcagcctgaccgccgctgatacagccgtgtactactgcgccagagattgggcctattggggccagggcaccctggtgaccgtgtcctcc

SEQ ID NO: 67 - DNA, 336 bp, synthetic construct
  misc_feature 1..336: note = CUSB0136-BMVH.3
  source 1..336: mol_type = other DNA
caagtgcagctgcaggagagcggacctggactggtcaagcccagcgagacactgtctctgacatgtaccgtgtctggcttcagcctgacaacctacgacatcatctggatccggcagagccctggcaagggcctggaatggctgggcgtgatctggaccggcggcgacaccaattacaacagcgccttcatgtctagactgaccatcagcaaggacaccagcaaaaaccagatctccctcaagatgagcagcctgaccgccgctgatacagccgtgtactactgcgccagagattgggcctattggggccagggcaccctggtgaccgtgtcctcc

SEQ ID NO: 68 - DNA, 336 bp, synthetic construct
  misc_feature 1..336: note = CUSB0136-BMVH.4
  source 1..336: mol_type = other DNA
caagtgcagctgcaggagagcggacctggactggtcaagcccagcgagacactgtctctgacatgtaccgtgtctggcttcagcctgacaacctacgacatcatctggatccggcagagccctggcaagggcctggaatggctgggcgtgatctggaccggcggcgacaccaattacaacagcgccttcatgtctagactgagcatcagcaaggacaccagcaaaaaccagttctccctcaagatgagcagcctgaccgccgctgatacagccgtgtactactgcgccagagattgggcctattggggccagggcaccctggtgaccgtgtcctcc

SEQ ID NO: 69 - DNA, 336 bp, synthetic construct
  misc_feature 1..336: note = CUSB0136-BMVH.5
  source 1..336: mol_type = other DNA
caagtgcagctgcaggagagcggacctggactggtcaagcccagcgagacactgtctctgacatgtaccgtgtctggcttcagcctgacaacctacgacatcatctggatccggcagagccctggcaagggcctggaatggctgggcgtgatctggaccggcggcgacaccaattacaacagcgccttcatgtctagactgaccatcagcaaggacaccagcaaaaaccagttctccctcaagatgagcagcctgaccgccgctgatacagccgtgtactactgcgccagagattgggcctattggggccagggcaccctggtgaccgtgtcctcc

SEQ ID NO: 70 - DNA, 336 bp, synthetic construct
  misc_feature 1..336: note = CUSB0136-BMVH.6
  source 1..336: mol_type = other DNA
caagtgcagctgcaggagagcggacctggactggtcaagcccagcgagacactgtctctgacatgtaccgtgtctggcttcagcctgacaacctacgacatcatctggatccggcagagccctggcaagggcctggaatggctgggcgtgatctggaccggcggcgacaccaattacaacagcgccttcatgtctagactgaccatcagcaaggacaccagcaaaaaccagttctccctcaagatgagcagcgtgaccgccgctgatacagccgtgtactactgcgccagagattgggcctattggggccagggcaccctggtgaccgtgtcctcc

SEQ ID NO: 71 - DNA, 333 bp, synthetic construct
  misc_feature 1..333: note = CUSB0136-BMVL.1
  source 1..333: mol_type = other DNA
gacatcgtgatgacacagagccccgacagcctggccgtgtccctgggcgagcgggccacaattaactgtagagcttctgaaagcgtcgacaactacggcaatagcttcatccagtggtatcagcagaaacctggccagcctccaaagctgctgatctacttcgcctccaacctgcaaagcggcgtgcctgatagattcagcggctctggcagcggaacagacttcaccctgaccatcagcagcctgcaggccgaggatgtggccgtgtactactgccaccagaacaacgaggacccctttaccttcggcggcggaaccaagctcgaaatcaag

SEQ ID NO: 72 - DNA, 333 bp, synthetic construct
  misc_feature 1..333: note = CUSB0136-BMVL.2
  source 1..333: mol_type = other DNA
gacatcgtgctgacacagagccccgacagcctggccgtgtccctgggcgagcgggccacaatttcttgtagagcttctgaaagcgtcgacaactacggcaatagcttcatccagtggtatcagcagaaacctggccagcctccaaagctgctgatctacttcgcctccaacctgcaaagcggcgtgcctgatagattcagcggctctggcagcggaacagacttcaccctgaccatcagcagcgtgcaggccgaggatgtggccacctactactgccaccagaacaacgaggacccctttaccttcggcggcggaaccaagctcgaaatcaag

SEQ ID NO: 73 - DNA, 333 bp, synthetic construct
  misc_feature 1..333: note = CUSB0136-BMVL.3
  source 1..333: mol_type = other DNA
gacatcgtgctgacacagagccccgacagcctggccgtgtccctgggcgagcgggccacaatttcttgtagagcttctgaaagcgtcgacaactacggcaatagcttcatccagtggtatcagcagaaacctggccagcctccaaagctgctgatctacttcgcctccaacctgcaaagcggcgtgcctgatagattcagcggctctggcagcggaacagacttcaccctgaccatcagcagcctgcaggccgaggatgtggccacctactactgccaccagaacaacgaggacccctttaccttcggcggcggaaccaagctcgaaatcaag

SEQ ID NO: 74 - DNA, 336 bp, synthetic construct
  misc_feature 1..336: note = CUSB0136-BMVH.3a
  source 1..336: mol_type = other DNA
caagtgcagctgcaggagagcggccctggcctggtcaagccttctgaaaccctgagcctgacctgtacagtgtctggattcagcctgacaacctacgacatcatttggatccggcagagccccggaaaaggcctcgagtggctgggcgtgatctggaccggcggcgacacctggtacaacagcgccttcatgtctagactgaccatctccaaggacacaagcaagaaccagatcagcctgaagatgagcagcctgacagccgctgataccgccgtgtactactgcgccagagattgggcctattggggccagggcacactggtgaccgtgtccagc

SEQ ID NO: 75 - DNA, 336 bp, synthetic construct
  misc_feature 1..336: note = CUSB0136-BMVH.3b
  source 1..336: mol_type = other DNA
caagtgcagctgcaggagtccggacctggcctggtcaagcctagcgagacactgagcctcacctgtaccgtgtccggcttcagcctgacagtgtacgacatcatctggatccggcagagccccggaaagggcctggaatggctgggcgtgatctggaccggcggcgacacaacatacaacagcgccttcatgtctagactgaccatcagcaaggacaccagcaaaaaccagatttctctgaagatgagcagcctgacagccgctgataccgccgtgtattactgcgccagagattggatctactggggccagggcaccctggtgaccgtgtctagc

SEQ ID NO: 76 - DNA, 336 bp, synthetic construct
  misc_feature 1..336: note = CUSB0136-BMVH.3c
  source 1..336: mol_type = other DNA
caagtgcagctgcaggagagcggacctggactggtcaagcccagcgagacactgagcctgacctgtaccgtgtctggcttcagcctgacagtgtacgacatcatctggatccggcagagccctggcaagggcctggaatggctgggcgtgatctggaccggcggcgacaccacatacaacagcgccttcatgtctagactgaccatcagcaaggacacaagcaaaaaccagatttctctcaagatgagcagcctgaccgccgctgatacagccgtgtattactgcgccagagattgggcctactggggccagggcaccctggtgaccgtgtcctcc

SEQ ID NO: 77 - DNA, 336 bp, synthetic construct
  misc_feature 1..336: note = CUSB0136-BMVH.3d
  source 1..336: mol_type = other DNA
caagtgcagctgcaggagagcggccctggcctggtcaagcccagcgagacactgagcctgacctgtaccgtgtccggcttcagcctgaccgtgtacgacatcatttggatccggcagagccctggaaaaggcctggaatggctgggcgtgatctggaccggcggagatacaacatacaacagcgccttcatgtctagactgacaatctccaaggacaccagcaagaaccagatcagcctcaagatgagcagcctgacagccgctgataccgccgtgtattactgcgccagagactggatgtactggggccagggcaccctggtgaccgtgtcttct

SEQ ID NO: 78 - DNA, 333 bp, synthetic construct
  misc_feature 1..333: note = CUSB0136-BMVL.2a
  source 1..333: mol_type = other DNA
gacatcgtgctgacccagagccccgacagcctggccgtgtccctgggcgagcgggccacaatttcttgtagagcttctgaatccgtggacaactggggcaatagcttcatccagtggtatcagcagaaacctggccagcctccaaagctgctgatctacttcgccagctacctccaatctggcgtccccgatagattcagcggaagcggcagcggcaccgacttcaccctgacaatcagcagcgtgcaggccgaggacgtggccacctactactgccaccagaacaacgaggatccttttaccttcggcggaggcacaaagctggaaatcaag

SEQ ID NO: 79 - DNA, 333 bp, synthetic construct
  misc_feature 1..333: note = CUSB0136-BMVL.2b
  source 1..333: mol_type = other DNA
gacatcgtgctgacccagagccccgacagcctggccgtgtccctcggcgagcgggccacaatttcttgtagagcttccgagagcgtggacaactacggcaatagcttcatccagtggtatcagcagaaacctggccagcctccaaagctgctgatctacttcgccagctacctgcaatctggcgtccctgatagattcagcggcagcggctctggcaccgacttcaccctgacaatcagcagcgtgcaggccgaagatgtggccacctactactgccaccagaacaacgaggacccctttaccttcggcggaggaacaaagctggaaatcaag

SEQ ID NO: 80 - protein, 117 aa, synthetic construct
  REGION 1..117: note = CUSB0103-VH
DVKLVESGGGLVKPGGSLKLSCAASGFTFSSYTMSWVRQTPEKRLEWVAAISGGGSYTYYADSVKGRFTISRDNAKNTLSLQMSSLKSEDTAMYFCTSPYDFAMDYWGQGTSVTVSS

SEQ ID NO: 81 - protein, 5 aa, synthetic construct
  REGION 1..5: note = CUSB0103-VH-CDR1
SYTMS

SEQ ID NO: 82 - protein, 17 aa, synthetic construct
  REGION 1..17: note = CUSB0103-VH-CDR2
AISGGGSYTYYADSVKG

SEQ ID NO: 83 - protein, 8 aa, synthetic construct
  REGION 1..8: note = CUSB0103-VH-CDR3
PYDFAMDY

SEQ ID NO: 84 - protein, 107 aa, synthetic construct
  REGION 1..107: note = CUSB0103-VL
DIQMTQTTSSLSASLGDRVTISCRASQDITNYLNWYQQKPDGSVKLLIYYTSRLHSGVPSRFRGSGSGTDYSLTISNLEQEDIATYFCQQGNTLPWTFGGGTKLEIK

SEQ ID NO: 85 - protein, 11 aa, synthetic construct
  REGION 1..11: note = CUSB0103-VL-CDR1
RASQDITNYLN

SEQ ID NO: 86 - protein, 7 aa, synthetic construct
  REGION 1..7: note = CUSB0103-VL-CDR2
YTSRLHS

SEQ ID NO: 87 - protein, 9 aa, synthetic construct
  REGION 1..9: note = CUSB0103-VL-CDR3
QQGNTLPWT

SEQ ID NO: 88 - protein, 120 aa, synthetic construct
  REGION 1..120: note = CUSB0123-VH
QVQLQQSGPELVRPGESVKISCKGSGYTFTDYVMHWVKQSHAKSLEWIGVITIYNDITNYNQKFKGKATMTIDKSSSTAYMELARLTSEDSAIYYCAREGYDSYWYFDVWGAGTTVTVSS

SEQ ID NO: 89 - protein, 5 aa, synthetic construct
  REGION 1..5: note = CUSB0123-VH-CDR1
DYVMH

SEQ ID NO: 90 - protein, 17 aa, synthetic construct
  REGION 1..17: note = CUSB0123-VH-CDR2
VITIYNDITNYNQKFKG

SEQ ID NO: 91 - protein, 11 aa, synthetic construct
  REGION 1..11: note = CUSB0123-VH-CDR3
EGYDSYWYFDV

SEQ ID NO: 92 - protein, 112 aa, synthetic construct
  REGION 1..112: note = CUSB0123-VL
DVLMTQTPLSLPVSLGDQASISCRSSQTIVHSNGNTYLEWYLQKPGQSPKLLIYKVSNRFSGVPDRFSGSGSGTDFTLKISRVEAEDLGVYYCFQGSHLPYTFGGGTKLEIK

SEQ ID NO: 93 - protein, 16 aa, synthetic construct
  REGION 1..16: note = CUSB0123-VL-CDR1
RSSQTIVHSNGNTYLE

SEQ ID NO: 94 - protein, 7 aa, synthetic construct
  REGION 1..7: note = CUSB0123-VL-CDR2
KVSNRFS

SEQ ID NO: 95 - protein, 9 aa, synthetic construct
  REGION 1..9: note = CUSB0123-VL-CDR3
FQGSHLPYT

SEQ ID NO: 96 - protein, 117 aa, synthetic construct
  REGION 1..117: note = CUSB0124-VH
QIQLVQSGPELKKPGETVKISCKASGYIFTNYGVTWVKQTPGKGLKWMTWINTNTGEPTSAEEFKGRFDFSSETSATTAYLQINNLKNEDTATYFCVRIRENAMDYWGQGTSVTVSS

SEQ ID NO: 97 - protein, 5 aa, synthetic construct
  REGION 1..5: note = CUSB0124-VH-CDR1
NYGVT

SEQ ID NO: 98 - protein, 17 aa, synthetic construct
  REGION 1..17: note = CUSB0124-VH-CDR2
WINTNTGEPTSAEEFKG

SEQ ID NO: 99 - protein, 8 aa, synthetic construct
  REGION 1..8: note = CUSB0124-VH-CDR3
IRENAMDY

SEQ ID NO: 100 - protein, 107 aa, synthetic construct
  REGION 1..107: note = CUSB0124-VL
DIQMTQSPASQSASLGESVTITCLASQTIGTWLAWYQQKPGKSPQLLIYAATRLAVGVPSRFSGSGSGTKFSFKISRLQAEDFVSYYCQQLYSTPFTFGSGTKLEIK

SEQ ID NO: 101 - protein, 11 aa, synthetic construct
  REGION 1..11: note = CUSB0124-VL-CDR1
LASQTIGTWLA

SEQ ID NO: 102 - protein, 7 aa, synthetic construct
  REGION 1..7: note = CUSB0124-VL-CDR2
AATRLAV

SEQ ID NO: 103 - protein, 9 aa, synthetic construct
  REGION 1..9: note = CUSB0124-VL-CDR3
QQLYSTPFT

SEQ ID NO: 104 - protein, 120 aa, synthetic construct
  REGION 1..120: note = CUSB0125-VH
DVQLRESGPGLVKPSQSLSLTCTVTGYSITSDYAWNWIRQFPGNKLEWMGYIYYSGTTSYNPSLKSRISITRDTSKNQFFLQLNSVTTEDTATYYCARNYGSAFYYFDYWGQGTTLTVSS

SEQ ID NO: 105 - protein, 6 aa, synthetic construct
  REGION 1..6: note = CUSB0125-VH-CDR1
SDYAWN

SEQ ID NO: 106 - protein, 16 aa, synthetic construct
  REGION 1..16: note = CUSB0125-VH-CDR2
YIYYSGTTSYNPSLKS

SEQ ID NO: 107 - protein, 11 aa, synthetic construct
  REGION 1..11: note = CUSB0125-VH-CDR3
NYGSAFYYFDY

SEQ ID NO: 108 - protein, 107 aa, synthetic construct
  REGION 1..107: note = CUSB0125-VL
DILMTQSPSSMSVSLGDTVTITCHASQGISSNIGWLQQKPGKSFKGLIYRGTNLEDGVPSRFSGSGSGADYSLTISSLESEDFADYYCVQYAQFPPTFGGGTKLEIK

SEQ ID NO: 109 - protein, 11 aa, synthetic construct
  REGION 1..11: note = CUSB0125-VL-CDR1
HASQGISSNIG

SEQ ID NO: 110 - protein, 7 aa, synthetic construct
  REGION 1..7: note = CUSB0125-VL-CDR2
RGTNLED

SEQ ID NO: 111 - protein, 9 aa, synthetic construct
  REGION 1..9: note = CUSB0125-VL-CDR3
VQYAQFPPT

SEQ ID NO: 112 - protein, 116 aa, synthetic construct
  REGION 1..116: note = CUSB0126-VH
VKLVESGGDLVKPGGSLKLSCAASGFPFSSYLMSWVRQTPEKRLEWVAAISGGGRDTYYPDNVKGRFTISRDNARNILYLQMSSLRSEDTAVYYCATRGSYAMDYWGQGTSVTVSS

SEQ ID NO: 113 - protein, 5 aa, synthetic construct
  REGION 1..5: note = CUSB0126-VH-CDR1
SYLMS

SEQ ID NO: 114 - protein, 17 aa, synthetic construct
  REGION 1..17: note = CUSB0126-VH-CDR2
AISGGGRDTYYPDNVKG

SEQ ID NO: 115 - protein, 8 aa, synthetic construct
  REGION 1..8: note = CUSB0126-VH-CDR3
RGSYAMDY

SEQ ID NO: 116 - protein, 107 aa, synthetic construct
  REGION 1..107: note = CUSB0126-VL
DIQMTQSPASQSASLGESVTITCLASQTIGTWLAWYQQKPGKSPQILIYAATNLADGVPSRFSGSGSGTKFSFKISSLQAEDFVSYYCQQLYSIPWTFGGGTKLEIK

SEQ ID NO: 117 - protein, 11 aa, synthetic construct
  REGION 1..11: note = CUSB0126-VL-CDR1
LASQTIGTWLA

SEQ ID NO: 118 - protein, 7 aa, synthetic construct
  REGION 1..7: note = CUSB0126-VL-CDR2
AATNLAD

SEQ ID NO: 119 - protein, 9 aa, synthetic construct
  REGION 1..9: note = CUSB0126-VL-CDR3
QQLYSIPWT

SEQ ID NO: 120 - protein, 116 aa, synthetic construct
  REGION 1..116: note = CUSB0129-VH
VKLVESGGGLVKPGGSLKLSCAASGFPFSSYLMSWVRQTPEKRLEWVAAISGGGRDTYYPDSVKGRFTISRDNARNILYLQMSSLRSEDTALYYCARRGSYAMDYWGQGTSVTVSS

SEQ ID NO: 121 - protein, 5 aa, synthetic construct
  REGION 1..5: note = CUSB0129-VH-CDR1
SYLMS

SEQ ID NO: 122 - protein, 17 aa, synthetic construct
  REGION 1..17: note = CUSB0129-VH-CDR2
AISGGGRDTYYPDSVKG

SEQ ID NO: 123 - protein, 8 aa, synthetic construct
  REGION 1..8: note = CUSB0129-VH-CDR3
RGSYAMDY

SEQ ID NO: 124 - protein, 107 aa, synthetic construct
  REGION 1..107: note = CUSB0129-VL
DIQMTQSPASQSASLGESVTITCLASQTIGTWLAWYQQKPGKSPQILIYDATNLADGVPSRFSGSGSGTKFSFKISSLQAEDFVSYYCQQLYSIPWTFGGGTKLEIK

SEQ ID NO: 125 - protein, 11 aa, synthetic construct
  REGION 1..11: note = CUSB0129-VL-CDR1
LASQTIGTWLA

SEQ ID NO: 126 - protein, 7 aa, synthetic construct
  REGION 1..7: note = CUSB0129-VL-CDR2
DATNLAD

SEQ ID NO: 127 - protein, 9 aa, synthetic construct
  REGION 1..9: note = CUSB0129-VL-CDR3
QQLYSIPWT

SEQ ID NO: 128 - protein, 112 aa, synthetic construct
  REGION 1..112: note = CUSB0136-VH
QVQLKESGPGLVAPSQSLSITCTVSGFSLTTYDIIWIRQSPGKGLEWLGVIWTGGDTNYNSAFMSRLSISKDNSKSQIFLKMNSLQTDDTAIYYCVRDWAYWGQGTLVTVST

SEQ ID NO: 129 - protein, 5 aa, synthetic construct
  REGION 1..5: note = CUSB0136-VH-CDR1
TYDII

SEQ ID NO: 130 - protein, 16 aa, synthetic construct
  REGION 1..16: note = CUSB0136-VH-CDR2
VIWTGGDTNYNSAFMS

SEQ ID NO: 131 - protein, 4 aa, synthetic construct
  REGION 1..4: note = CUSB0136-VH-CDR3
DWAY

SEQ ID NO: 132 - protein, 111 aa, synthetic construct
  REGION 1..111: note = CUSB0136-VL
NIVLTQSPASLAVSLGQRATISCRASESVDNYGNSFIQWYQQKPGQPPKLLIYFASNLQSGVPARFSGSGSRTDFTLTIDPVEADDAATYYCHQNNEDPFTFGSGTKLEIK

SEQ ID NO: 133 - protein, 15 aa, synthetic construct
  REGION 1..15: note = CUSB0136-VL-CDR1
RASESVDNYGNSFIQ

SEQ ID NO: 134 - protein, 7 aa, synthetic construct
  REGION 1..7: note = CUSB0136-VL-CDR2
FASNLQS

SEQ ID NO: 135 - protein, 9 aa, synthetic construct
  REGION 1..9: note = CUSB0136-VL-CDR3
HQNNEDPFT

SEQ ID NO: 136 - protein, 120 aa, synthetic construct
  REGION 1..120: note = CUSB0125-BMVH.1
QVQLQESGPGLVKPSQTLSLTCTVSGYSITSDYAWNWIRQPPGKGLEWIGYIYYSGTTSYNPSLKSRVTISVDTSKNQFSLKLSSVTAADTAVYYCARNYGSAFYYFDYWGQGTTLTVSS

SEQ ID NO: 137 - protein, 120 aa, synthetic construct
  REGION 1..120: note = CUSB0125-BMVH.2
QVQLQESGPGLVKPSQTLSLTCTVSGYSITSDYAWNWIRQPPGKGLEWMGYIYYSGTTSYNPSLKSRITISRDTSKNQFSLKLSSVTAADTATYYCARNYGSAFYYFDYWGQGTTLTVSS

SEQ ID NO: 138 - protein, 120 aa, synthetic construct
  REGION 1..120: note = CUSB0125-BMVH.3
QVQLQESGPGLVKPSQTLSLTCTVSGYSITSDYAWNWIRQPPGKGLEWMGYIYYSGTTSYNPSLKSRISITRDTSKNQFSLKLSSVTAADTAVYYCARNYGSAFYYFDYWGQGTTLTVSS

SEQ ID NO: 139 - protein, 120 aa, synthetic construct
  REGION 1..120: note = CUSB0125-BMVH.4
QVQLQESGPGLVKPSQTLSLTCTVSGYSITSDYAWNWIRQFPGKKLEWMGYIYYSGTTSYNPSLKSRISITRDTSKNQFSLKLSSVTAADTAVYYCARNYGSAFYYFDYWGQGTTLTVSS

SEQ ID NO: 140 - protein, 120 aa, synthetic construct
  REGION 1..120: note = CUSB0125-BMVH.5
QVQLQESGPGLVKPSQTLSLTCTVSGYSITSDYAWNWIRQFPGKKLEWMGYIYYSGTTSYNPSLKSRISITRDTSKNQFSLQLSSVTAADTATYYCARNYGSAFYYFDYWGQGTTLTVSS

SEQ ID NO: 141 - protein, 107 aa, synthetic construct
  REGION 1..107: note = CUSB0125-BMVL.1
DIQMTQSPSSVSASVGDRVTITCHASQGISSNIGWYQQKPGKAPKLLIYRGTNLEDGVPSRFSGSGSGTDFTLTISSLQPEDFATYYCVQYAQFPPTFGGGTKLEIK

SEQ ID NO: 142 - protein, 107 aa, synthetic construct
  REGION 1..107: note = CUSB0125-BMVL.2
DIQMTQSPSSVSASVGDRVTITCHASQGISSNIGWLQQKPGKAPKLLIYRGTNLEDGVPSRFSGSGSGTDYTLTISSLQPEDFATYYCVQYAQFPPTFGGGTKLEIK

SEQ ID NO: 143 - protein, 107 aa, synthetic construct
  REGION 1..107: note = CUSB0125-BMVL.3
DIQMTQSPSSVSASVGDRVTITCHASQGISSNIGWLQQKPGKAFKGLIYRGTNLEDGVPSRFSGSGSGTDYTLTISSLQPEDFATYYCVQYAQFPPTFGGGTKLEIK

SEQ ID NO: 144 - protein, 112 aa, synthetic construct
  REGION 1..112: note = CUSB0136-BMVH.1
QVQLQESGPGLVKPSETLSLTCTVSGFSLTTYDIIWIRQPPGKGLEWIGVIWTGGDTNYNSAFMSRVTISVDTSKNQFSLKLSSVTAADTAVYYCARDWAYWGQGTLVTVSS

SEQ ID NO: 145 - protein, 112 aa, synthetic construct
  REGION 1..112: note = CUSB0136-BMVH.2
QVQLQESGPGLVKPSETLSITCTVSGFSLTTYDIIWIRQSPGKGLEWLGVIWTGGDTNYNSAFMSRLSISKDTSKNQISLKMSSLTAADTAVYYCARDWAYWGQGTLVTVSS

SEQ ID NO: 146 - protein, 112 aa, synthetic construct
  REGION 1..112: note = CUSB0136-BMVH.3
QVQLQESGPGLVKPSETLSLTCTVSGFSLTTYDIIWIRQSPGKGLEWLGVIWTGGDTNYNSAFMSRLTISKDTSKNQISLKMSSLTAADTAVYYCARDWAYWGQGTLVTVSS

SEQ ID NO: 147 - protein, 112 aa, synthetic construct
  REGION 1..112: note = CUSB0136-BMVH.4
QVQLQESGPGLVKPSETLSLTCTVSGFSLTTYDIIWIRQSPGKGLEWLGVIWTGGDTNYNSAFMSRLSISKDTSKNQFSLKMSSLTAADTAVYYCARDWAYWGQGTLVTVSS

SEQ ID NO: 148 - protein, 112 aa, synthetic construct
  REGION 1..112: note = CUSB0136-BMVH.5
QVQLQESGPGLVKPSETLSLTCTVSGFSLTTYDIIWIRQSPGKGLEWLGVIWTGGDTNYNSAFMSRLTISKDTSKNQFSLKMSSLTAADTAVYYCARDWAYWGQGTLVTVSS

SEQ ID NO: 149 - protein, 112 aa, synthetic construct
  REGION 1..112: note = CUSB0136-BMVH.6
QVQLQESGPGLVKPSETLSLTCTVSGFSLTTYDIIWIRQSPGKGLEWLGVIWTGGDTNYNSAFMSRLTISKDTSKNQFSLKMSSVTAADTAVYYCARDWAYWGQGTLVTVSS

SEQ ID NO: 150 - protein, 111 aa, synthetic construct
  REGION 1..111: note = CUSB0136-BMVL.1
DIVMTQSPDSLAVSLGERATINCRASESVDNYGNSFIQWYQQKPGQPPKLLIYFASNLQSGVPDRFSGSGSGTDFTLTISSLQAEDVAVYYCHQNNEDPFTFGGGTKLEIK

SEQ ID NO: 151 - protein, 111 aa, synthetic construct
  REGION 1..111: note = CUSB0136-BMVL.2
DIVLTQSPDSLAVSLGERATISCRASESVDNYGNSFIQWYQQKPGQPPKLLIYFASNLQSGVPDRFSGSGSGTDFTLTISSVQAEDVATYYCHQNNEDPFTFGGGTKLEIK

SEQ ID NO: 152 - protein, 111 aa, synthetic construct
  REGION 1..111: note = CUSB0136-BMVL.3
DIVLTQSPDSLAVSLGERATISCRASESVDNYGNSFIQWYQQKPGQPPKLLIYFASNLQSGVPDRFSGSGSGTDFTLTISSLQAEDVATYYCHQNNEDPFTFGGGTKLEIK

SEQ ID NO: 153 - protein, 112 aa, synthetic construct
  REGION 1..112: note = CUSB0136-BMVH.3a
QVQLQESGPGLVKPSETLSLTCTVSGFSLTTYDIIWIRQSPGKGLEWLGVIWTGGDTWYNSAFMSRLTISKDTSKNQISLKMSSLTAADTAVYYCARDWAYWGQGTLVTVSS

SEQ ID NO: 154 - protein, 112 aa, synthetic construct
  REGION 1..112: note = CUSB0136-BMVH.3b
QVQLQESGPGLVKPSETLSLTCTVSGFSLTVYDIIWIRQSPGKGLEWLGVIWTGGDTTYNSAFMSRLTISKDTSKNQISLKMSSLTAADTAVYYCARDWIYWGQGTLVTVSS

SEQ ID NO: 155 - protein, 112 aa, synthetic construct
  REGION 1..112: note = CUSB0136-BMVH.3c
QVQLQESGPGLVKPSETLSLTCTVSGFSLTVYDIIWIRQSPGKGLEWLGVIWTGGDTTYNSAFMSRLTISKDTSKNQISLKMSSLTAADTAVYYCARDWAYWGQGTLVTVSS

SEQ ID NO: 156 - protein, 112 aa, synthetic construct
  REGION 1..112: note = CUSB0136-BMVH.3d
QVQLQESGPGLVKPSETLSLTCTVSGFSLTVYDIIWIRQSPGKGLEWLGVIWTGGDTTYNSAFMSRLTISKDTSKNQISLKMSSLTAADTAVYYCARDWMYWGQGTLVTVSS

SEQ ID NO: 157 - protein, 111 aa, synthetic construct
  REGION 1..111: note = CUSB0136-BMVL.2a
DIVLTQSPDSLAVSLGERATISCRASESVDNWGNSFIQWYQQKPGQPPKLLIYFASYLQSGVPDRFSGSGSGTDFTLTISSVQAEDVATYYCHQNNEDPFTFGGGTKLEIK

SEQ ID NO: 158 - protein, 111 aa, synthetic construct
  REGION 1..111: note = CUSB0136-BMVL.2b
DIVLTQSPDSLAVSLGERATISCRASESVDNYGNSFIQWYQQKPGQPPKLLIYFASYLQSGVPDRFSGSGSGTDFTLTISSVQAEDVATYYCHQNNEDPFTFGGGTKLEIK

SEQ ID NO: 159 - protein, 6 aa, synthetic construct
SDYAWN

SEQ ID NO: 160 - protein, 16 aa, synthetic construct
YIYYSGTTSYNPSLKS

SEQ ID NO: 161 - protein, 11 aa, synthetic construct
NYGSAFYYFDY

SEQ ID NO: 162 - protein, 6 aa, synthetic construct
SDYAWN

SEQ ID NO: 163 - protein, 16 aa, synthetic construct
YIYYSGTTSYNPSLKS

SEQ ID NO: 164 - protein, 11 aa, synthetic construct
NYGSAFYYFDY

SEQ ID NO: 165 - protein, 6 aa, synthetic construct
SDYAWN

SEQ ID NO: 166 - protein, 16 aa, synthetic construct
YIYYSGTTSYNPSLKS

SEQ ID NO: 167 - protein, 11 aa, synthetic construct
NYGSAFYYFDY

SEQ ID NO: 168 - protein, 6 aa, synthetic construct
SDYAWN

SEQ ID NO: 169 - protein, 16 aa, synthetic construct
YIYYSGTTSYNPSLKS

SEQ ID NO: 170 - protein, 11 aa, synthetic construct
NYGSAFYYFDY

SEQ ID NO: 171 - protein, 6 aa, synthetic construct
SDYAWN

SEQ ID NO: 172 - protein, 16 aa, synthetic construct
YIYYSGTTSYNPSLKS

SEQ ID NO: 173 - protein, 11 aa, synthetic construct
NYGSAFYYFDY

SEQ ID NO: 174 - protein, 11 aa, synthetic construct
HASQGISSNIG

SEQ ID NO: 175 - protein, 7 aa, synthetic construct
RGTNLED

SEQ ID NO: 176 - protein, 9 aa, synthetic construct
VQYAQFPPT

SEQ ID NO: 177 - protein, 11 aa, synthetic construct
HASQGISSNIG

SEQ ID NO: 178 - protein, 7 aa, synthetic construct
RGTNLED

SEQ ID NO: 179 - protein, 9 aa, synthetic construct
VQYAQFPPT

SEQ ID NO: 180 - protein, 11 aa, synthetic construct
HASQGISSNIG

SEQ ID NO: 181 - protein, 7 aa, synthetic construct
RGTNLED

SEQ ID NO: 182 - protein, 9 aa, synthetic construct
VQYAQFPPT

SEQ ID NO: 183 - protein, 5 aa, synthetic construct
TYDII

SEQ ID NO: 184 - protein, 16 aa, synthetic construct
VIWTGGDTNYNSAFMS

SEQ ID NO: 185 - protein, 4 aa, synthetic construct
DWAY

SEQ ID NO: 186 - protein, 5 aa, synthetic construct
TYDII

SEQ ID NO: 187 - protein, 16 aa, synthetic construct
VIWTGGDTNYNSAFMS

SEQ ID NO: 188 - protein, 4 aa, synthetic construct
DWAY

SEQ ID NO: 189 - protein, 5 aa, synthetic construct
TYDII

SEQ ID NO: 190 - protein, 16 aa, synthetic construct
VIWTGGDTNYNSAFMS

SEQ ID NO: 191 - protein, 4 aa, synthetic construct
DWAY

SEQ ID NO: 192 - protein, 5 aa, synthetic construct
TYDII

SEQ ID NO: 193 - protein, 16 aa, synthetic construct
VIWTGGDTNYNSAFMS

SEQ ID NO: 194 - protein, 4 aa, synthetic construct
DWAY

SEQ ID NO: 195 - protein, 5 aa, synthetic construct
TYDII

SEQ ID NO: 196 - protein, 16 aa, synthetic construct
VIWTGGDTNYNSAFMS

SEQ ID NO: 197 - protein, 4 aa, synthetic construct
DWAY

SEQ ID NO: 198 - protein, 5 aa, synthetic construct
TYDII

SEQ ID NO: 199 - protein, 16 aa, synthetic construct
VIWTGGDTNYNSAFMS

SEQ ID NO: 200 - protein, 4 aa, synthetic construct
DWAY

SEQ ID NO: 201 - protein, 11 aa, synthetic construct
CUSBBMVHCDR

SEQ ID NO: 202 - protein, 7 aa, synthetic construct
FASNLQS

SEQ ID NO: 203 - protein, 9 aa, synthetic construct
HQNNEDPFT

SEQ ID NO: 204 - protein, 15 aa, synthetic construct
RASESVDNYGNSFIQ

SEQ ID NO: 205 - protein, 7 aa, synthetic construct
FASNLQS

SEQ ID NO: 206 - protein, 9 aa, synthetic construct
HQNNEDPFT

SEQ ID NO: 207 - protein, 15 aa, synthetic construct
RASESVDNYGNSFIQ

SEQ ID NO: 208 - protein, 7 aa, synthetic construct
FASNLQS

SEQ ID NO: 209 - protein, 9 aa, synthetic construct
HQNNEDPFT

SEQ ID NO: 210 - protein, 5 aa, synthetic construct
TYDII

SEQ ID NO: 211 - protein, 16 aa, synthetic construct
VIWTGGDTWYNSAFMS

SEQ ID NO: 212 - protein, 4 aa, synthetic construct
DWAY

SEQ ID NO: 213 - protein, 5 aa, synthetic construct
VYDII

SEQ ID NO: 214 - protein, 16 aa, synthetic construct
VIWTGGDTTYNSAFMS

SEQ ID NO: 215 - protein, 4 aa, synthetic construct
DWIY

SEQ ID NO: 216 - protein, 5 aa, synthetic construct
VYDII

SEQ ID NO: 217 - protein, 16 aa, synthetic construct
VIWTGGDTTYNSAFMS

SEQ ID NO: 218 - protein, 4 aa, synthetic construct
DWAY

SEQ ID NO: 219 - protein, 5 aa, synthetic construct
VYDII

SEQ ID NO: 220 - protein, 16 aa, synthetic construct
VIWTGGDTTYNSAFMS

SEQ ID NO: 221 - protein, 4 aa, synthetic construct
DWMY

SEQ ID NO: 222 - protein, 15 aa, synthetic construct
RASESVDNWGNSFIQ

SEQ ID NO: 223 - protein, 7 aa, synthetic construct
FASYLQS

SEQ ID NO: 224 - protein, 9 aa, synthetic construct
HQNNEDPFT

SEQ ID NO: 225 - protein, 15 aa, synthetic construct
RASESVDNYGNSFIQ

SEQ ID NO: 226 - protein, 7 aa, synthetic construct
FASYLQS

SEQ ID NO: 227 - protein, 9 aa, synthetic construct
HQNNEDPFT

SEQ ID NO: 228 - protein, 5 aa, synthetic construct
  VARIANT 1: note = X is T or V
XYDII

SEQ ID NO: 229 - protein, 16 aa, synthetic construct
  VARIANT 9: note = X is N, W or T
VIWTGGDTXYNSAFMS

SEQ ID NO: 230 - skipped

SEQ ID NO: 231 - protein, 15 aa, synthetic construct
  VARIANT 9: note = X is Y or W
RASESVDNXGNSFIQ

SEQ ID NO: 232 - protein, 7 aa, synthetic construct
  VARIANT 4: note = X5 is N, Y, H or Q
FASXLQS

SEQ ID NO: 233 - protein, 120 aa, synthetic construct
  VARIANT 1: note = X is D or Q
  VARIANT 5: note = X is R or Q
  VARIANT 17: note = X is S or T
  VARIANT 25: note = X is T or S
  VARIANT 41: note = X is F or P
  VARIANT 44: note = X is N or K
  VARIANT 45: note = is K or G
  VARIANT 49: note = X is M or I
  VARIANT 68: note = X is I or V
  VARIANT 69: note = X is S or T
  VARIANT 71: note = X is T or S
  VARIANT 72: note = X is R or V
  VARIANT 80: note = X is F or S
  VARIANT 82: note = X is Q or K
  VARIANT 84: note = X is N or S
  VARIANT 88: note = X is T or A
  VARIANT 89: note = X is E or A
  VARIANT 93: note = X is T or V
XVQLXESGPGLVKPSQXLSLTCTVXGYSITSDYAWNWIRQXPGXXLEWXGYIYYSGTTSYNPSLKSRXXIXXDTSKNQFXLXLXSVTXXDTAXYYCARNYGSAFYYFDYWGQGTTLTVSS

SEQ ID NO: 234 - protein, 107 aa, synthetic construct
  VARIANT 11: note = X is M or V
  VARIANT 13: note = X is V or A
  VARIANT 15: note = X is L or V
  VARIANT 18: note = X is T or R
  VARIANT 35: note = X is L or Y
  VARIANT 43: note = X is S or A
  VARIANT 44: note = X is F or P
  VARIANT 46: note = X is G or L
  VARIANT 69: note = X is A or T
  VARIANT 70: note = X is Y or F
  VARIANT 71: note = X is S or T
  VARIANT 79: note = X is E or Q
  VARIANT 80: note = X is S or P
  VARIANT 85: note = X is D or T
  VARIANT 3: note = X is L or Q
DIXMTQSPSSXSXSXGDXVTITCHASQGISSNIGWXQQKPGKXXKXLIYRGTNLEDGVPSRFSGSGSGXDXXLTISSLXXEDFAXYYCVQYAQFPPTFGGGTKLEIK

SEQ ID NO: 235 - protein, 112 aa, synthetic construct
  VARIANT 5: note = X is K or Q
  VARIANT 13: note = X is A or K
  VARIANT 16: note = X is Q or E
  VARIANT 17: note = X is S or T
  VARIANT 20: note = X is I or L
  VARIANT 31: note = X is T or V
  VARIANT 40: note = X is S or P
  VARIANT 48: note = X is L or I
  VARIANT 58: note = X is N,W or T
  VARIANT 67: note = X is L or V
  VARIANT 68: note = X is T or S
  VARIANT 71: note = X is K or V
  VARIANT 73: note = X is N or T
  VARIANT 76: note = X is S or N
  VARIANT 78: note = X is I or F
  VARIANT 79: note = X is F or S
  VARIANT 82: note = X is M or L
  VARIANT 83: note = X is N or S
  VARIANT 85: note = X is L or V
  VARIANT 86: note = X is Q or T
  VARIANT 87: note = X is T or A
  VARIANT 88: note = X is D or A
  VARIANT 92: note = X is I or V
  VARIANT 96: note = X is A or V
  VARIANT 100: note = X is A, I or M
  VARIANT 112: note = X is T or S
QVQLXESGPGLVXPSXXLSXTCTVSGFSLTXYDIIWIRQXPGKGLEWXGVIWTGGDTXYNSAFMSRXXISXDXSKXQXXLKXXSXXXXDTAXYYCXRDWXYWGQGTLVTVSX

SEQ ID NO: 236 - protein, 111 aa, synthetic construct
  VARIANT 1: note = X is N or D
  VARIANT 4: note = X is L or M
  VARIANT 9: note = X is A or D
  VARIANT 17: note = X is Q or E
  VARIANT 22: note = X is S or N
  VARIANT 32: note = X is Y or W
  VARIANT 57: note = X is N or Y
  VARIANT 64: note = X is A or D
  VARIANT 72: note = X is R or G
  VARIANT 80: note = X is D or S
  VARIANT 81: note = X is P or S
  VARIANT 82: note = X is V or L
  VARIANT 83: note = X is E or Q
  VARIANT 85: note = X is D or E
  VARIANT 87: note = X is A or V
  VARIANT 89: note = X is T or V
  VARIANT 104: note = X is S or G
XIVXTQSPXSLAVSLGXRATIXCRASESVDNXGNSFIQWYQQKPGQPPKLLIYFASXLQSGVPXRFSGSGSXTDFTLTIXXXXAXDXAXYYCHQNNEDPFTFGXGTKLEIK